(19) United States
(12) Patent Application Publication
(10) Pub. No.: US 2015/0238948 A1
(43) Pub. Date: Aug. 27, 2015

(54) POLYMERIC AND SOLID-SUPPORTED CATALYSTS, AND METHODS OF DIGESTING LIGNIN-CONTAINING MATERIALS USING SUCH CATALYSTS

(71) Applicant: Midori Renewables, Inc., Cambridge, MA (US)

(72) Inventor: John M. Geremia, Watertown, MA (US)

(73) Assignee: MIDORI RENEWABLES, INC., Cambridge, MA (US)

(21) Appl. No.: 14/423,698

(22) PCT Filed: Aug. 23, 2013
(86) PCT No.: PCT/US2013/056462

§ 371 Date: Feb. 24, 2015

Related U.S. Application Data
(60) Provisional application No. 61/693,216, filed on Aug. 24, 2012.

(51) Int. Cl. B01J 31/10; C07C 39/19; C07C 37/00; B01J 31/08; C07C 43/23; C07C 41/01; C07D 317/50; C07C 43/215; C07H 15/203; C07D 311/10

(52) U.S. Cl. CPC B01J 31/10; C07H 15/203; C07C 39/19; C07C 37/004; C07D 311/10; C07C 43/23; C07C 41/01; C07D 317/50; C07C 43/215; B01J 31/08; B01J 2231/40; B01J 2531/004

(57) ABSTRACT

Provided herein are solid base catalysts useful in non-enzymatic break down of lignin in biomass. The solid base catalysts may be polymeric catalysts or solid-support base catalysts with ionic moieties. Provided are also methods for at least partially depolymerizing lignin materials into various lignin digestion products using the solid base catalysts described herein.

### CROSS REFERENCE TO RELATED APPLICATIONS

This application claims priority to U.S. Provisional Patent Application No. 61/693,216, filed Aug. 24, 2012, which is incorporated herein by reference in its entirety.

### FIELD

The present disclosure relates generally to catalysts that may be used in break down of lignin, and more specifically to solid catalysts with basic and ionic moieties that may be used to break down lignin.

### BACKGROUND

Saccharification of cellulosic materials, particularly biomass waste products of agriculture, forestry and waste treatment are of great economic and environmental relevance. As part of biomass energy utilization, attempts have been made to obtain ethanol (bioethanol) by hydrolyzing cellulose or hemicellulose, which are major constituents of plants. The hydrolysis products, which include sugars and simple carbohydrates, may then be subjected to further biological and/or chemical conversion to produce fuels or other commodity chemicals. For example, ethanol is utilized as a fuel or mixed into a fuel such as gasoline. Major constituents of plants include, for example, cellulose (a polymer glucose, which is a six-carbon sugar), hemicellulose (a branched polymer of five- and six-carbon sugars), lignin, and starch. Current methods for liberating sugars from lignocellulosic materials, however, are inefficient on a commercial scale based on yields, as well as the water and energy used.

One factor that can hinder hydrolysis of cellulose and hemicellulose in lignocellulosic biomass is the amount of lignin present in the biomass. Lignin is a complex chemical compound that is commonly found in lignocellulosic biomass. Lignin is typically covalently linked to cellulose or hemicellulose, cross-linking different polysaccharides within the biomass. Due to the complex cross-linking, lignin often hinders the ability of a catalyst (e.g., an enzyme catalyst or an acid catalyst) to access the cellulose and hemicellulose in ligncellulosic biomass to produce sugars. Conventional methods for hydrolysis of lignocellulosic biomass to produce sugars typically involve removal of some of the lignin present in the lignocellulosic biomass before hydrolysis. The catalysts used to hydrolyze lignocellulosic biomass to produce sugars also typically leave residual undigested lignin from the saccharification.

Thus, what is needed in the art is a commercially-viable method of breaking down lignin, either before hydrolysis to facilitate access to cellulosic materials or after hydrolysis to produce one or more lignin digestion products.

### BRIEF SUMMARY

The present disclosure addresses this need by providing catalysts that can be used to digest lignin in biomass. Specifically, the solid catalysts described herein can at least partially digest lignin into various lignin digestion products.

In one aspect, the catalysts provided herein are polymeric catalysts. The catalyst includes basic monomers and ionic monomers connected to form a polymeric backbone. Each basic monomer independently includes at least one Bronsted-Lowry base. Each Bronsted-Lowry base independently includes at least one nitrogen-containing cationic group, at least one phosphorous-containing cationic group, at least one sulfur-containing cationic group, or any combinations thereof. In some embodiments of the polymeric catalyst, one or more of the basic monomers are directly connected to the polymeric backbone. In other embodiments of the polymeric catalyst, one or more of the basic monomers further include a linker connecting the Bronsted-Lowry base to the polymeric backbone.

In other embodiments, the polymeric catalyst has a plurality of monomers, in which at least one monomer has a basic moiety, and at least one monomer includes an ionic moiety (e.g., a covalently-attached anionic group that can be coordinated to an exchangeable counter-ion). In some embodiments, the polymeric catalyst has a structure of Formula (I):

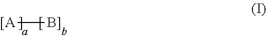

in which A represents monomer that have an basic moiety and B represents monomers that have an ionic moiety (e.g., an anionic moiety or a salt). The basic moiety includes a Bronsted-Lowry base, in which the Bronsted-Lowry base includes a nitrogen-containing functional group, a phosphorous-containing functional group, or a sulfur-containing functional group. Moreover, a and b are stochiometric coefficients, such that a and b together make up a substantial portion of the co-monomer subunits of the polymer. For example, a and b together make up at least about 30%, at least about 40%, at least about 50%, at least about 60%, at least about 70%, at least about 80%, at least about 90%, at least about 95%, at least about 99% or substantially all of the co-monomer subunits of the polymer.

In some embodiments, the polymeric catalyst of Formula (A-I) is a polymeric catalyst of Formula (A-Ia):

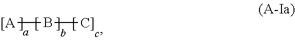

which includes monomers C that are covalently bound to and are cross-linked with other monomers in the polymeric catalyst, and c is a stoichiometric coefficient.

In other embodiments, the catalyst of formula (A-I) is a polymeric catalyst of Formula (A-Ib):

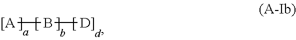

which includes monomers D that are covalently bound to other monomers in the base catalyst, and d is a stoichiometric coefficient.

In other embodiments, the catalyst of formula (A-I) is a polymeric catalyst of Formula (A-Ic):

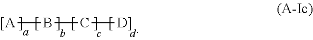

In certain embodiments, monomers D are non-functionalized moieties, such as hydrophobic moieties (e.g., phenyl).

In yet other embodiments, the catalyst has a structure of Formula (A-II):

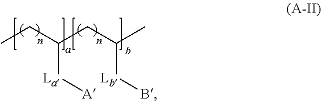

in which each of L_{a′} and L_{b′} is independently for each occurrence a linker or absent; each A′ for each occurrence is a basic moiety; each B′ for each occurrence is an ionic (e.g., anionic) moiety; each n is independently for each occurrence 0, 1, 2, 3, 4, 5, or 6; and a and b are stochiometric coefficients together make up a substantial portion of the co-monomer subunits of the polymeric catalyst. For example, a and b together make up at least about 30%, at least about 40%, at least about 50%, at least about 60%, at least about 70%, at least about 80%, at least about 90%, at least about 95%, at least about 99% or substantially all of the monomers of the polymeric catalyst. Each of L_{a′} and L_{b′} can independently have a plurality of A′ moieties and B′ moieties, respectively.

In yet other embodiments, the polymeric catalyst has a structure of Formula (A-III):

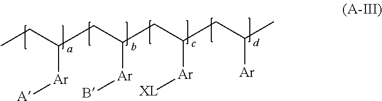

in which each Ar is independently for each occurrence an aryl or heteroaryl moiety; each A′ for each occurrence is a basic moiety; each B′ for each occurrence is an ionic moiety (e.g., an anionic moiety); each XL for each occurrence is a cross-linking moiety; and a, b, c, and d are stoichiometric coefficients, such that when taken together make up a substantial portion of the co-monomer subunits of the polymeric catalyst. For example, a, b, c, and d together make up at least about 30%, at least about 40%, at least about 50%, at least about 60%, at least about 70%, at least about 80%, at least about 90%, at least about 95%, at least about 99% or substantially all of the co-monomer subunits of the polymeric catalyst. Each Ar can independently have a plurality of A′ moieties, B′ moieties, and XL moieties, respectively.

In yet other embodiments, the polymeric catalyst has a structure of Formula (A-IV):

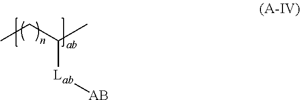

in which each of L_{ab} is independently for each occurrence a linker or absent; each AB for each occurrence is a moiety that includes an basic and an ionic moiety (e.g., an anionic moiety); each n is independently for each occurrence 0, 1, 2, 3, 4, 5, or 6; and ab is a stochiometric coefficient, such that ab makes up a substantial portion of the co-monomer subunits of the polymeric catalyst. For example, ab makes up at least about 30%, at least about 40%, at least about 50%, at least about 60%, at least about 70%, at least about 80%, at least about 90%, at least about 95%, at least about 99% or substantially all of the co-monomer subunits of the polymeric catalyst. Each of L_{ab} can independently have a plurality of basic moieties and ionic moieties (e.g., anionic moieties), respectively.

Where the polymeric catalysts such as Formula (A-I), (A-Ia), (A-Ib), (A-Ic), (A-II), (A-III), or (A-IV) are depicted herein, the connectivity as shown above does not require a block polymer, but can also include other configurations of the A and B monomers, including random polymers. Moreover, the depiction of attachment of the monomers, such as that of A to B, does not limit the nature of the attachment of the monomers, such as A to B by way of a carbon-carbon bond, but can also include other attachments such as a carbon-heteroatom bond.

In certain embodiments, the polymeric catalyst of Formula (A-I), (A-Ia), (A-Ib), (A-Ic), (A-II), (A-III), or (A-IV) can catalyze the break-down of lignin. In general, it is the basic moiety on the polymeric catalyst of Formula (A-I), (A-Ia), (A-Ib), (A-Ic), (A-II), (A-III), or (A-IV) that catalyzes the cleavage of the aryl ether linkages in lignin. However, the polymeric catalyst of Formula (A-I), (A-Ia), (A-Ib), (A-Ic), (A-II), (A-III), or (A-IV) also includes an ionic moiety (e.g., an anionic moiety), which is generally present as a sulfonate salt, a phosphonate salt, an acetate salt, an isophthalate salt, and a boronate salt. This salt functionality of the polymeric catalyst of Formula (A-I), (A-Ia), (A-Ib), (A-Ic), (A-II), (A-III), or (A-IV) can promote the break-down of the complex forming between lignin, cellulose, and hemicellulose. For example, the ionic moiety can disrupt hydrogen bonding in cellulose and hemicellulose, which can allow the basic moiety of the polymeric catalyst to access more readily the ether linkages of the lignin. Accordingly, the combination of the two functional moieties on a single polymer can provide for a catalyst that is effective in the break-down of lignin using relatively mild conditions as compared to those methods that employ a more caustic base, or methods that employ harsh conditions such as high temperatures or pressure.

In certain embodiments, the polymeric catalyst is in the form of a solid particle that includes a solid core and any of the polymeric catalysts described herein, in which the polymeric catalyst is coated on the surface of the solid core. In some embodiments of the polymeric catalyst, the solid core is made up of an inert material or a magnetic material. In one embodiment of the polymeric catalyst, the solid core is made up of iron. In some embodiments of the polymeric catalyst, the solid particle is substantially free of pores. In other embodiments the polymeric catalyst, the solid particle has catalytic activity. In certain embodiments, at least about 50%, at least 60%, at least 70%, at least 80%, at least 90% of the catalytic activity of the solid particle is present on or near the exterior surface of the solid particle.

In another aspect, the catalysts provided herein are solid-supported catalysts. The solid-supported catalyst includes a solid support, basic moieties attached to the solid support, and ionic moieties attached to the solid support. The solid support includes a material, wherein the material is selected from carbon, silica, silica gel, alumina, magnesia, titania, zirconia, clays, magnesium silicate, silicon carbide, zeolites, ceramics, and any combinations thereof. Each basic moiety independently includes at least one Bronsted-Lowry base, wherein each Bronsted-Lowry base independently includes at least one nitrogen-containing cationic group, at least one phosphorous-containing cationic group, at least one sulfur-containing cationic group, or any combinations thereof.

Provided is also a composition that includes lignin and any of the catalysts described herein. In some embodiments, the composition further includes one or more solvents. In certain embodiments, the solvent is an aqueous solvent.

Provided is also a partially-depolymerized lignin composition that includes any of the catalysts described herein, one or more lignin digestion products, and residual lignin. In some embodiments, the one or more lignin digestion products are selected from monolignols, phenylpropenes, monolignolglucosides, and any combinations thereof. In certain embodiments, the one or more lignin digestion products include p-coumaryl alcohol, coumarilin, coniferyl alchol, coniferin, sinapyl alcohol, sinaplin, eugenol, chavicol, safrole, estragol, and any combinations thereof.

In another aspect, provided is a method for at least partially depolymerizing a lignin composition, by:

a) providing a lignin composition;

b) contacting the lignin composition with any one of the catalysts described herein to form a reaction mixture;

c) degrading the lignin composition in the reaction mixture to produce a liquid phase and a solid phase, wherein the liquid phase includes one or more lignin digestion products, and the solid phase includes residual lignin;

d) isolating at least a portion of the liquid phase from the solid phase; and

e) recovering the one or more lignin digestion products from the isolated liquid phase.

In some embodiments, step (b) further includes contacting the lignin composition and the catalysts with one or more solvents to form the reaction mixture. In some embodiments, step (b) further includes contacting the lignin composition and the catalysts with water to form the reaction mixture. In some embodiments, the one or more lignin digestion products are selected from monolignols, phenylpropenes, monolignolglucosides, and any combinations thereof. In certain embodiments, the one or more lignin digestion products include p-coumaryl alcohol, coumarilin, coniferyl alchol, coniferin, sinapyl alcohol, sinaplin, eugenol, chavicol, safrole, estragol, and any combinations thereof.

Provided is also a use of any of the catalysts described herein for degrading a lignin composition into one or more lignin digestion products.

In yet another aspect, provided is a method of producing any one of the polymeric catalysts described herein, by:

a) providing a starting polymer;

b) reacting the starting polymer with a nitrogen-containing compound, a phosphorous-containing compound, a sulfur-containing compound to produce an ionic intermediate;

c) reacting the ionic intermediate with an acid to produce an acidic-ionic intermediate; and

d) washing the acidic-ionic intermediate with a base to produce any one of the polymeric catalysts described herein.

In yet another aspect, provided is a method for producing any one of the solid-supported catalysts described herein, by:

a) providing a carbonaceous material;

b) carbonizing at least a portion of the carbonaceous material to form a solid support;

b) activating at least a portion of the solid support;

c) functionalizing the activated solid support with one or more cationic groups to form a quaternized solid support, wherein each cationic group is independently a nitrogen-containing cationic group, a phosphorous-containing cationic group, a sulfur-containing cationic group, or any combination thereof;

d) functionalizing the quaternized solid support with one or more acidic groups to form a quaternized-acidic solid support, wherein each acidic group is independently a Bronsted-Lowry acid; and

e) washing the quaternized-acidic solid support with a base to produce any one of the solid-supported catalysts described herein.

In yet another aspect, provided is a method for producing any one of the solid-supported catalysts described herein, by:

a) providing a carbonaceous material;

b) carbonizing at least a portion of the carbonaceous material to form a solid support;

b) activating at least a portion of the solid support;

c) functionalizing the activated solid support with one or more acidic groups to form an acidified solid support, wherein each acidic group is independently a Bronsted-Lowry acid;

d) functionalizing the acidified solid support with one or more cationic groups to form a quaternized-acidic solid support, wherein each cationic group is independently a nitrogen-containing cationic group, a phosphorous-containing cationic group, or a sulfur-containing cationic group; and

e) washing the quaternized-acidic solid support with a base to produce any one of the solid-supported catalysts described herein.

Provided is also a catalyst produced according to any of the methods described above.

### DESCRIPTION OF THE FIGURES

The following description sets forth exemplary compositions, methods, parameters and the like. It should be recognized, however, that such description is not intended as a limitation on the scope of the present disclosure but is instead provided as a description of exemplary embodiments.

FIG. 1 illustrates a portion of an exemplary polymeric catalyst that has a polymeric backbone and side chains.

FIG. 2 illustrates a portion of an exemplary polymeric catalyst, in which a side chain with the basic group is directly connected to the polymeric backbone and in which a side chain with an ionic group is connected to the polymeric backbone by a linker.

FIG. 3 illustrates an ionic group in a portion of an exemplary polymeric catalyst.

FIG. 4A illustrates a portion of an exemplary polymeric catalyst, in which the monomers are randomly arranged in an alternating sequence.

FIG. 4B illustrates a portion of an exemplary polymeric catalyst, in which the monomers are arranged in blocks of monomers, and the block of basic monomers alternates with the block of ionic monomers.

FIGS. 5A and 5B illustrate a portion of exemplary polymeric catalysts with cross-linking within a given polymeric chain.

FIGS. 6A, 6B, 6C and 6D illustrate a portion of exemplary polymeric catalysts with cross-linking between two polymeric chains.

FIG. 7A illustrates a portion of an exemplary polymeric catalyst with a polyethylene backbone.

FIG. 7B illustrates a portion of an exemplary polymeric catalyst with a polyvinylalcohol backbone.

FIG. 7C illustrates a portion of an exemplary polymeric catalyst with a basic backbone.

FIG. 8A illustrates two side chains in an exemplary polymeric catalyst, in which there are three carbon atoms between the side chain with the Bronsted-Lowry base and the side chain with the ionic group.

FIG. 8B illustrates two side chains in another exemplary polymeric catalyst, in which there are zero carbons between the side chain with the Bronsted-Lowry base and the side chain with the ionic group.

FIG. 9A depicts an exemplary reaction to activate a carbon support by introducing a reactive linker by a Friedel-Crafts reaction; and

FIG. 9B depicts an exemplary reaction scheme to prepare a dual-functionalized catalyst from an activated carbon support, in which the catalyst has both basic and ionic moieties.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Described herein are catalysts that can be used as a catalyst to at least partially break down lignin to produce one or more lignin digestion products. The catalysts described herein can also be easily recycled and reused. The ability to recycle and reuse the catalyst presents several advantages, including reducing the cost of converting lignocellulose into industrially important chemicals.

### DEFINITIONS

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

Reference to “about” a value or parameter herein includes (and describes) embodiments that are directed to that value or parameter per se. For example, description referring to “about x” includes description of “x” per se. In other instances, the term “about” when used in association with other measurements, or used to modify a value, a unit, a constant, or a range of values, refers to variations of between ±0.1% and ±15% of the stated number. For example, in one variation, “about 1” refers to a range between 0.85 and 1.15.

Reference to “between” two values or parameters herein includes (and describes) embodiments that include those two values or parameters per se. For example, description referring to “between x and y” includes description of “x” and “y” per se.

“Bronsted-Lowry base” refers to a molecule, or substituent thereof, in neutral or ionic form that is capable of donating OH⁻.

“Homopolymer” refers to a polymer having at least two monomer units, and where all the units contained within the polymer are derived from the same monomer. One suitable example is polyethylene, where ethylene monomers are linked to form a uniform repeating chain (—CH₂—CH₂—CH₂—). Another suitable example is polyvinyl chloride, having a structure (—CH₂—CHCl—CH₂—CHCl—) where the —CH₂—CHCl— repeating unit is derived from the H₂C═CHCl monomer.

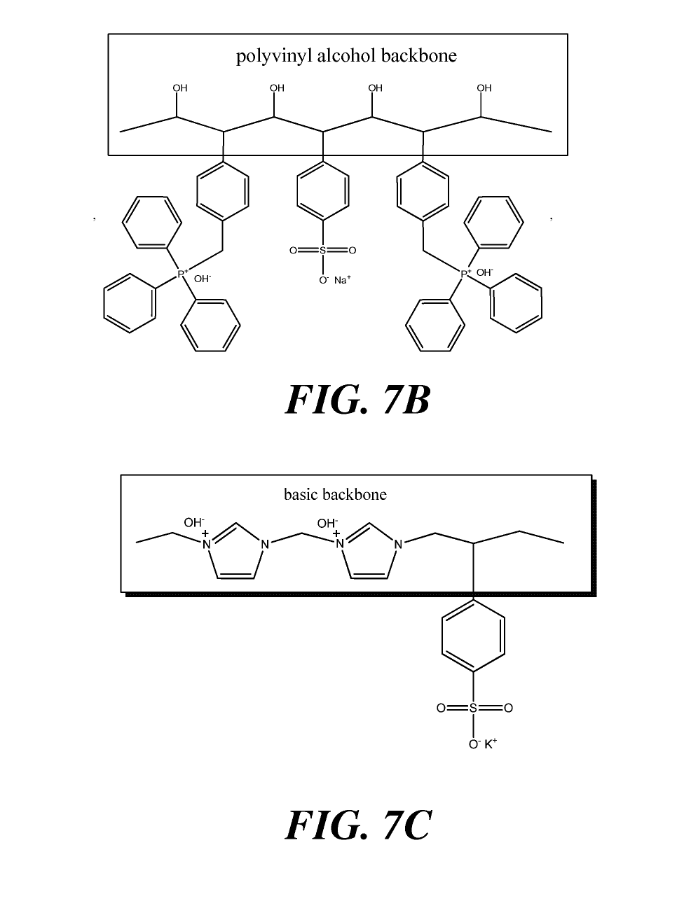

“Heteropolymer” refers to a polymer having at least two monomer units, and where at least one monomeric unit differs from the other monomeric units in the polymer. Heteropolymer also refers to polymers having difunctionalized or trifunctionalized monomer units that can be incorporated in the polymer in different ways. The different monomer units in the polymer can be in a random order, in an alternating sequence of any length of a given monomer, or in blocks of monomers. One suitable example is polyethyleneimidazolium, where if in an alternating sequence, would be the polymer depicted in FIG. 7C. Another suitable example is polystyrene-co-divinylbenzene, where if in an alternating sequence, could be (—CH₂—CH(phenyl)-CH₂—CH(4-ethylenephenyl)-CH₂—CH(phenyl)-CH₂—CH(4-ethylenephenyl)-). Here, the ethenyl functionality could be at the 2, 3, or 4 position on the phenyl ring.

As used herein, [symbol] denotes the attachment point of a moiety to the parent structure.

When a range of values is listed, it is intended to encompass each value and sub-range within the range. For example, “C₁₋₆ alkyl” (which may also be referred to as 1-6C alkyl, C1-C6 alkyl, or C1-6 alkyl) is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” includes saturated straight-chained or branched monovalent hydrocarbon radicals, which contain only C and H when unsubstituted. In some embodiments, alkyl as used herein may have 1 to 10 carbon atoms (e.g., C₁₋₁₀ alkyl), 1 to 6 carbon atoms (e.g., C₁₋₆ alkyl), or 1 to 3 carbon atoms (e.g., C₁₋₃ alkyl). Representative straight-chained alkyls include, for example, methyl, ethyl, n-propyl, n-butyl, n-pentyl, and n-hexyl. Representative branched alkyls include, for example, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, and 2,3-dimethylbutyl. When an alkyl residue having a specific number of carbons is named, all geometric isomers having that number of carbons are intended to be encompassed and described; thus, for example, “butyl” is meant to include n-butyl, sec-butyl, iso-butyl, and tert-butyl; “propyl” includes n-propyl, and iso-propyl.

“Alkoxy” refers to the group —O-alkyl, which is attached to the parent structure through an oxygen atom. Examples of alkoxy may include methoxy, ethoxy, propoxy, and isopropoxy. In some embodiments, alkoxy as used herein has 1 to 6 carbon atoms (e.g., O—(C₁₋₆ alkyl)), or 1 to 4 carbon atoms (e.g., O—(C₁₋₄ alkyl)).

“Alkenyl” refers to straight-chained or branched monovalent hydrocarbon radicals, which contain only C and H when unsubstituted and at least one double bond. In some embodiments, alkenyl has 2 to 10 carbon atoms (e.g., C₂₋₁₀ alkenyl), or 2 to 5 carbon atoms (e.g., C₂₋₅ alkenyl). When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl may include —CH═CH₂, —CH₂—CH═CH₂ and —CH₂—CH═CH—CH═CH₂. The one or more carbon-carbon double bonds can be internal (such as in 2-butenyl) or terminal (such as in 1-butenyl). Examples of C₂₋₄ alkenyl groups include ethenyl (C2), 1-propenyl (C3), 2-propenyl (C3), 1-butenyl (C4), 2-butenyl (C4), and butadienyl (C4). Examples of C₂₋₆ alkenyl groups include the aforementioned C₂₋₄ alkenyl groups as well as pentenyl (C5), pentadienyl (C5), and hexenyl (C6). Additional examples of alkenyl include heptenyl (C7), octenyl (C8), and octatrienyl (C8).

“Alkynyl” refers to straight-chained or branched monovalent hydrocarbon radicals, which contain only C and H when unsubstituted and at least one triple bond. In some embodiments, alkynyl has 2 to 10 carbon atoms (e.g., C₂₋₁₀ alkynyl), or 2 to 5 carbon atoms (e.g., C₂₋₅ alkynyl). When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl may include —C≡CH or —C≡C—CH₃.

In some embodiments, alkyl, alkoxy, alkenyl, and alkynyl at each occurrence may independently be unsubstituted or substituted by one or more of substituents. In certain embodiments, substituted alkyl, substituted alkoxy, substituted alkenyl, and substituted alkynyl at each occurrence may independently have 1 to 5 substituents, 1 to 3 substituents, 1 to 2 substituents, or 1 substituent. Examples of alkyl, alkoxy, alkenyl, and alkynyl substituents may include alkoxy, cycloalkyl, aryl, aryloxy, amino, amido, carbamate, carbonyl, oxo (═O), heteroalkyl (e.g., ether), heteroaryl, heterocycloalkyl, cyano, halo, haloalkoxy, haloalkyl, and thio. In certain embodiments, the one or more substituents of substituted alkyl, alkoxy, alkenyl, and alkynyl is independently selected from cycloalkyl, aryl, heteroalkyl (e.g., ether), heteroaryl, heterocycloalkyl, cyano, halo, haloalkoxy, haloalkyl, oxo, —ORₐ, —N(Rₐ)(R_{b}), —C(O)N(Rₐ)(R_{b}), —N(Rₐ)C(O)R_{b}, —C(O)Rₐ, —N(Rₐ)S(O)ₜRₐ (where t is 1 or 2), —SRₐ, and —S(O)ₜN(Rₐ)(R_{b}) (where t is 1 or 2). In certain embodiments, each Rₐ is independently hydrogen, alkyl, alkenyl, alkynyl, haloalkyl, heteroalkyl, cycloalkyl, aryl, heterocycloalkyl, heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl. In one embodiment, Rₐ is independently hydrogen, alkyl, haloalkyl, cycloalkyl, aryl, aralkyl (e.g., alkyl substituted with aryl, bonded to parent structure through the alkyl group), heterocycloalkyl, or heteroaryl.

“Heteroalkyl”, “heteroalkenyl” and “heteroalkynyl” includes alkyl, alkenyl and alkynyl groups, respectively, wherein one or more skeletal chain atoms are selected from an atom other than carbon, e.g., oxygen, nitrogen, sulfur, phosphorus, or any combinations thereof. For example, heteroalkyl may be an ether where at least one of the carbon atoms in the alkyl group is replaced with an oxygen atom. A numerical range can be given, e.g., C₁₋₄ heteroalkyl which refers to the chain length in total, which in this example is 4 atoms long. For example, a —CH₂OCH₂CH₃ group is referred to as a “C₄” heteroalkyl, which includes the heteroatom center in the atom chain length description. Connection to the rest of the parent structure can be through, in one embodiment, a heteroatom, or, in another embodiment, a carbon atom in the heteroalkyl chain. Heteroalkyl groups may include, for example, ethers such as methoxyethanyl (—CH₂CH₂OCH₃), ethoxymethanyl (—CH₂OCH₂CH₃), (methoxymethoxy)ethanyl (—CH₂CH₂OCH₂OCH₃), (methoxymethoxy)methanyl (—CH₂OCH₂OCH₃) and (methoxyethoxy)methanyl (—CH₂OCH₂CH₂OCH₃); amines such as —CH₂CH₂NHCH₃, —CH₂CH₂N(CH₃)₂, —CH₂NHCH₂CH₃, and —CH₂N(CH₂CH₃)(CH₃). In some embodiments, heteroalkyl, heteroalkenyl, or heteroalkynyl may be unsubstituted or substituted by one or more of substituents. In certain embodiments, a substituted heteroalkyl, heteroalkenyl, or heteroalkynyl may have 1 to 5 substituents, 1 to 3 substituents, 1 to 2 substituents, or 1 substituent. Examples for heteroalkyl, heteroalkenyl, or heteroalkynyl substituents may include the substituents described above for alkyl.

“Carbocyclyl” may include cycloalkyl, cycloalkenyl or cycloalkynyl. “Cycloalkyl” refers to a monocyclic or polycyclic alkyl group. “Cycloalkenyl” refers to a monocyclic or polycyclic alkenyl group (e.g., containing at least one double bond). “Cycloalkynyl” refers to a monocyclic or polycyclic alkynyl group (e.g., containing at least one triple bond). The cycloalkyl, cycloalkenyl, or cycloalkynyl can consist of one ring, such as cyclohexyl, or multiple rings, such as adamantyl. A cycloalkyl, cycloalkenyl, or cycloalkynyl with more than one ring can be fused, spiro or bridged, or combinations thereof. In some embodiments, cycloalkyl, cycloalkenyl, and cycloalkynyl has 3 to 10 ring atoms (i.e., C₃-C₁₀ cycloalkyl, C₃-C₁₀ cycloalkenyl, and C₃-C₁₀ cycloalkynyl), 3 to 8 ring atoms (e.g., C₃-C₈ cycloalkyl, C₃-C₈ cycloalkenyl, and C₃-C₈ cycloalkynyl), or 3 to 5 ring atoms (i.e., C₃-C₅ cycloalkyl, C₃-C₅ cycloalkenyl, and C₃-C₅ cycloalkynyl). In certain embodiments, cycloalkyl, cycloalkenyl, or cycloalkynyl includes bridged and spiro-fused cyclic structures containing no heteroatoms. In other embodiments, cycloalkyl, cycloalkenyl, or cycloalkynyl includes monocyclic or fused-ring polycyclic (i.e., rings which share adjacent pairs of ring atoms) groups. C₃₋₆ carbocyclyl groups may include, for example, cyclopropyl (C₃), cyclobutyl (C₄), cyclopentyl (C₅), cyclopentenyl (C₅), cyclohexyl (C₆), cyclohexenyl (C₆), and cyclohexadienyl (C₆). C₃₋₈ carbocyclyl groups may include, for example, the aforementioned C₃₋₆ carbocyclyl groups as well as cycloheptyl (C₇), cycloheptadienyl (C₇), cycloheptatrienyl (C₇), cyclooctyl (C₈), bicyclo[2.2.1]heptanyl, and bicyclo[2.2.2]octanyl. C₃₋₁₀ carbocyclyl groups may include, for example, the aforementioned C₃₋₈ carbocyclyl groups as well as octahydro-1H-indenyl, decahydronaphthalenyl, and spiro[4.5]decanyl.

“Heterocyclyl” refers to carbocyclyl as described above, with one or more ring heteroatoms independently selected from nitrogen, oxygen, phosphorous, and sulfur. Heterocyclyl may include, for example, heterocycloalkyl, heterocycloalkenyl, and heterocycloalknyl. In some embodiments, heterocyclyl is a 3- to 18-membered non-aromatic monocyclic or polycyclic moiety that has at least one heteroatom selected from nitrogen, oxygen, phosphorous and sulfur. In certain embodiments, the heterocyclyl can be a monocyclic or polycyclic (e.g., bicyclic, tricyclic or tetracyclic), wherein polycyclic ring systems can be a fused, bridged or spiro ring system. Heterocyclyl polycyclic ring systems can include one or more heteroatoms in one or both rings.

An N-containing heterocyclyl moiety refers to an non-aromatic group in which at least one of the skeletal atoms of the ring is a nitrogen atom. The heteroatom(s) in the heterocyclyl group is optionally oxidized. One or more nitrogen atoms, if present, are optionally quaternized. In certain embodiments, heterocyclyl may also include ring systems substituted with one or more oxide (—O—) substituents, such as piperidinyl N-oxides. The heterocyclyl is attached to the parent structure through any atom of the ring(s).

In some embodiments, heterocyclyl also includes ring systems with one or more fused carbocyclyl, aryl or heteroaryl groups, wherein the point of attachment is either on the carbocyclyl or heterocyclyl ring. In some embodiments, heterocyclyl is a 5-10 membered non-aromatic ring system having ring carbon atoms and 1-4 ring heteroatoms, wherein each heteroatom is independently selected from nitrogen, oxygen and sulfur (e.g., 5-10 membered heterocyclyl). In some embodiments, a heterocyclyl group is a 5-8 membered non-aromatic ring system having ring carbon atoms and 1-4 ring heteroatoms, wherein each heteroatom is independently selected from nitrogen, oxygen and sulfur (e.g., 5-8 membered heterocyclyl). In some embodiments, a heterocyclyl group is a 5-6 membered non-aromatic ring system having ring carbon atoms and 1-4 ring heteroatoms, wherein each heteroatom is independently selected from nitrogen, oxygen and sulfur (e.g., 5-6 membered heterocyclyl). In some embodiments, the 5-6 membered heterocyclyl has 1-3 ring heteroatoms selected from nitrogen, oxygen and sulfur. In some embodiments, the 5-6 membered heterocyclyl has 1-2 ring heteroatoms selected from nitrogen, oxygen and sulfur. In some embodiments, the 5-6 membered heterocyclyl has 1 ring heteroatom selected from nitrogen, oxygen and sulfur.

Exemplary 3-membered heterocyclyls containing 1 heteroatom may include azirdinyl, oxiranyl, thiorenyl. Exemplary 4-membered heterocyclyls containing 1 heteroatom may include azetidinyl, oxetanyl and thietanyl. Exemplary 5-membered heterocyclyls containing 1 heteroatom may include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyls containing 2 heteroatoms may include dioxolanyl, oxathiolanyl and dithiolanyl. Exemplary 5-membered heterocyclyls containing 3 heteroatoms may include triazolinyl, oxadiazolinyl, and thiadiazolinyl. Exemplary 6-membered heterocyclyl groups containing 1 heteroatom may include piperidinyl, tetrahydropyranyl, dihydropyridinyl, and thianyl. Exemplary 6-membered heterocyclyl groups containing 2 heteroatoms may include piperazinyl, morpholinyl, dithianyl, dioxanyl. Exemplary 6-membered heterocyclyl groups containing 2 heteroatoms may include triazinanyl. Exemplary 7-membered heterocyclyl groups containing 1 heteroatom may include azepanyl, oxepanyl and thiepanyl. Exemplary 8-membered heterocyclyl groups containing 1 heteroatom may include azocanyl, oxecanyl and thiocanyl. Exemplary bicyclic heterocyclyl groups may include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydro-pyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetra-hydrofuro[3,2-c]pyridinyl, 4,5,6,7-tetrahydrothieno[3,2-b]pyridinyl, and 1,2,3,4-tetrahydro-1,6-naphthyridinyl.

“Aryl” refers to an aromatic group having a single ring (e.g., phenyl), multiple rings (e.g., biphenyl), or multiple fused rings (e.g., naphthyl, fluorenyl, and anthryl). In some embodiments, aryl as used herein has 6 to 10 ring atoms (e.g., C₆-C₁₀ aromatic or C₆-C₁₀ aryl) which has at least one ring having a conjugated pi electron system. For example, bivalent radicals formed from substituted benzene derivatives and having the free valences at ring atoms are named as substituted phenylene radicals. In certain embodiments, aryl may have more than one ring where at least one ring is non-aromatic can be connected to the parent structure at either an aromatic ring position or at a non-aromatic ring position. In certain embodiments, aryl includes monocyclic or fused-ring polycyclic (i.e., rings which share adjacent pairs of ring atoms) groups.

“Heteroaryl” refers to an aromatic group having a single ring, multiple rings, or multiple fused rings, with one or more ring heteroatoms independently selected from nitrogen, oxygen, phosphorous, and sulfur. In some embodiments, heteroaryl is an aromatic, monocyclic or bicyclic ring containing one or more heteroatoms independently selected from nitrogen, oxygen and sulfur with the remaining ring atoms being carbon. In certain embodiments, heteroaryl is a 5- to 18-membered monocyclic or polycyclic (e.g., bicyclic or tricyclic) aromatic ring system (e.g., having 6, 10 or 14 pi electrons shared in a cyclic array) having ring carbon atoms and 1 to 6 ring heteroatoms provided in the aromatic ring system, wherein each heteroatom is independently selected from nitrogen, oxygen, phosphorous and sulfur (e.g., 5-18 membered heteroaryl). In certain embodiments, heteroaryl may have a single ring (e.g., pyridyl, pyridinyl, imidazolyl) or multiple condensed rings (e.g., indolizinyl, benzothienyl) which condensed rings may or may not be aromatic. In other embodiments, heteroaryl may have more than one ring where at least one ring is non-aromatic can be connected to the parent structure at either an aromatic ring position or at a non-aromatic ring position. In one embodiment, heteroaryl may have more than one ring where at least one ring is non-aromatic is connected to the parent structure at an aromatic ring position. Heteroaryl polycyclic ring systems can include one or more heteroatoms in one or both rings.

For example, in one embodiment, an N-containing “heteroaryl” refers to an aromatic group in which at least one of the skeletal atoms of the ring is a nitrogen atom. One or more heteroatom(s) in the heteroaryl group can be optionally oxidized. One or more nitrogen atoms, if present, are optionally quaternized. In other embodiments, heteroaryl may include ring systems substituted with one or more oxide (—O—) substituents, such as pyridinyl N-oxides. The heteroaryl may be attached to the parent structure through any atom of the ring(s).

In other embodiments, heteroaryl may include ring systems with one or more fused aryl groups, wherein the point of attachment is either on the aryl or on the heteroaryl ring. In yet other embodiments, heteroaryl may include ring systems with one or more carbocycyl or heterocycyl groups wherein the point of attachment is on the heteroaryl ring. For polycyclic heteroaryl groups wherein one ring does not contain a heteroatom (e.g., indolyl, quinolinyl, and carbazolyl) the point of attachment can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-10 membered heteroaryl). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-8 membered heteroaryl). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-6 membered heteroaryl). In some embodiments, the 5-6 membered heteroaryl has 1-3 ring heteroatoms selected from nitrogen, oxygen, phosphorous, and sulfur. In some embodiments, the 5-6 membered heteroaryl has 1-2 ring heteroatoms selected from nitrogen, oxygen, phosphorous, and sulfur. In some embodiments, the 5-6 membered heteroaryl has 1 ring heteroatom selected from nitrogen, oxygen, phosphorous, and sulfur.

Examples of heteroaryls may include azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl).

In some embodiments, carbocyclyl (including, for example, cycloalkyl, cycloalkenyl or cycloalkynyl), aryl, heteroaryl, and heterocyclyl at each occurrence may independently be unsubstituted or substituted by one or more of substituents. In certain embodiments, a substituted carbocyclyl (including, for example, substituted cycloalkyl, substituted cycloalkenyl or substituted cycloalkynyl), substituted aryl, substituted heteroaryl, substituted heterocyclyl at each occurrence may be independently may independently have 1 to 5 substituents, 1 to 3 substituents, 1 to 2 substituents, or 1 substituent. Examples of carbocyclyl (including, for example, cycloalkyl, cycloalkenyl or cycloalkynyl), aryl, heteroaryl, heterocyclyl substituents may include alkyl alkenyl, alkoxy, cycloalkyl, aryl, heteroalkyl (e.g., ether), heteroaryl, heterocycloalkyl, cyano, halo, haloalkoxy, haloalkyl, oxo (═O), —ORₐ, —N(Rₐ)(R_{b}), —C(O)N(Rₐ)(R_{b}), —N(Rₐ)C(O)R_{b}, —C(O)Rₐ, —N(Rₐ)S(O)ₜR_{b} (where t is 1 or 2), —SRₐ, and —S(O)ₜN(Rₐ)(R_{b}) (where t is 1 or 2), wherein Rₐ and R_{b} (as applicable) is as described herein.

It should be understood that, as used herein, any moiety referred to as a “linker” refers to the moiety has having bivalency. Thus, for example, “alkyl linker” refers to the same residues as alkyl, but having bivalency. Examples of alkyl linkers include —CH₂—, —CH₂CH₂—, —CH₂CH₂CH₂—, and —CH₂CH₂CH₂CH₂—. “Alkenyl linker” refers to the same residues as alkenyl, but having bivalency. Examples of alkenyl linkers include —CH═CH—, —CH₂—CH═CH— and —CH₂—CH═CH—CH₂—. “Alkynyl linker” refers to the same residues as alkynyl, but having bivalency. Examples alkynyl linkers include —C≡C— or —C≡C—CH₂—. Similarly, “carbocyclyl linker”, “aryl linker”, “heteroaryl linker”, and “heterocyclyl linker” refer to the same residues as carbocyclyl, aryl, heteroaryl, and heterocyclyl, respectively, but having bivalency.

Further, “alkyl carbamate linker” refers to an alkyl linker, in which one or more of the methylene units of the alkyl linker has been replaced with a carbamate moiety. Examples of alkyl carbamate linkers include —CH₂—C(O)—O—NRₐ—CH₂— and —CH₂CH₂O—C(O)—NRₐ—CH₂—, where Rₐ is as described herein.

“Alkyl ester linker” refers to an alkyl linker, in which one or more of the methylene units of the alkyl linker has been replaced with an ester moiety (—C(O)—O— or —O—C(O)—). Examples of alkyl ester linkers include —CH₂—C(O)—O—CH₂— and —CH₂CH₂O—C(O)—CH₂—.

“Alkyl ether linker” refers to an alkyl linker, in which one or more of the methylene units of the alkyl liker has been replaced with an ether moiety (—C(O)—). Examples of alkyl esther linkers include —CH₂—C(O)—CH₂— and —CH₂CH₂—C(O)—CH₂—.

“Amino” or “amine” refers to —N(Rₐ)(R_{b}), where each Rₐ and R_{b} is independently selected from hydrogen, alkyl, alkenyl, alkynyl, haloalkyl, heteroalkyl (e.g., bonded through a chain carbon), cycloalkyl, aryl, heterocycloalkyl (e.g., bonded through a ring carbon), heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl. It should be understood that, in one embodiment, amino includes amido (e.g., —NRₐC(O)R_{b}). It should be further understood that in certain embodiments, the alkyl, alkenyl, alkynyl, haloalkyl, heteroalkyl, cycloalkyl, aryl, heterocycloalkyl, or heteroaryl moiety of Rₐ and R_{b} may be further substituted as described herein. Rₐ and R_{b} may be the same or different. For example, in one embodiment, amino is —NH₂ (where Rₐ and R_{b} are each hydrogen). In other embodiments where Rₐ and R_{b} are other than hydrogen, Rₐ and R_{b} can be combined with the nitrogen atom to which they are attached to form a 3-, 4-, 5-, 6-, or 7-membered ring. Such examples may include 1-pyrrolidinyl and 4-morpholinyl.

“Ammonium” refers to —N(Rₐ)(R_{b})(R_{c})⁺, where each Rₐ, R_{b} and R_{c} is independently selected from hydrogen, alkyl, alkenyl, alkynyl, haloalkyl, heteroalkyl (e.g., bonded through a chain carbon), cycloalkyl, aryl, heterocycloalkyl (e.g., bonded through a ring carbon), heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl; or any two of Rₐ, R_{b} and R_{c} may be taken together with the atom to which they are attached to form a cycloalkyl, heterocycloalkyl; or any three of Rₐ, R_{b} and R_{c} may be taken together with the atom to which they are attached to form aryl or heteroaryl. It should be further understood that in certain embodiments, the alkyl, alkenyl, alkynyl, haloalkyl, heteroalkyl, cycloalkyl, aryl, heterocycloalkyl, or heteroaryl moiety of any one or more of Rₐ, R_{b} and R_{c} may be further substituted as described herein. Rₐ, R_{b} and R_{c} may be the same or different.

In certain embodiments, “amino” also refers to N-oxides of the groups —N⁺(H)(Rₐ)O⁻, and —N⁺(Rₐ)(R_{b})O—, where Rₐ and R_{b} are as described herein, where the N-oxide is bonded to the parent structure through the N atom. N-oxides can be prepared by treatment of the corresponding amino group with, for example, hydrogen peroxide or m-chloroperoxybenzoic acid. The person skilled in the art is familiar with reaction conditions for carrying out the N-oxidation.

“Amide” or “amido” refers to a chemical moiety with formula —C(O) N(Rₐ)(R_{b}) or —NR^{a}C(O)R_{b}, where Rₐ and R_{b} at each occurrence are as described herein. In some embodiments, amido is a C₁₋₄ amido, which includes the amide carbonyl in the total number of carbons in the group. When a —C(O)N(Rₐ)(R_{b}) has Rₐ and R_{b} other than hydrogen, they can be combined with the nitrogen atom to form a 3-, 4-, 5-, 6-, or 7-membered ring.

“Carbonyl” refers to —C(O)Rₐ, where Rₐ is hydrogen, alkyl, alkenyl, alkynyl, haloalkyl, heteroalkyl, cycloalkyl, aryl, heterocycloalkyl, heteroaryl, —N(R′)₂, —S(O)ₜR′, where each R′ is independently hydrogen, alkyl, alkenyl, alkynyl, haloalkyl, heteroalkyl, cycloalkyl, aryl, heterocycloalkyl, or heteroaryl, and t is 1 or 2. In certain embodiments where each R′ are other than hydrogen, the two R′ moieties can be combined with the nitrogen atom to which they are attached to form a 3-, 4-, 5-, 6-, or 7-membered ring. It should be understood that, in one embodiment, carbonyl includes amido (e.g., —C(O) N(Rₐ)(R_{b})).

“Carbamate” refers to any of the following groups: —O—C(═O)—N(Rₐ)(R_{b}) and —N(Rₐ)—C(═O)—OR_{b}, wherein Rₐ and R_{b} at each occurrence are as described herein.

“Cyano” refers to a —CN group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy moieties as described above, wherein one or more hydrogen atoms are replaced by halo. For example, where a residue is substituted with more than one halo groups, it may be referred to by using a prefix corresponding to the number of halo groups attached. For example, dihaloaryl, dihaloalkyl, and trihaloaryl refer to aryl and alkyl substituted with two (“di”) or three (“tri”) halo groups, which may be, but are not necessarily, the same halogen; thus, for example, 3,5-difluorophenyl, 3-chloro-5-fluorophenyl, 4-chloro-3-fluorophenyl, and 3,5-difluoro-4-chlorophenyl is within the scope of dihaloaryl. Other examples of a haloalkyl group include difluoromethyl (—CHF₂), trifluoromethyl (—CF₃), 2,2,2-trifluoroethyl, and 1-fluoromethyl-2-fluoroethyl. Each of the alkyl, alkenyl, alkynyl and alkoxy groups of haloalkyl, haloalkenyl, haloalkynyl and haloalkoxy, respectively, can be optionally substituted as defined herein. “Perhaloalkyl” refers to an alkyl or alkylene group in which all of the hydrogen atoms have been replaced with a halogen (e.g., fluoro, chloro, bromo, or iodo). In some embodiments, all of the hydrogen atoms are each replaced with fluoro. In some embodiments, all of the hydrogen atoms are each replaced with chloro. Examples of perhaloalkyl groups include —CF₃, —CF₂CF₃, —CF₂CF₂CF₃, —CCl₃, —CFCl₂, and —CF₂Cl.

“Thio” refers to —SRₐ, wherein Rₐ is as described herein. “Thiol” refers to the group —RₐSH, wherein Rₐ is as described herein.

“Sulfinyl” refers to —S(O)Rₐ. In some embodiments, sulfinyl is —S(O)N(Rₐ)(R_{b}). “Sulfonyl” refers to the —S(O₂)Rₐ. In some embodiments, sulfonyl is —S(O₂) N(Rₐ)(R_{b}) or —S(O₂)OH. For each of these moieties, it should be understood that Rₐ and R_{b} are as described herein.

“Moiety” refers to a specific segment or functional group of a molecule. Chemical moieties are often recognized chemical entities embedded in or appended to a molecule.

As used herein, the term “unsubstituted” means that for carbon atoms, only hydrogen atoms are present besides those valencies linking the atom to the parent molecular group. One example is propyl (—CH₂—CH₂—CH₃). For nitrogen atoms, valencies not linking the atom to the parent molecular group are either hydrogen or an electron pair. For sulfur atoms, valencies not linking the atom to the parent molecular group are either hydrogen, oxygen or electron pair(s).

As used herein, the term “substituted” or “substitution” means that at least one hydrogen present on a group (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. Unless otherwise indicated, a “substituted” group can have a substituent at one or more substitutable positions of the group, and when more than one position in any given structure is substituted, the substituent is either the same or different at each position. Substituents include one or more group(s) individually and independently selected from alkyl alkenyl, alkoxy, cycloalkyl, aryl, heteroalkyl (e.g., ether), heteroaryl, heterocycloalkyl, cyano, halo, haloalkoxy, haloalkyl, oxo (═O), —ORₐ, —N(Rₐ)(R_{b}), —C(O)N(Rₐ)(R_{b}), —N(Rₐ)C(O)R_{b}, —C(O)Rₐ, —N(Rₐ)S(O)ₜR_{b} (where t is 1 or 2), —SRₐ, and —S(O)ₜN(Rₐ)(R_{b}) (where t is 1 or 2), wherein Rₐ and R_{b} (as applicable) is as described herein.

Where substituent groups are specified by their conventional chemical formulae, written from left to right, they equally encompass the chemically identical substituents that would result from writing the structure from right to left, e.g., —CH₂O— is equivalent to —OCH₂—.

### Polymeric and Solid-Supported Catalysts

The catalysts described herein may include polyemeric catalysts and solid-supported catalysts.

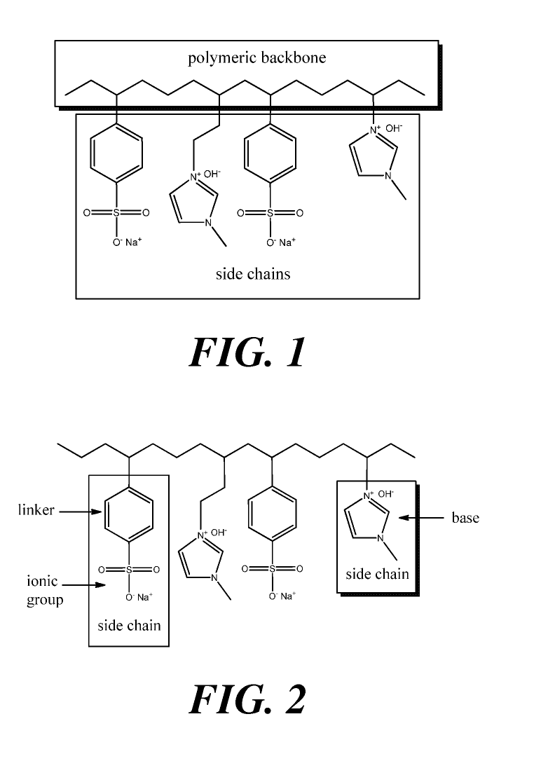

In one aspect, the catalyst is a polymer made up of basic monomers and ionic monomers (which are also referred to as “ionomers”) connected to form a polymeric backbone. Each basic monomer independently includes at least one Bronsted-Lowry base with at least one nitrogen-containing cationic group, at least one phosphorous-containing cationic group, at least one sulfur-containing cationic group, or any combinations thereof. Each ionic monomer independently includes one or more anionic groups and one or more counterions. In certain embodiments of the polymeric catalyst, at least some of the basic and ionic monomers may independently include a linker connecting the Bronsted-Lowry base or the anionic group (as applicable) to a portion of the polymeric backbone. For the basic monomers, the Bronsted-Lowry base and the linker together form a side chain. Similarly, for the ionic monomers, the anionic group, its counterion, and the linker together form a side chain. With reference to the portion of the exemplary polymer depicted in FIG. 1, the side chains are pendant from the polymeric backbone.

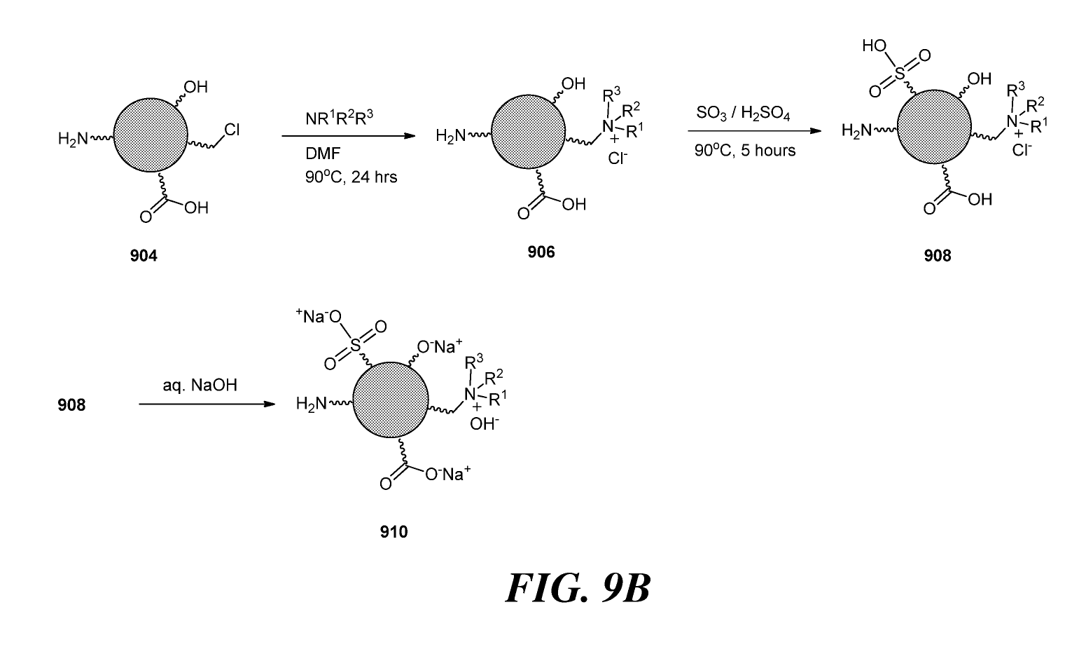

In another aspect, the catalyst is solid-supported, having basic moieties and ionic moieties each attached to a solid support. Each basic moiety independently includes at least one Bronsted-Lowry base with at least one nitrogen-containing cationic group, at least one phosphorous-containing cationic group, at least one sulfur-containing cationic group, or any combinations thereof. Each ionic moiety independently includes one or more anionic groups and one or more counterions. In certain embodiments of the solid-supported catalyst, at least some of the basic and ionic moieties may independently include a linker connecting the Bronsted-Lowry base or the anionic group (as applicable) to the solid support. With reference to FIG. 9B, catalyst 910 is an exemplary solid-supported catalyst with basic and ionic moieties.

a) Basic Monomers and Moieties

The polymeric catalysts include a plurality of basic monomers, where as the solid-supported catalysts includes a plurality of basic moieties attached to a solid support.

In some embodiments, a plurality of basic monomers (e.g., of a polymeric catalyst) or a pluarlity of basic moieties (e.g., of a solid-supported catalyst) has at least one Bronsted-Lowry base. The Bronsted-Lowry base may be on different monomers or on the same monomer.

In some embodiments, the basic monomers (e.g., of a polymeric catalyst) or basic moieties (e.g., of a solid-supported catalyst) may have one Bronsted-Lowry base. In other embodiments, the basic monomers (e.g., of a polymeric catalyst) or basic moieties (e.g., of a solid-supported catalyst) may have two or more Bronsted-Lowry bases, as is chemically feasible. When the basic monomers (e.g., of a polymeric catalyst) or basic moieties (e.g., of a solid-supported catalyst) have two or more Bronsted-Lowry bases, the bases may be the same or different.

Suitable Bronsted-Lowry bases may include any strong Bronsted-Lowry base. In some embodiments, the Bronsted-Lowry bases may have one or more nitrogen-containing cationic groups, one or more phosphorous-containing cationic groups, or one or more sulfur-containing cationic groups. It should be understood that cationic groups of the Bronsted-Lowry base coordinates with one or more anionic groups, such as a hydroxide ion. In certain embodiments, the Bronsted-Lowry base at each occurrence may be independently selected from pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.

The basic monomers (e.g., of a polymeric catalyst) or basic moieties (e.g., of a solid-supported catalyst) may either all have the same Bronsted-Lowry base, or may have different Bronsted-Lowry bases. In an exemplary embodiment, each Bronsted-Lowry base in the catalyst is imidazolium hydroxide. In another exemplary embodiment, each Bronsted-Lowry base in the catalyst is triphenyl phosphonium hydroxide. In yet another exemplary embodiment, the Bronsted-Lowry base in some monomers of the catalyst is imidazolium hydroxide, while the Bronsted-Lowry base in other monomers of the catalyst is triphenyl phosphonium hydroxide.

With reference to the portion of an exemplary polymeric catalyst depicted in FIG. 2, the Bronsted-Lowry base in the side chains of the basic monomers may be directly connected to the polymeric backbone or connected to the polymeric backbone by a linker. Similarly, with reference to the exemplary solid-supported catalyst depicted in FIG. 9B, the Bronsted-Lowry base of the basic moieties may be directly connected to the polymeric backbone or connected to the polymeric backbone by a linker.

Suitable linkers may include, for example, unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate. In some embodiments, the linker is an unsubstituted or substituted C5 or C6 aryl linker. In certain embodiments, the linker is an unsubstituted or substituted phenyl linker. In one exemplary embodiment, the linker is unsubstituted phenyl linker. In another exemplary embodiment, the linker is substituted phenyl linker (e.g., hydroxy-substituted phenyl linker).

In other embodiments, each linker in a basic monomer (e.g., of a polymeric catalyst) or a basic moiety (e.g., of a solid-supported catalyst) is independently selected from:

unsubstituted alkyl linker;

alkyl linker substituted 1 to 5 substituents independently selected from oxo, hydroxy, halo, amino;

unsubstituted cycloalkyl linker;

cycloalkyl linker substituted 1 to 5 substituents independently selected from oxo, hydroxy, halo, amino;

unsubstituted alkenyl linker;

alkenyl linker substituted 1 to 5 substituents independently selected from oxo, hydroxy, halo, amino;

unsubstituted aryl linker;

aryl linker substituted 1 to 5 substituents independently selected from oxo, hydroxy, halo, amino;

unsubstituted heteroaryl linker; or heteroaryl linker substituted 1 to 5 substituents independently selected from oxo, hydroxy, halo, amino.

Further, it should be understood that some or all of the basic monomers connected to the polymeric backbone by a linker may have the same linker, or independently have different linkers.

In some embodiments, each basic monomer (e.g., of a polymeric catalyst) and each basic moiety (e.g., of a solid-supported catalyst) may independently have the structure of Formulas I-VI:

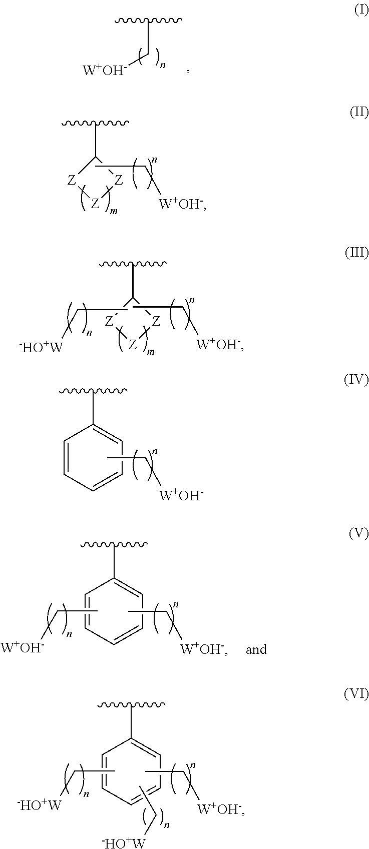

wherein:

each W is independently N(Rₐ)(R_{b})(R_{c}), P(Rₐ)(R_{b})(R_{c}), S(Rₐ)(R_{b}), S(Rₐ)(R_{b})(R_{c}), or S(Rₐ)(R_{b})(R_{c})(R_{d}),
wherein each Rₐ, R_{b}, R_{c}, and R_{d} (if present) is independently hydrogen, alkyl, alkenyl, alkynyl, haloalkyl, heteroalkyl, cycloalkyl, aryl, heterocycloalkyl, heteroaryl, —C(O)R′ or —S(O)ₜR′,
   wherein t is 1 or 2;
   wherein each R′ is independently hydrogen, alkyl, alkenyl, alkynyl, haloalkyl, heteroalkyl, cycloalkyl, aryl, heterocycloalkyl, or heteroaryl; or
any two of Rₐ, R_{b} and R_{c} may be taken together with the atom to which they are attached to form a cycloalkyl or heterocycloalkyl; or
any three of Rₐ, R_{b} and R_{c} may be taken together with the atom to which they are attached to form aryl or heteroaryl;

each Z is independently C(R²)(R³), N(R⁴), S, S(R⁵)(R⁶), S(O)(R⁵)(R⁶), SO₂, or O, wherein any two adjacent Z can (to the extent chemically feasible) be joined by a double bond, or taken together to form a group selected from cycloalkyl, heterocycloalkyl, aryl or heteroaryl;

each m is independently 0, 1, 2, or 3;

each n is independently 0, 1, 2, or 3;

each R², R³, and R⁴ is independently hydrogen, alkyl, heteroalkyl, cycloalkyl, heterocyclyl, aryl, or heteroaryl; and

each R⁵ and R⁶ is independently alkyl, heteroalkyl, cycloalkyl, heterocyclyl, aryl, or heteroaryl.

In some embodiments, each basic monomer (e.g., of a polymeric catalyst) and each basic moiety (e.g., of a solid-supported catalyst) may independently have the structure of Formulas IA, IB, IVA, or IVB. In other embodiments, each basic monomer (e.g., of a polymeric catalyst) and each basic moiety (e.g., of a solid-supported catalyst) may independently have the structure of Formulas IIA, IIB, IIC, IVA, IVB, or IVC. In other embodiments, each basic monomer (e.g., of a polymeric catalyst) and each basic moiety (e.g., of a solid-supported catalyst) may independently have the structure of Formulas IIIA, IIIB, or IIIC. In some embodiments, each basic monomer (e.g., of a polymeric catalyst) and each basic moiety (e.g., of a solid-supported catalyst) may independently have the structure of Formulas VA, VB, or VC. In some embodiments, each basic monomer (e.g., of a polymeric catalyst) and each basic moiety (e.g., of a solid-supported catalyst) may independently have the structure of Formula IA. In other embodiments, each basic monomer (e.g., of a polymeric catalyst) and each basic moiety (e.g., of a solid-supported catalyst) may independently have the structure of Formula IB.

In certain embodiments, each W⁺OH⁻ moiety is [N(Rₐ)(R_{b})(R_{c})]⁺OH⁻. In one variation, Rₐ, R_{b}, and R_{c} are each hydrogen. In another variation, each Rₐ, R_{b}, and R_{c} are independently hydrogen, alkyl, heteroalkyl, cycloalkyl, heterocycloalkyl, aryl, or heteroaryl. In yet another variation, two of Rₐ, R_{b}, and R_{c} are taken together with the atom to which they are attached to form a cycloalkyl or heterocycloalkyl. In yet another variation, Rₐ, R_{b}, and R_{c} are taken together with the atom to which they are attached to form an aryl or heteroaryl.

In certain embodiments, each W⁺OH⁻ moiety is [P(Rₐ)(R_{b})(R_{c})]⁺OH⁻. In one variation, Rₐ, R_{b}, and R_{c} are independently alkyl, heteroalkyl, cycloalkyl, heterocycloalkyl, aryl, heteroaryl, or halo. In one variation, Rₐ, R_{b}, and R_{c} are each independently alkyl. In another variation, Rₐ, R_{b}, and R_{c} are each aryl. In another variation, Rₐ, R_{b}, and R_{c} are each phenyl. In another variation, Rₐ, R_{b}, and R_{c} are each halo.

In certain embodiments, each W⁺OH⁻ moiety is independently [S(Rₐ)(R_{b})]⁺OH⁻, [S(Rₐ)(R_{b})(R_{c})]₊OH₋, or [S(Rₐ)(R_{b})(R_{c})(R_{d})]₊OH₋. In one variation, Rₐ, R_{b}, R_{c}, and R_{d} (if present) and are independently hydrogen, alkyl, heteroalkyl, cycloalkyl, heterocycloalkyl, aryl, or heteroaryl. In one variation, Rₐ, R_{b}, R_{c}, and R_{d} (if present) are each independently hydrogen. In one variation, Rₐ, R_{b}, R_{c}, and R_{d} (if present) are each independently alkyl. In another variation, Rₐ, R_{b}, R_{c}, and R_{d} (if present) are each aryl. In another variation, Rₐ, R_{b}, R_{c}, and R_{d} (if present) are each phenyl.

In some embodiments, Z can be chosen from C(R²)(R³), N(R⁴), SO₂, and O. In some embodiments, any two adjacent Z can be taken together to form a group selected from a heterocycloalkyl, aryl, and heteroaryl. In other embodiments, any two adjacent Z can be joined by a double bond. Any combination of these embodiments is also contemplated (as chemically feasible).

In some embodiments, m is 2 or 3. In other embodiments, n is 1, 2, or 3. In some embodiments, R¹ can be hydrogen, alkyl or heteroalkyl. In some embodiments, R¹ can be hydrogen, methyl, or ethyl. In some embodiments, each R², R³, and R⁴ can independently be hydrogen, alkyl, heterocyclyl, aryl, or heteroaryl. In other embodiments, each R², R³ and R⁴ can independently be heteroalkyl, cycloalkyl, heterocyclyl, or heteroaryl. In some embodiments, each R⁵ and R⁶ can independently be alkyl, heterocyclyl, aryl, or heteroaryl. In another embodiment, any two adjacent Z can be taken together to form cycloalkyl, heterocycloalkyl, aryl or heteroaryl.

In certain embodiments, the basic monomers of the polymeric catalyst may have a side chain with a Bronsted-Lowry base that is connected to the polymeric backbone by a linker. In certain embodiments, the basic moieties of the solid-supported catalyst may have a side chain with a Bronsted-Lowry base that is attached to the solid support by a linker. Side chains with one or more Bronsted-Lowry bases connected by a linker may include, for example,

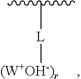

wherein:

W is as defined for Formulas I-VI;

L is an unsubstituted alkyl linker, alkyl linker substituted with oxo, unsubstituted cycloalkyl, unsubstituted aryl, unsubstituted heterocycloalkyl, and unsubstituted heteroaryl; and

r is an integer.

In certain embodiments, r is 1, 2, 3, 4, or 5 (as applicable or chemically feasible).

In some embodiments, at least some of the basic side chains (e.g., of a polymeric catalyst) and at least some of the basic moieties (e.g., of a solid-supported catalyst) may be:

wherein:

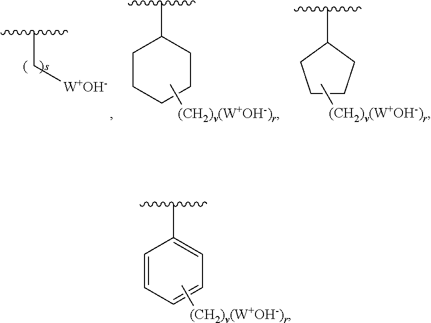

W is as defined for Formulas I-VI;

s is 1 to 10;

each r is independently 1, 2, 3, 4, or 5 (as applicable or chemically feasible); and

v is 0 to 10.

In certain embodiments, s is 1 to 9, or 1 to 8, or 1 to 7, or 1 to 6, or 1 to 5, or 1 to 4, or 1 to 3, or 2, or 1. In certain embodiments, w is 0 to 9, or 0 to 8, or 0 to 7, or 0 to 6, or 0 to 5, or 0 to 4, or 0 to 3, or 0 to 2, 1or 0).

In some embodiments, at least some of the basic side chains (e.g., of a polymeric catalyst) and at least some of the basic moieties (e.g., of a solid-supported catalyst) may be:

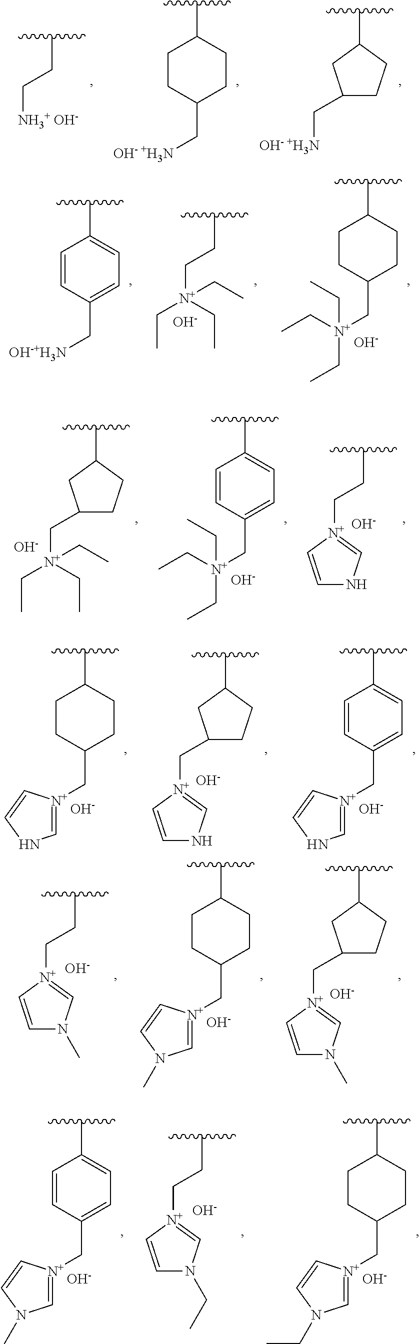

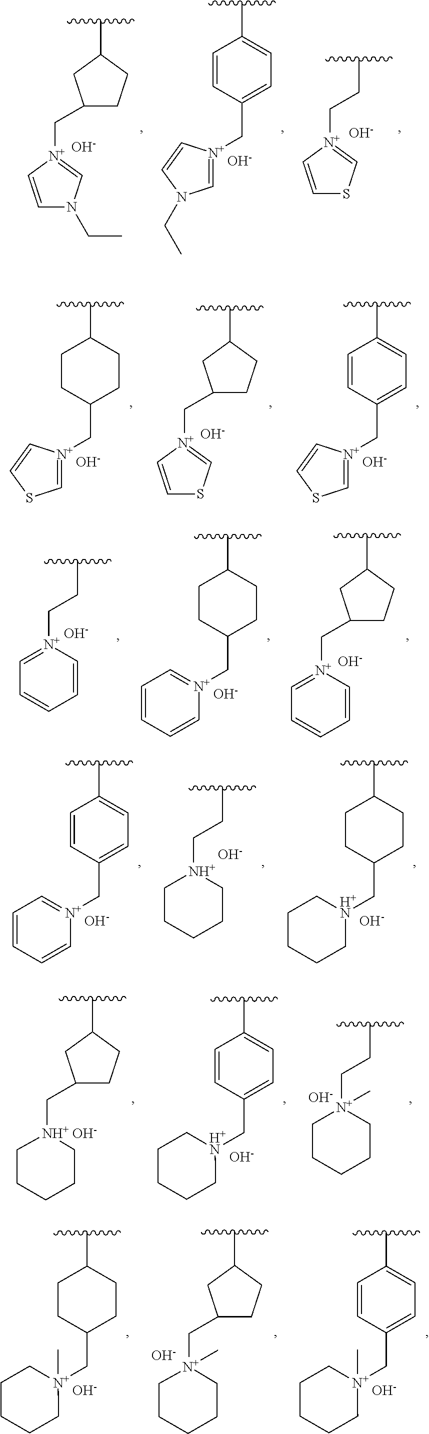

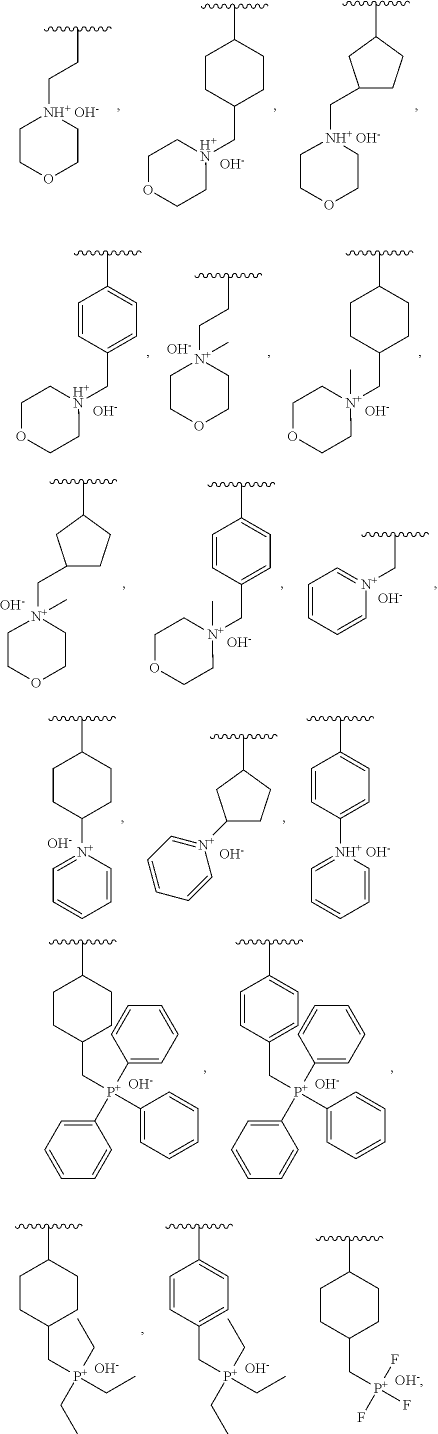

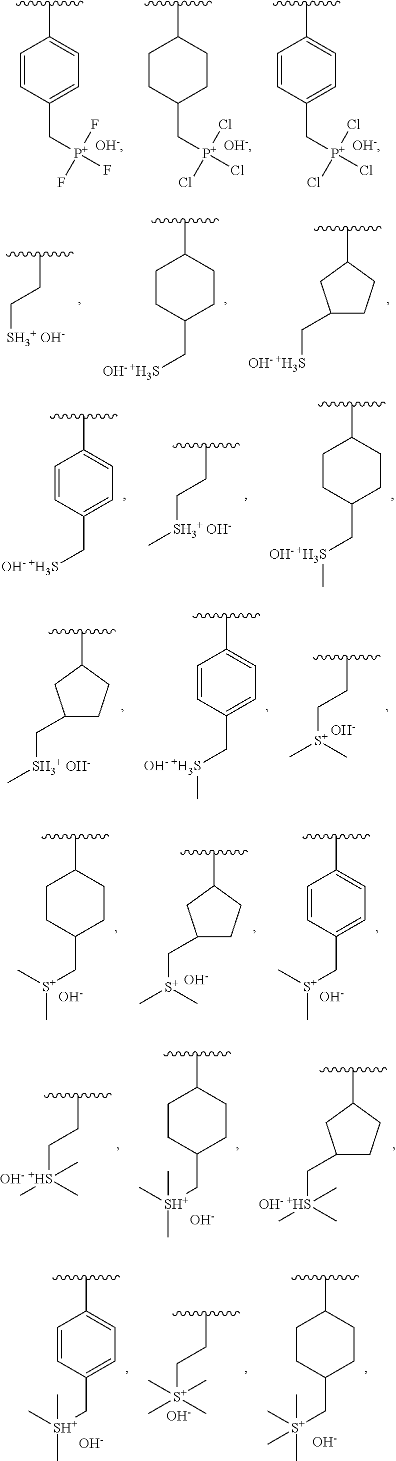

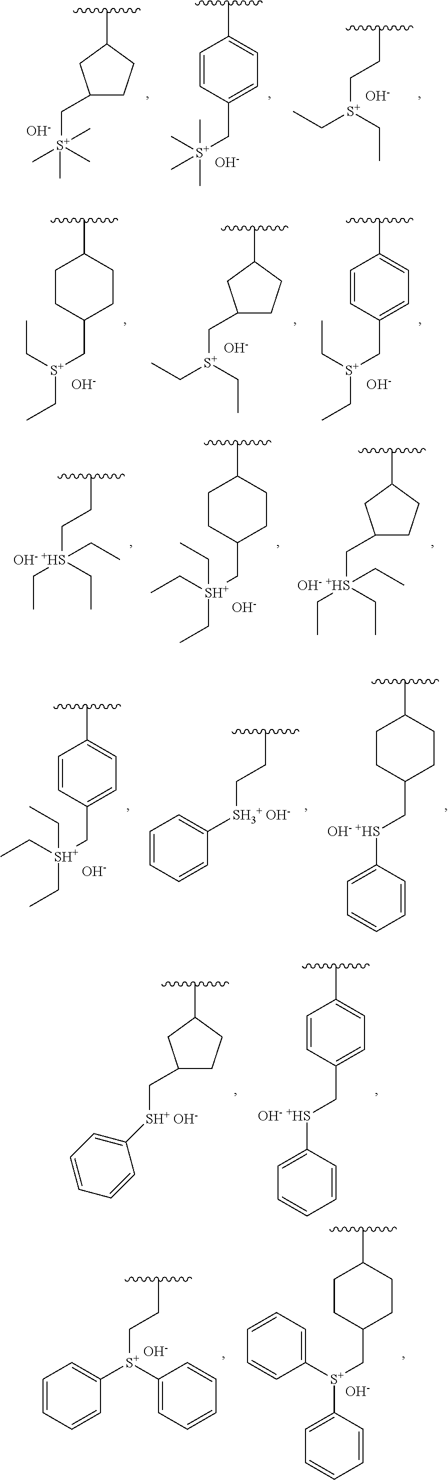

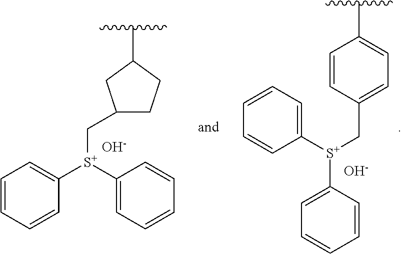

In other embodiments, the basic monomers (e.g., of a polymeric catalyst) may have a side chain with a Bronsted-Lowry base that is directly connected to the polymeric backbone. In other embodiments, the basic moieties (e.g., of a solid-supported catalyst) may have a side chain with a Bronsted-Lowry base that is directly attached to the solid support. Side chains directly connect to the polymeric backbone (e.g., of a polymeric catalyst) or basic moieties (e.g., of a solid-supported catalyst) directly attached to the solid support may can include, for example,

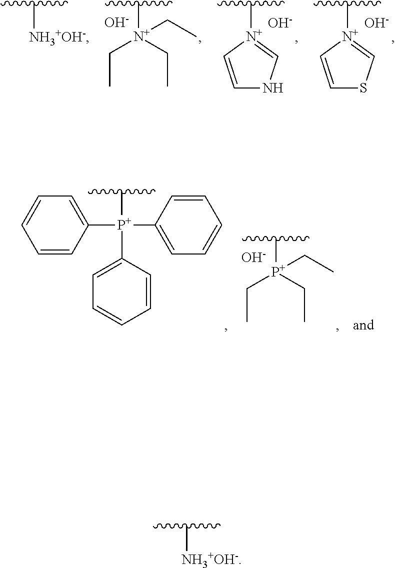

b) Ionic Monomers and Moieties

The polymeric catalysts include a plurality of ionic monomers, where as the solid-supported catalysts includes a plurality of ionic moieties attached to a solid support.

In some embodiments, a plurality of ionic monomers (e.g., of a polymeric catalyst) or a plurality of ionic moieties (e.g., of a solid-supported catalyst) has at least one anionic group. In certain embodiments, a plurality of ionic monomers (e.g., of a polymeric catalyst) or a plurality of ionic moieties (e.g., of a solid-supported catalyst) has one anionic group. In some embodiments, a plurality of ionic monomers (e.g., of a polymeric catalyst) or a plurality of ionic moieties (e.g., of a solid-supported catalyst) has two anionic, as is chemically feasible. When the ionic monomers have two or more anionic groups, the anionic groups may be the same or different.

Suitable anionic groups of the ionic monomers (e.g., of a polymeric catalyst) and the ionic moieties (e.g., of a solid-supported catalyst) may include, for example, sulfonate, phosphonate, acetate, isophthalate, and boronate. In one embodiment, each ionic monomer (e.g., of a polymeric catalyst) or each ionic moiety (e.g., of a solid-supported catalyst) includes sulfonate. In another embodiment, each ionic monomer (e.g., of a polymeric catalyst) or each ionic moiety (e.g., of a solid-supported catalyst) includes phosphonate. In yet another embodiment, the anionic group in some of the ionic monomers (e.g., of a polymeric catalyst) or the ionic moieties (e.g., of a solid-supported catalyst) is sulfonate, while the anionic group in other ionic monomers (e.g., of a polymeric catalyst) or ionic moieties (e.g., of a solid-supported catalyst) is phosphonate.

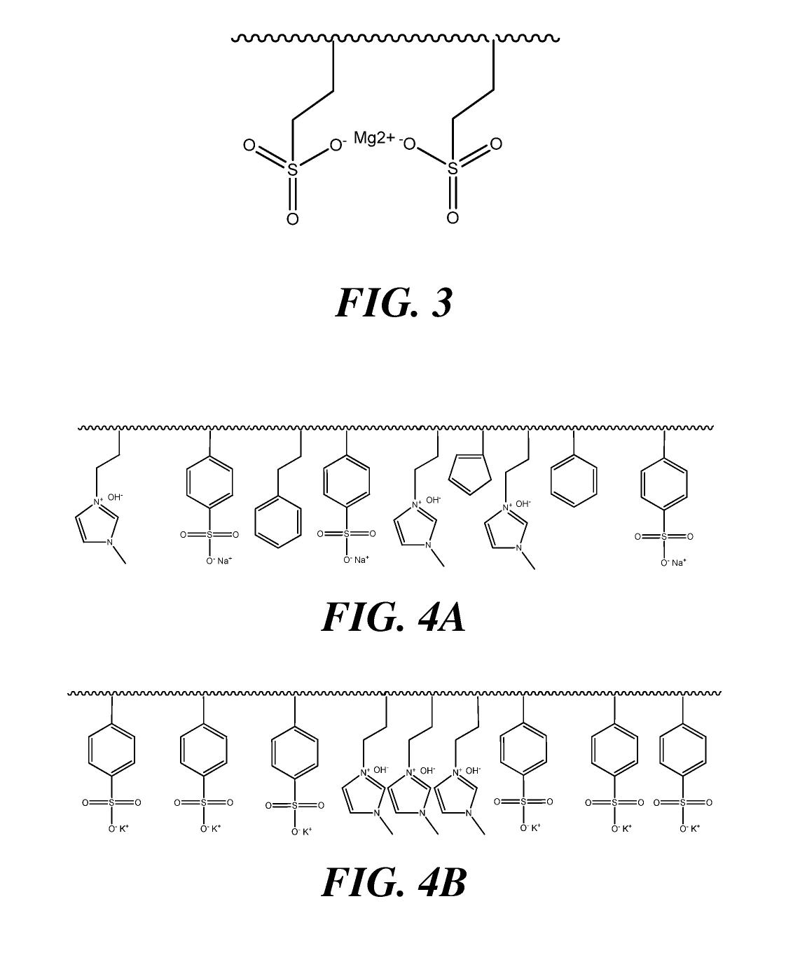

One or more counterions coordinate with one or more of the anionic groups, as is chemically feasible. Suitable counterions may include, for example, sodium, potassium, magnesium, calcium, lead, and ammonium. In some embodiments, one counterion coordinates with one anionic group. In other embodiments, one counterion may also coordinate with two or more anionic group, depending on the charge of the counterion. With reference to FIG. 3, magnesium can coordinate with two sulfonate groups.

In other embodiments, the anionic group may coordinate with a Bronsted-Lowry base in the catalyst. At least a portion of the Bronsted-Lowry bases and the anionic groups in the catalyst may form inter-monomer or inter-moiety (as the case may be) ionic associations. Inter-monomeric or inter-moiety (as the case may be) ionic associations result in salts forming between monomers or moieties in the catalyst, rather than with external counterions. In some exemplary embodiments, the ratio of basic monomers or moieties (as the case may be) engaged in inter-monomer or inter-moiety ionic associations to the total number of basic monomers or moieties may be at most 90% internally-coordinated, at most 80% internally-coordinated, at most 70% internally-coordinated, at most 60% internally-coordinated, at most 50% internally-coordinated, at most 40% internally-coordinated, at most 30% internally-coordinated, at most 20% internally-coordinated, at most 10% internally-coordinated, at most 5% internally-coordinated, at most 1% internally-coordinated, or less than 1% internally-coordinated. It should be understood that internally-coordinates sites are less likely to exchange with an ionic solution that is brought into contact with the catalyst.

In some embodiments, one or more of the ionic monomers of a polymeric catalyst are directly connected to form the polymeric backbone, or one or more of the ionic moieties of a solid-supported catalyst are directly attached to the solid support. In other embodiments, one or more of the ionic monomers (e.g., of a polymeric catalyst) or one or more ionic moieties (e.g., of a solid-supported catalyst) each independently further includes a linker connecting the anionic group to the polymeric backbone or the solid support (as the case may be). In certain embodiments, some of the anionic groups are directly connected to the polymeric backbone or directly attached to the solid support (as the case may be), while other the anionic groups are connected to the polymeric backbone or attached to the solid support (as the case may be) by a linker.

In those embodiments where the anionic group is connected to the polymeric backbone or the solid support (as the case may be) by a linker, each linker is independently selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker. In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

In other embodiments, each linker in an ionic monomer (e.g., of a polymeric catalyst) or an ionic moiety (e.g., of a solid-supported catalyst) is independently selected from:

unsubstituted alkyl linker;

alkyl linker substituted 1 to 5 substituents independently selected from oxo, hydroxy, halo, amino;

unsubstituted cycloalkyl linker;

cycloalkyl linker substituted 1 to 5 substituents independently selected from oxo, hydroxy, halo, amino;

unsubstituted alkenyl linker;

alkenyl linker substituted 1 to 5 substituents independently selected from oxo, hydroxy, halo, amino;

unsubstituted aryl linker;

aryl linker substituted 1 to 5 substituents independently selected from oxo, hydroxy, halo, amino;

unsubstituted heteroaryl linker; or

heteroaryl linker substituted 1 to 5 substituents independently selected from oxo, hydroxy, halo, amino.

Further, it should be understood that some or all of the ionic monomers (e.g., of a polymeric catalyst) or one or more ionic moieties (e.g., of a solid-supported catalyst) may have the same linker, or independently have different linkers.

In some embodiments, each ionic monomer (e.g., of a polymeric catalyst) or each ionic moiety (e.g., of a solid-supported catalyst) is independently has the structure of Formulas VII-XI:

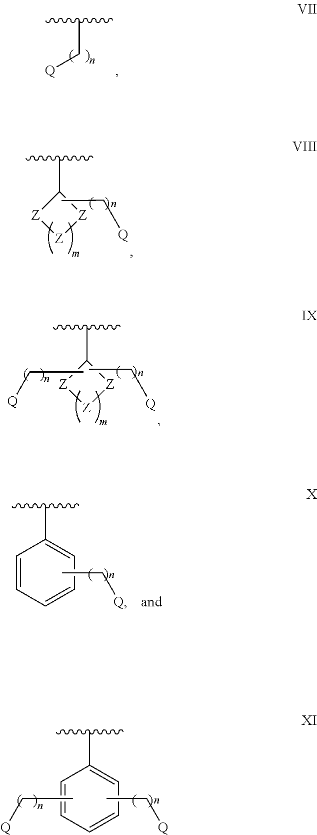

wherein:

each Q is independently SO₃⁻, PO₃⁻, BO₂⁻, C(O)O⁻, or NHR′C(O)O⁻,

R′ is alkyl, heteroalkyl, cycloalkyl, heterocycloalkyl, aryl or heteroaryl;

each Z is independently C(R²)(R³), N(R⁴), S, S(R⁵)(R⁶), S(O)(R⁵)(R⁶), SO₂, or O, wherein any two adjacent Z can (to the extent chemically feasible) be joined by a double bond, or taken together to form cycloalkyl, heterocycloalkyl, aryl or heteroaryl;

each m is independently 0, 1, 2, or 3;

each n is independently 0, 1, 2, or 3;

each R², R³ and R⁴ is independently hydrogen, alkyl, heteroalkyl, cycloalkyl, heterocyclyl, aryl, or heteroaryl; and

each R⁵ and R⁶ is independently alkyl, heteroalkyl, cycloalkyl, heterocyclyl, aryl, or heteroaryl.

In some embodiments, Z can be chosen from C(R²)(R³), N(R⁴), SO₂, and O. In some embodiments, any two adjacent Z can be taken together to form heterocycloalkyl, aryl or heteroaryl. In other embodiments, any two adjacent Z can be joined by a double bond.

In some embodiments, m is 2 or 3. In other embodiments, n is 1, 2, or 3. In some embodiments, each R², R³, and R⁴ can be independently hydrogen, alkyl, heterocyclyl, aryl, or heteroaryl. In other embodiments, each R², R³ and R⁴ can be independently heteroalkyl, cycloalkyl, heterocyclyl, or heteroaryl. In some embodiments, each R⁵ and R⁶ can be independently alkyl, heterocyclyl, aryl, or heteroaryl. In another embodiment, any two adjacent Z can be taken together to form cycloalkyl, heterocycloalkyl, aryl or heteroaryl.

In certain embodiments, the ionic monomers of the polymeric catalyst may have a side chain with an anionic group that is connected to the polymeric backbone by a linker. In certain embodiments, the ionic moieties of the solid-supported catalyst may have an anionic group that is attached to the solid support by a linker. Side chains (e.g., of a polymeric catalyst) or ionic moieties (e.g., of a solid-supported catalyst) with one or more anionic groups connected by a linker can include, for example,

wherein:

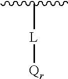

Q is as defined for Formula VII-XI;

r is an integer; and

L is an unsubstituted alkyl linker, alkyl linker substituted with oxo, unsubstituted cycloalkyl, unsubstituted aryl, unsubstituted heterocycloalkyl, or unsubstituted heteroaryl;

In other embodiments L is methyl, ethyl, propyl, or butyl. In yet other embodiments, the linker is ethanoyl, propanoyl, or benzoyl. In certain embodiments, r is 1, 2, 3, 4, or 5 (as applicable or chemically feasible).

In other embodiments, each linker is independently selected from:

unsubstituted alkyl linker;

alkyl linker substituted 1 to 5 substituents independently selected from oxo, hydroxy, halo, amino;

unsubstituted cycloalkyl linker;

cycloalkyl linker substituted 1 to 5 substituents independently selected from oxo, hydroxy, halo, amino;

unsubstituted alkenyl linker;

alkenyl linker substituted 1 to 5 substituents independently selected from oxo, hydroxy, halo, amino;

unsubstituted aryl linker;

aryl linker substituted 1 to 5 substituents independently selected from oxo, hydroxy, halo, amino;

unsubstituted heteroaryl linker; or

heteroaryl linker substituted 1 to 5 substituents independently selected from oxo, hydroxy, halo, amino.

In certain embodiments, each linker is an unsubstituted alkyl linker or an alkyl linker with an oxo substituent. In other embodiments L is methyl, ethyl, propyl, butyl. In yet other embodiments, the linker is ethanoyl, propanoyl, benzoyl. In one embodiment, each linker is —(CH₂)(CH₂)— or —(CH₂)(C═O).

In some embodiments, at least some of the ionic side chains (e.g., of a polymeric catalyst) and at least some of the ionic moieties (e.g., of a solid-supported catalyst) may be:

wherein:

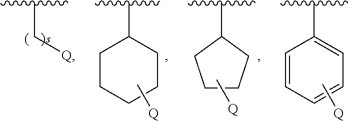

Q is as defined for Formula VII-XI; and

s is an integer.

In certain embodiments, s is 1 to 9, or 1 to 8, or 1 to 7, or 1 to 6, or 1 to 5, or 1 to 4, or 1 to 3, or 2, or 1.

In certain embodiments, at least some of the ionic side chains (e.g., of a polymeric catalyst) and at least some of the ionic moieties (e.g., of a solid-supported catalyst) may be:

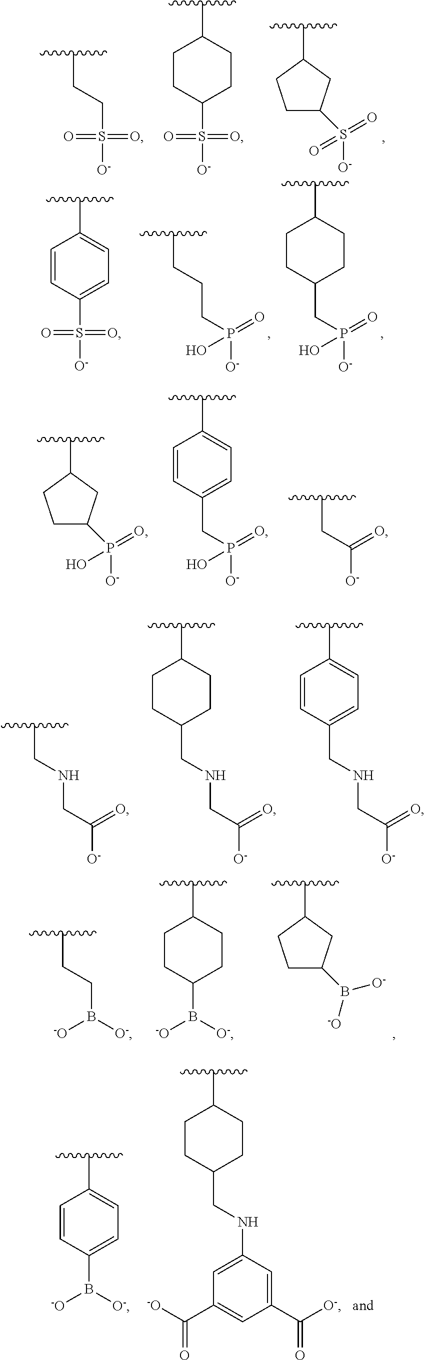

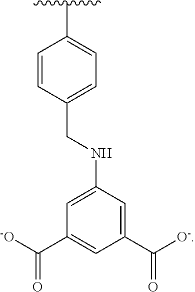

In other embodiments, the ionic monomers may have a side chain with an anionic group that is directly connected to the polymeric backbone. Side chains with an anionic group directly connected to the polymeric backbone may include, for example,

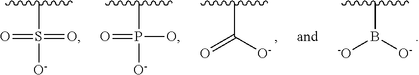

The ionic monomers (e.g., of a polymeric catalyst) or ionic moieties (e.g., of a solid-supported catalyst) can either all have the same anionic group, or can have different anionic groups. In some embodiments, each anionic group in the polymeric catalyst or solid-supported catalyst is sulfonate. In other embodiments, each anionic group in the polymeric catalyst or solid-supported catalyst is phosphonate. In yet other embodiments, the anionic group in some monomers or moieties of the polymeric catalyst or solid-supported catalyst, respectively, is sulfonate, whereas the anionic group in other monomers or moieties of the polymeric catalyst or solid-supported catalyst, respectively, is phosphonate.

c) Basic-Ionic Monomers and Moieties

Some of the monomers in the polymeric catalyst contain both the Bronsted-Lowry base and the anionic group in the same monomer. Such monomers are referred to as “basic-ionic monomers”. Similarly, some of the moieties in the solid-supported catalyst contain both the Bronsted-Lowry base and the anionic group in the same moiety. Such monomers are referred to as “basic-ionic moieties”. For example, in exemplary embodiments, the basic-ionic monomer (e.g., of a polymeric catalyst) or an basic-ionic moiety (e.g., of a solid-supported catalyst) can contain pyrrolium hydroxide and sulfonate, or phenylsulfonium hydroxide and phosphonate.

In some embodiments, the monomers (e.g., of a polymeric catalyst) or moieties (e.g., of a solid-supported catalyst) include both Bronsted-Lowry base(s) and anionic group(s), where either the Bronsted-Lowry base is connected to the polymeric backbone (e.g., of a polymeric catalyst) or attached to the solid support (e.g., of a solid-supported catalyst) by a linker, and/or the anionic group is connected to the polymeric backbone (e.g., of a polymeric catalyst) or attached to the solid support (e.g., of a solid-supported catalyst) by a linker.

It should be understood that any of the Bronsted-Lowry bases, anionic groups, and linkers (if present) suitable for the basic monomers/moieties and/or ionic monomers/moieties may be used in the basic-ionic monomers/moieties.

In certain embodiments, the Bronsted-Lowry base at each occurrence in the basic-ionic monomer (e.g., of a polymeric catalyst) or the basic-ionic moiety (e.g., of a solid-supported catalyst) is independently selected from pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.

In some embodiments, the anionic group at each occurrence in the basic-ionic monomer (e.g., of a polymeric catalyst) or the basic-ionic moiety (e.g., of a solid-supported catalyst) is independently selected from sulfonate, phosphonate, acetate, isophthalate, and boronate.

In some embodiments, the linker is unsubstituted or substituted alkyl linker, unsubstituted or substituted cycloalkyl linker, unsubstituted or substituted alkenyl linker, unsubstituted or substituted aryl linker, or unsubstituted or substituted heteroaryl linker. In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

In some embodiments, the polymeric catalyst may have at least one basic-ionic monomer with a linker connecting either the Bronsted-Lowry base or the anionic group to the polymeric backbone. In some embodiments, the solid-supported catalyst may have at least one basic-ionic moiety with a linker attaching either the Bronsted-Lowry base or the anionic group to the solid support.

In other embodiments, the monomers (e.g., of a polymeric catalyst) or moieties (e.g., of a solid-supported catalyst) can have a side chain containing both a Bronsted-Lowry base and a anionic group, where the Bronsted-Lowry base is directly connected to the polymeric backbone or attached to the solid support, the anionic group is directly connected to the polymeric backbone or attached to the solid support, or both the Bronsted-Lowry base and the anionic group are directly connected to the polymeric backbone or attached to the solid support.

Monomers that have side chains containing both a Bronsted-Lowry base and an anionic group may also be called “basic ionomers”. Basic-ionic side chains (e.g., of a polymeric catalyst) or basic-ionic moieties (e.g., of a solid-supported catalyst) that are connected by a linker can include, for example,

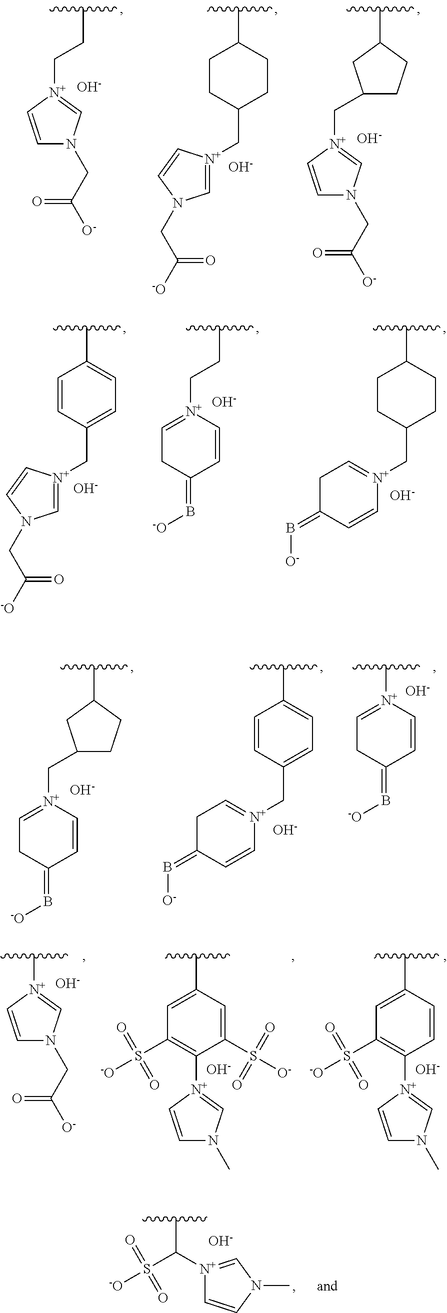

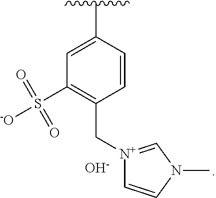

In other embodiments, the monomers may have a side chain containing both a Bronsted-Lowry base and an anionic group, where the Bronsted-Lowry base is directly connected to the polymeric backbone, the anionic group is directly connected to the polymeric backbone, or both the Bronsted-Lowry base and the anionic group are directly connected to the polymeric backbone. Such side chains in basic-ionic monomers may include, for example,

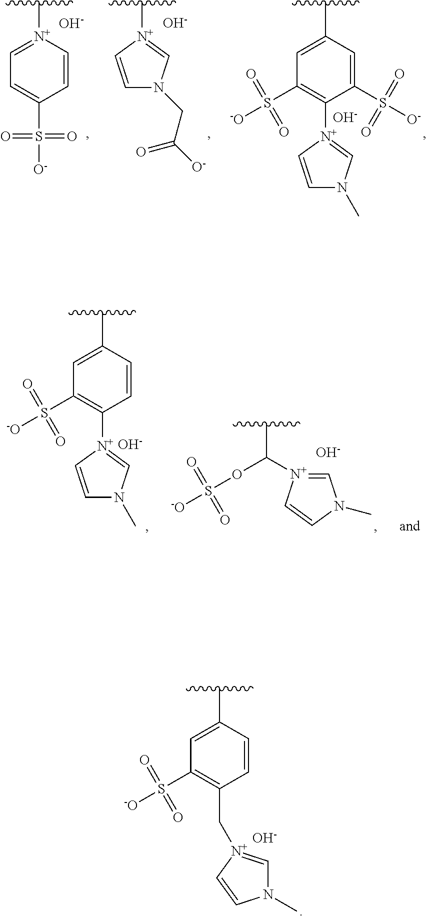

d) Hydrophobic Monomers/Moieties

In some embodiments, the polymeric catalyst further includes hydrophobic monomers connected to form the polymeric backbone. Similarly, in some embodiments, the solid-supported catalyst further includes hydrophobic moieties attached to the solid support. In either instances, each hydrophobic monomer or moiety has at least one hydrophobic group. In certain embodiments of the polymeric catalyst or solid-supported catalyst, each hydrophobic monomer or moiety, respectively, has one hydrophobic group. In certain embodiments of the polymeric catalyst or solid-supported catalyst, each hydrophobic monomer or moiety has two hydrophobic groups. In other embodiments of the polymeric catalyst or solid-supported catalyst, some of the hydrophobic monomers or moieties have one hydrophobic group, while others have two hydrophobic groups.

In some embodiments of the polymeric catalyst or solid-supported catalyst, each hydrophobic group is independently selected from an unsubstituted or substituted alkyl, an unsubstituted or substituted cycloalkyl, an unsubstituted or substituted aryl, and an unsubstituted or substituted heteroaryl. In certain embodiments of the polymeric catalyst or solid-supported catalyst, each hydrophobic group is an unsubstituted or substituted aryl, or an unsubstituted or substituted heteroaryl. In one embodiment, each hydrophobic group is phenyl. Further, it should be understood that the hydrophobic monomers may either all have the same hydrophobic group, or may have different hydrophobic groups.

In some embodiments of the polymeric catalyst, the hydrophobic group is directly connected to form the polymeric backbone. In some embodiments of the solid-supported catalyst, the hydrophobic group is directly attached to the solid support.

e) Other Characteristics of the Catalysts

In some embodiments, the basic and ionic monomers make up a substantial portion of the polymeric catalyst. In some embodiments, the basic and ionic moieties make up a substantial portion solid-supported catalyst. In certain embodiments, the basic and ionic monomers or moieties make up at least about 30%, at least about 40%, at least about 50%, at least about 60%, at least about 70%, at least about 80%, at least about 90%, at least about 95%, or at least about 99% of the monomers or moieties of the catalyst, based on the ratio of the number of basic and ionic monomers/moieties to the total number of monomers/moieties present in the catalyst.

In some embodiments, the catalyst may have between 0.01 and 20 mmol, between 0.01 and 15 mmol, between 0.01 and 12 mmol, between 0.01 and 5 mmol, between 0.01 and 4 mmol, between 0.01 and 3 mmol, between 0.01 and 2 mmol, between 0.01 and 1 mmol, between 0.05 and 10 mmol, between 1 and 8 mmol, between 2 and 7 mmol, between 3 and 6 mmol, between 1 and 5, or between 3 and 5 mmol of the total amount of Bronsted-Lowry base per gram of the catalyst.

In some embodiments, the catalyst may have between 0.01 and 20 mmol, between 0.01 and 15 mmol, between 0.01 and 12 mmol, between 0.01 and 5 mmol, between 0.01 and 4 mmol, between 0.01 and 3 mmol, between 0.01 and 2 mmol, between 0.01 and 1 mmol, between 0.05 and 10 mmol, between 1 and 8 mmol, between 2 and 7 mmol, between 3 and 6 mmol, between 1 and 5, or between 3 and 5 mmol per gram of the ionic group per gram of the catalyst. In such embodiments, the ionic group includes the anionic group listed, as well as any suitable counterion described herein (e.g., sodium, potassium, magnesium).

In some embodiments, the basic and ionic monomers make up a substantial portion of the polymeric catalyst or solid-supported catalyst. In certain embodiments, the basic and ionic monomers or moieties make up at least about 30%, at least about 40%, at least about 50%, at least about 60%, at least about 70%, at least about 80%, at least about 90%, at least about 95%, or at least about 99% of the monomers of the polymeric catalyst or solid-supported catalyst, based on the ratio of the number of basic and ionic monomers or moieties to the total number of monomers or moieties present in the polymeric catalyst or solid-supported catalyst.

The ratio of the total number of basic monomers or moieties to the total number of ionic monomers or moieties can be varied to tune the strength of the catalyst. In some embodiments, the total number of basic monomers or moieties exceeds the total number of ionic monomers or moieties in the polymer or solid support. In other embodiments, the total number of basic monomers or moieties is at least about 2, at least about 3, at least about 4, at least about 5, at least about 6, at least about 7, at least about 8, at least about 9 or at least about 10 times the total number of ionic monomers or moieties in the polymeric catalyst or solid-supported catalyst. In certain embodiments, the ratio of the total number of basic monomers or moieties to the total number of ionic monomers or moieties is about 1:1, about 2:1, about 3:1, about 4:1, about 5:1, about 6:1, about 7:1, about 8:1, about 9:1 or about 10:1.

In some embodiments, the total number of ionic monomers or moieties exceeds the total number of basic monomers or moieties in the catalyst. In other embodiments, the total number of ionic monomers or moieties is at least about 2, at least about 3, at least about 4, at least about 5, at least about 6, at least about 7, at least about 8, at least about 9 or at least about 10 times the total number of basic monomers or moieties in the polymeric catalyst or solid-supported catalyst. In certain embodiments, the ratio of the total number of ionic monomers or moieties to the total number of basic monomers or moieties is about 1:1, about 2:1, about 3:1, about 4:1, about 5:1, about 6:1, about 7:1, about 8:1, about 9:1 or about 10:1.

### Arrangement of Monomers in Polymeric Catalysts

In some embodiments, the basic monomers, the ionic monomers, the basic-ionic monomers and the hydrophobic monomers, where present, may be arranged in alternating sequence or in a random order as blocks of monomers. In some embodiments, each block has not more than twenty, fifteen, ten, six, or three monomers.

In some embodiments, the polymeric catalyst is randomly arranged in an alternating sequence. With reference to the portion of the exemplary polymeric catalyst depicted in FIG. 4A, the monomers are randomly arranged in an alternating sequence.

In other embodiments, the polymeric catalyst is randomly arranged as blocks of monomers. With reference to the portion of the exemplary polymeric catalyst depicted in FIG. 4B, the monomers are arranged in blocks of monomers.

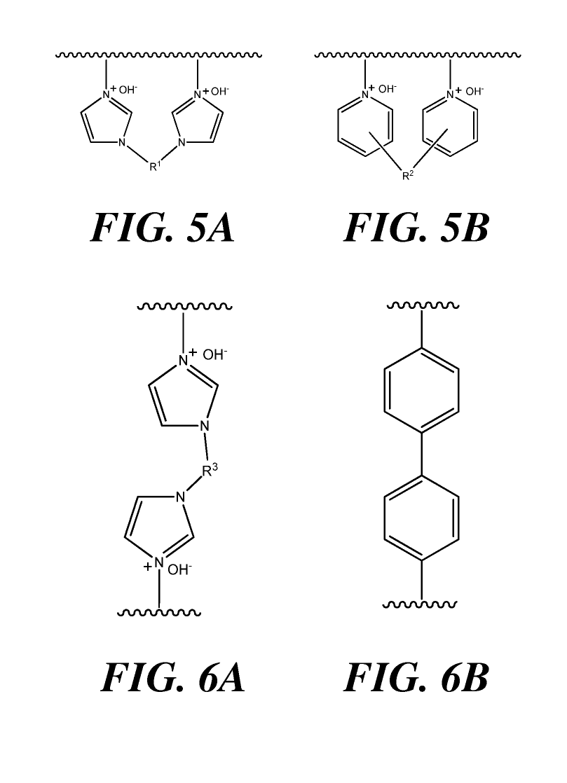

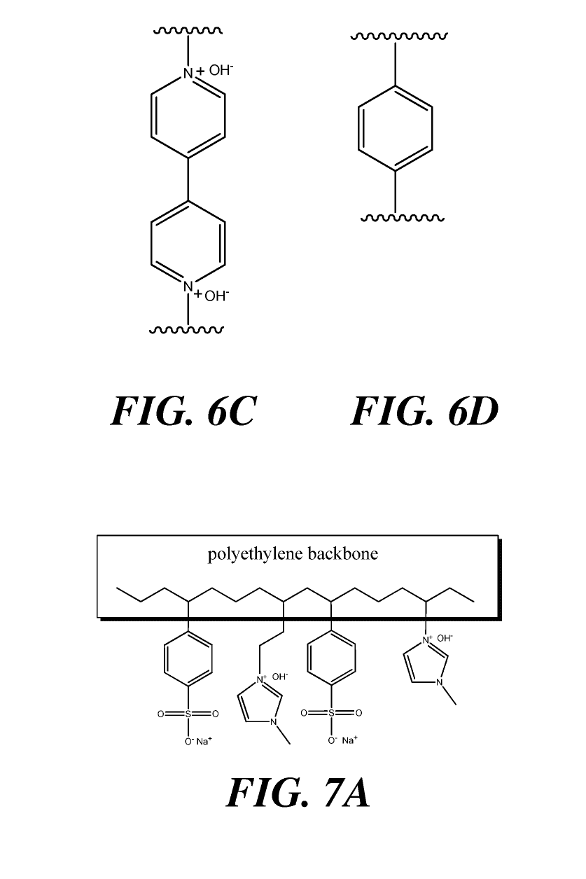

The polymeric catalyst described herein may also be cross-linked. Such cross-linked catalysts may be prepared by introducing cross-linking groups. In some embodiments, cross-linking may occur within a given polymeric chain, with reference to the portion of the exemplary catalysts depicted in FIGS. 5A and 5B. In other embodiments, cross-linking may occur between two or more polymeric chains, with reference to the portion of the exemplary catalysts in FIGS. 6A, 6B, 6C and 6D.

With reference again to FIGS. 5A, 5B and 6A, it should be understood that R¹, R² and R³, respectively, are exemplary cross linking groups. Suitable cross-linking groups that may be used to form a cross-linked polymer with the catalysts described herein include, for example, substituted or unsubstituted divinyl alkanes, substituted or unsubstituted divinyl cycloalkanes, substituted or unsubstituted divinyl aryls, substituted or unsubstituted heteroaryls, dihaloalkanes, dihaloalkenes, dihaloalkynes. For example, corss-linking groups may include divinylbenzene, diallylbenzene, dichlorobenzene, divinylmethane, dichloromethane, divinylethane, dichloroethane, divinylpropane, dichloropropane, divinylbutane, dichlorobutane, ethylene glycol, and resorcinol.

Polymeric Backbone

In some embodiments, the polymeric backbone is formed from one or more substituted or unsubstituted monomers. Polymerization processes using a wide variety of monomers are well known in the art (see, e.g., International Union of Pure and Applied Chemistry, et al., IUPAC Gold Book, Polymerization. (2000)). One such process involves monomer(s) with unsaturated substitution, such as vinyl, propenyl, butenyl, or other such substitutent(s). These types of monomers can undergo radical initiation and chain polymerization.

The polymeric backbone described herein may include, for example, polyalkylenes, polyalkenyl alcohols, polycarbonate, polyarylenes, polyaryletherketones, and polyamide-imides. In certain embodiments, the polymeric backbone may be selected from polyethylene, polypropylene, polyvinyl alcohol, polystyrene, polyurethane, polyvinyl chloride, polyphenol-aldehyde, polytetrafluoroethylene, polybutylene terephthalate, polycaprolactam, and poly(acrylonitrile butadiene styrene).

With reference to FIG. 7A, in one exemplary embodiment, the polymeric backbone is polyethylene. With reference to FIG. 7B, in another exemplary embodiment, the polymeric backbone is polyvinyl alcohol.

The polymeric backbone described herein may also include a basic group integrated as part of the polymeric backbone. Such polymeric backbones may also be called “ionomeric backbones”. In certain embodiments, the polymeric backbone may be selected from polyalkyleneammonium hydroxide, polyalkylenediammonium hydroxide, polyalkylenepyrrolium hydroxide, polyalkyleneimidazolium hydroxide, polyalkylenepyrazolium hydroxide, polyalkyleneoxazolium hydroxide, polyalkylenethiazolium hydroxide, polyalkylenepyridinium hydroxide, polyalkylenepyrimidinium hydroxide, polyalkylenepyrazinium hydroxide, polyalkylenepyradizimium hydroxide, polyalkylenethiazinium hydroxide, polyalkylenemorpholinium hydroxide, polyalkylenepiperidinium hydroxide, polyalkylenepiperizinium hydroxide, polyalkylenepyrollizinium hydroxide, polyalkylenetriphenylphosphonium hydroxide, polyalkylenetrimethylphosphonium hydroxide, polyalkylenetriethylphosphonium hydroxide, polyalkylenetripropylphosphonium hydroxide, polyalkylenetributylphosphonium hydroxide, polyalkylenetrichlorophosphonium hydroxide, polyalkylenetrifluorophosphonium hydroxide, and polyalkylenediazolium hydroxide.

With reference to FIG. 7C, in yet another exemplary embodiment, the polymeric backbone is a polyalkyleneimidazolium hydroxide.

Further, the number of atoms between side chains in the polymeric backbone may vary. In some embodiments, there are between zero and twenty atoms, zero and ten atoms, or zero and six atoms, or zero and three atoms between side chains attached to the polymeric backbone.

In some embodiments, the polymer can be a homopolymer having at least two monomer units, and where all the units contained within the polymer are derived from the same monomer in the same manner. In other embodiments, the polymer can be a heteropolymer having at least two monomer units, and where at least one monomeric unit contained within the polymer that differs from the other monomeric units in the polymer. The different monomer units in the polymer can be in a random order, in an alternating sequence of any length of a given monomer, or in blocks of monomers.

For the polymers as described herein, multiple naming conventions are well recognized in the art. For instance, a polyethylene backbone with a direct bond to an unsubstituted phenyl group (—CH₂—CH(phenyl)-CH₂—CH(phenyl)-) is also known as polystyrene. Should that phenyl group be substituted with an ethenyl group, the polymer can be named a polydivinylbenzene (—CH₂—CH(4-vinylphenyl)-CH₂—CH(4-vinylphenyl)-). Further examples of heteropolymers may include those that are functionalized after polymerization.

One suitable example would be polystyrene-co-divinylbenzene: (—CH₂—CH(phenyl)-CH₂—CH(4-ethylenephenyl)-CH₂—CH(phenyl)-CH₂—CH(4-ethylenephenyl)-). Here, the ethenyl functionality could be at the 2, 3, or 4 position on the phenyl ring.

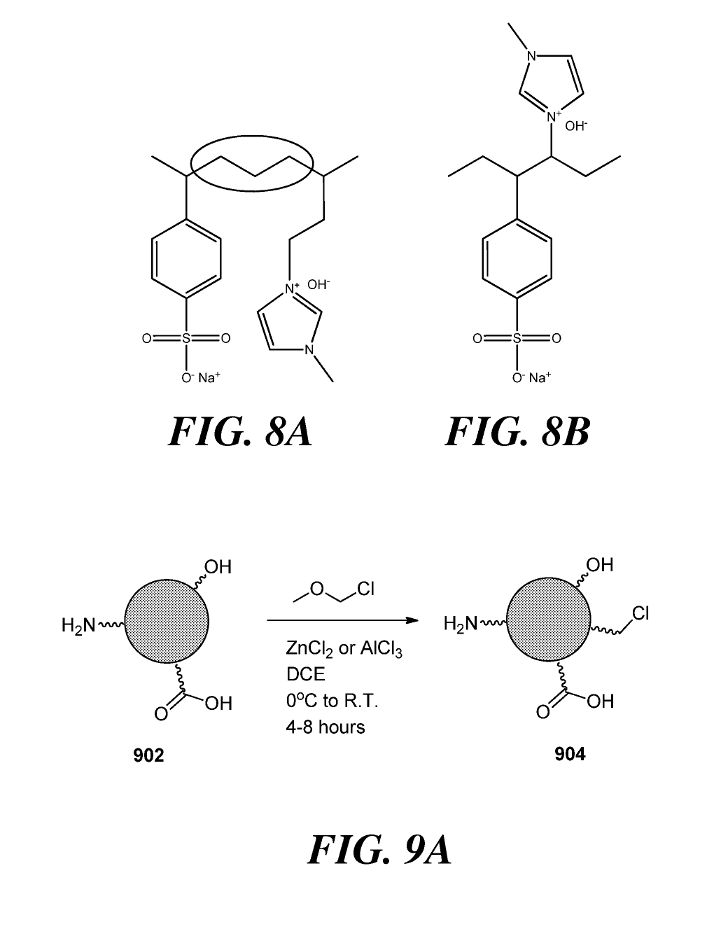

With reference to FIG. 8A, in one exemplary embodiment, there are three carbon atoms between the side chain with the Bronsted-Lowry base and the side chain with the anionic group. In another example, with reference to FIG. 8B, there are zero atoms between the side chain with the basic moiety and the side chain with the ionic moiety.

### Solid Particles for Polymeric Catalysts

The polymeric catalysts described herein can form solid particles. One of skill in the art would recognize the various known techniques and methods to make solid particles from the polymers described herein. For example, a solid particle can be formed through the procedures of emulsion or dispersion polymerization, which are known to one of skill in the art. In other embodiments, the solid particles can be formed by grinding or breaking the polymer into particles, which are also techniques and methods that are known to one of skill in the art. Methods known in the art to prepare solid particles include coating the polymers described herein on the surface of a solid core. Suitable materials for the solid core can include an inert material (e.g., aluminum oxide, corn cob, crushed glass, chipped plastic, pumice, silicon carbide, or walnut shell) or a magnetic material. Polymeric coated core particles can be made by dispersion polymerization to grow a cross-linked polymer shell around the core material, or by spray coating or melting.

Other methods known in the art to prepare solid particles include coating the polymers described herein on the surface of a solid core. The solid core can be a non-catalytic support. Suitable materials for the solid core can include an inert material (e.g., aluminum oxide, corn cob, crushed glass, chipped plastic, pumice, silicon carbide, or walnut shell) or a magnetic material. In one embodiment of the polymeric catalyst, the solid core is made up of iron. Polymeric coated core particles can be made by techniques and methods that are known to one of skill in the art, for example, by dispersion polymerization to grow a cross-linked polymer shell around the core material, or by spray coating or melting.

The solid supported polymer catalyst particle can have a solid core where the polymer is coated on the surface of the solid core. In some embodiments, at least about 5%, at least about 10%, at least about 20%, at least about 30%, at least about 40%, or at least about 50% of the catalytic activity of the solid particle can be present on or near the exterior surface of the solid particle. In some embodiments, the solid core can have an inert material or a magnetic material. In one embodiment, the solid core is made up of iron.

The solid particles coated with the polymer described herein have one or more catalytic properties. In some embodiments, at least about 50%, at least about 60%, at least about 70%, at least about 80% or at least about 90% of the catalytic activity of the solid particle is present on or near the exterior surface of the solid particle.

In some embodiments, the solid particle is substantially free of pores, for example, having no more than about 50%, no more than about 40%, no more than about 30%, no more than about 20%, no more than about 15%, no more than about 10%, no more than about 5%, or no more than about 1% of pores. Porosity can be measured by methods well known in the art, such as determining the Brunauer-Emmett-Teller (BET) surface area using the absorption of nitrogen gas on the internal and external surfaces of a material (Brunauer, S. et al., J. Am. Chem. Soc. 1938, 60:309). Other methods include measuring solvent retention by exposing the material to a suitable solvent (such as water), then removing it thermally to measure the volume of interior pores. Other solvents suitable for porosity measurement of the polymeric catalysts include, for example, polar solvents such as DMF, DMSO, acetone, and alcohols.

In other embodiments, the solid particles include a microporous gel resin. In yet other embodiments, the solid particles include a macroporous gel resin.

In other embodiments, the solid particle having the polymer coating has at least one catalytic property selected from:

a) disruption of at least one hydrogen bond in cellulosic materials;

b) intercalation of the polymer into crystalline domains of cellulosic materials;

c) cleavage of at least one glycosidic bond in cellulosic materials; and

d) disruption of at least ether linkage of lignin.

### Support of the Solid-Supported Catalysts

In certain embodiments of the solid-supported catalyst, the support may be selected from biochar, carbon, amorphous carbon, activated carbon, silica, silica gel, alumina, magnesia, titania, zirconia, clays (e.g., kaolinite), magnesium silicate, silicon carbide, zeolites (e.g., mordenite), ceramics, and any combinations thereof. In one embodiment, the support is carbon. The support for carbon support can be biochar, amorphous carbon, or activated carbon. In one embodiment, the support is activated carbon.

The carbon support can have a surface area from 0.01 to 50 m²/g of dry material. The carbon support can have a density from 0.5 to 2.5 kg/L. The support can be characterized using any suitable instrumental analysis methods or techniques known in the art, including for example scanning electron microscopy (SEM), powder X-ray diffraction (XRD), Raman spectroscopy, and Fourier Transform infrared spectroscopy (FTIR). The carbon support can be prepared from carbonaceous materials, including for example, shrimp shell, chitin, coconut shell, wood pulp, paper pulp, cotton, cellulose, hard wood, soft wood, wheat straw, sugarcane bagasse, cassava stem, corn stover, oil palm residue, bitumen, asphaltum, tar, coal, pitch, and any combinations thereof. One of skill in the art would recognize suitable methods to prepare the carbon supports used herein. See e.g., M. Inagaki, L. R. Radovic, Carbon, vol. 40, p. 2263 (2002), or A. G. Pandolfo and A. F. Hollenkamp, “Review: Carbon Properties and their role in supercapacitors,” Journal of Power Sources, vol. 157, pp. 11-27 (2006).

In other embodiments, the support is silica, silica gel, alumina, or silica-alumina. One of skill in the art would recognize suitable methods to prepare these silica- or alumina-based solid supports used herein. See e.g., Catalyst supports and supported catalysts, by A. B. Stiles, Butterworth Publishers, Stoneham Mass., 1987.

In yet other embodiments, the support is a combination of a carbon support, with one or more other supports selected from silica, silica gel, alumina, magnesia, titania, zirconia, clays (e.g., kaolinite), magnesium silicate, silicon carbide, zeolites (e.g., mordenite), and ceramics.

### Representative Examples of Catalysts

It should be understood that the polymeric catalysts and the solid-supported catalysts can include any of the Bronsted-Lowry bases, anionic groups, counterions, linkers, hydrophobic groups, cross-linking groups, and polymeric backbones or solid supports (as the case may be) described herein, as if each and every combination were listed separately. For example, in one embodiment, the catalyst can include pyrrolium hydroxide with a phenyl linker connected to a polystyrene backbone or attached to the solid support, and sulfonate connected directly to the polystyrene backbone or attached directly to the solid support (as the case may be). In another embodiment, the catalyst can include imidazolium hydroxide and phosphonate in the same monomer unit or moiety with a phenyl linker connected to a polystyrene backbone or the solid support (as the case may be).

In some embodiments, the polymeric catalyst is selected from:
poly[styrene-co-4-vinylbenzene-sodium sulfonate-co-3-methyl-1-(4-vinylbenzyl)-3H-imidazol-1-ium hydroxide-co-divinylbenzene];
poly[styrene-co-4-vinylbenzene-sodium sulfonate-co-3-ethyl-1-(4-vinylbenzyl)-3H-imidazol-1-ium hydroxide-co-divinylbenzene];
poly[styrene-co-4-vinylbenzene-sodium sulfonate-co-1-(4-vinylbenzyl)-3H-imidazol-1-ium hydroxide-co-divinylbenzene];
poly[styrene-co-4-vinylbenzene-sodium sulfonate-co-3-methyl-1-(4-vinylbenzyl)-3H-benzoimidazol-1-ium hydroxide-co-divinylbenzene];
poly[styrene-co-4-vinylbenzene-sodium sulfonate-co-1-(4-vinylbenzyl)-pyridinium-hydroxide-co-divinylbenzene];
poly[styrene-co-4-vinylbenzene-sodium sulfonate-co-1-(4-vinylbenzyl)-pyridinium-hydroxide-co-3-methyl-1-(4-vinylbenzyl)-3H-imidazol-1-ium hydroxide-co-divinylbenzene];
poly[styrene-co-4-vinylbenzene-sodium sulfonate-co-4-methyl-4-(4-vinylbenzyl)-morpholin-4-ium hydroxide-co-divinylbenzene];
poly[styrene-co-4-vinylbenzene-sodium sulfonate-co-triphenyl-(4-vinylbenzyl)-phosphonium hydroxide-co-divinylbenzene];
poly[styrene-co-4-vinylbenzene-sodium sulfonate-co-1-methyl-1-(4-vinylbenzyl)-piperdin-1-ium hydroxide-co-divinylbenzene]; and
poly[styrene-co-4-vinylbenzene-sodium sulfonate-co-triethyl-(4-vinylbenzyl)-ammonium hydroxide-co-divinylbenzene].

In some embodiments, the solid-supported catalyst is selected from:

amorphous carbon-supported pyrrolium hydroxide sodium sulfonate;

amorphous carbon-supported imidazolium hydroxide sodium sulfonate;

amorphous carbon-supported pyrazolium hydroxide sodium sulfonate;

amorphous carbon-supported oxazolium hydroxide sodium sulfonate;

amorphous carbon-supported thiazolium hydroxide sodium sulfonate;

amorphous carbon-supported pyridinium hydroxide sodium sulfonate;

amorphous carbon-supported pyrimidinium hydroxide sodium sulfonate;

amorphous carbon-supported pyrazinium hydroxide sodium sulfonate;

amorphous carbon-supported pyradizimium hydroxide sodium sulfonate;

amorphous carbon-supported thiazinium hydroxide sodium sulfonate;

amorphous carbon-supported morpholinium hydroxide sodium sulfonate;

amorphous carbon-supported piperidinium hydroxide sodium sulfonate;

amorphous carbon-supported piperizinium hydroxide sodium sulfonate;
amorphous carbon-supported pyrollizinium hydroxide sodium sulfonate;

amorphous carbon-supported triphenyl phosphonium hydroxide sodium sulfonate;

amorphous carbon-supported trimethyl phosphonium hydroxide sodium sulfonate;

amorphous carbon-supported triethyl phosphonium hydroxide sodium sulfonate;

amorphous carbon-supported tripropyl phosphonium hydroxide sodium sulfonate;

amorphous carbon-supported tributyl phosphonium hydroxide sodium sulfonate;

amorphous carbon-supported trifluoro phosphonium hydroxide sodium sulfonate;

amorphous carbon-supported sulfonium hydroxide sodium sulfonate;

amorphous carbon-supported methylsulfonium hydroxide sodium sulfonate;

amorphous carbon-supported dimethylsulfonium hydroxide sodium sulfonate;

amorphous carbon-supported trimethylsulfonium hydroxide sodium sulfonate;

amorphous carbon-supported tetramethylsulfonium hydroxide sodium sulfonate;

amorphous carbon-supported ethylsulfonium hydroxide sodium sulfonate;

amorphous carbon-supported diethylsulfonium hydroxide sodium sulfonate;

amorphous carbon-supported triethylsulfonium hydroxide sodium sulfonate;

amorphous carbon-supported tetraethylsulfonium hydroxide sodium sulfonate;

amorphous carbon-supported propylsulfonium hydroxide sodium sulfonate;

amorphous carbon-supported dipropylsulfonium hydroxide sodium sulfonate;

amorphous carbon-supported tripropylsulfonium hydroxide sodium sulfonate;

amorphous carbon-supported tetrapropylsulfonium hydroxide sodium sulfonate;

amorphous carbon-supported phenylsulfonium hydroxide sodium sulfonate;

amorphous carbon-supported diphenylsulfonium hydroxide sodium sulfonate;

amorphous carbon-supported triphenylsulfonium hydroxide sodium sulfonate;

amorphous carbon-supported tetraphenylsulfonium hydroxide sodium sulfonate;

amorphous carbon-supported pyrrolium hydroxide potassium sulfonate;

amorphous carbon-supported imidazolium hydroxide potassium sulfonate;

amorphous carbon-supported pyrazolium hydroxide potassium sulfonate;

amorphous carbon-supported oxazolium hydroxide potassium sulfonate;

amorphous carbon-supported thiazolium hydroxide potassium sulfonate;

amorphous carbon-supported pyridinium hydroxide potassium sulfonate;

amorphous carbon-supported pyrimidinium hydroxide potassium sulfonate;

amorphous carbon-supported pyrazinium hydroxide potassium sulfonate;

amorphous carbon-supported pyradizimium hydroxide potassium sulfonate;

amorphous carbon-supported thiazinium hydroxide potassium sulfonate;

amorphous carbon-supported morpholinium hydroxide potassium sulfonate;

amorphous carbon-supported piperidinium hydroxide potassium sulfonate;

amorphous carbon-supported piperizinium hydroxide potassium sulfonate;

amorphous carbon-supported pyrollizinium hydroxide potassium sulfonate;

amorphous carbon-supported triphenyl phosphonium hydroxide potassium sulfonate;

amorphous carbon-supported trimethyl phosphonium hydroxide potassium sulfonate;

amorphous carbon-supported triethyl phosphonium hydroxide potassium sulfonate;

amorphous carbon-supported tripropyl phosphonium hydroxide potassium sulfonate;

amorphous carbon-supported tributyl phosphonium hydroxide potassium sulfonate;

amorphous carbon-supported trifluoro phosphonium hydroxide potassium sulfonate;

amorphous carbon-supported sulfonium hydroxide potassium sulfonate;

amorphous carbon-supported methylsulfonium hydroxide potassium sulfonate;

amorphous carbon-supported dimethylsulfonium hydroxide potassium sulfonate;

amorphous carbon-supported trimethylsulfonium hydroxide potassium sulfonate;

amorphous carbon-supported tetramethylsulfonium hydroxide potassium sulfonate;

amorphous carbon-supported ethylsulfonium hydroxide potassium sulfonate;

amorphous carbon-supported diethylsulfonium hydroxide potassium sulfonate;

amorphous carbon-supported triethylsulfonium hydroxide potassium sulfonate;

amorphous carbon-supported tetraethylsulfonium hydroxide potassium sulfonate;

amorphous carbon-supported propylsulfonium hydroxide potassium sulfonate;

amorphous carbon-supported dipropylsulfonium hydroxide potassium sulfonate;

amorphous carbon-supported tripropylsulfonium hydroxide potassium sulfonate;

amorphous carbon-supported tetrapropylsulfonium hydroxide potassium sulfonate;

amorphous carbon-supported phenylsulfonium hydroxide potassium sulfonate;

amorphous carbon-supported diphenylsulfonium hydroxide potassium sulfonate;

amorphous carbon-supported triphenylsulfonium hydroxide potassium sulfonate;

amorphous carbon-supported tetraphenylsulfonium hydroxide potassium sulfonate;

amorphous carbon-supported pyrrolium hydroxide magnesium sulfonate;

amorphous carbon-supported imidazolium hydroxide magnesium sulfonate;

amorphous carbon-supported pyrazolium hydroxide magnesium sulfonate;

amorphous carbon-supported oxazolium hydroxide magnesium sulfonate;

amorphous carbon-supported thiazolium hydroxide magnesium sulfonate;

amorphous carbon-supported pyridinium hydroxide magnesium sulfonate;

amorphous carbon-supported pyrimidinium hydroxide magnesium sulfonate;

amorphous carbon-supported pyrazinium hydroxide magnesium sulfonate;

amorphous carbon-supported pyradizimium hydroxide magnesium sulfonate;

amorphous carbon-supported thiazinium hydroxide magnesium sulfonate;

amorphous carbon-supported morpholinium hydroxide magnesium sulfonate;

amorphous carbon-supported piperidinium hydroxide magnesium sulfonate;

amorphous carbon-supported piperizinium hydroxide magnesium sulfonate;

amorphous carbon-supported pyrollizinium hydroxide magnesium sulfonate;

amorphous carbon-supported triphenyl phosphonium hydroxide magnesium sulfonate;

amorphous carbon-supported trimethyl phosphonium hydroxide magnesium sulfonate;

amorphous carbon-supported triethyl phosphonium hydroxide magnesium sulfonate;

amorphous carbon-supported tripropyl phosphonium hydroxide magnesium sulfonate;

amorphous carbon-supported tributyl phosphonium hydroxide magnesium sulfonate;

amorphous carbon-supported trifluoro phosphonium hydroxide magnesium sulfonate;

amorphous carbon-supported sulfonium hydroxide magnesium sulfonate;

amorphous carbon-supported methylsulfonium hydroxide magnesium sulfonate;

amorphous carbon-supported dimethylsulfonium hydroxide magnesium sulfonate;

amorphous carbon-supported trimethylsulfonium hydroxide magnesium sulfonate;

amorphous carbon-supported tetramethylsulfonium hydroxide magnesium sulfonate;

amorphous carbon-supported ethylsulfonium hydroxide magnesium sulfonate;

amorphous carbon-supported diethylsulfonium hydroxide magnesium sulfonate;

amorphous carbon-supported triethylsulfonium hydroxide magnesium sulfonate;

amorphous carbon-supported tetraethylsulfonium hydroxide magnesium sulfonate;

amorphous carbon-supported propylsulfonium hydroxide magnesium sulfonate;

amorphous carbon-supported dipropylsulfonium hydroxide magnesium sulfonate;

amorphous carbon-supported tripropylsulfonium hydroxide magnesium sulfonate;

amorphous carbon-supported tetrapropylsulfonium hydroxide magnesium sulfonate;

amorphous carbon-supported phenylsulfonium hydroxide magnesium sulfonate;

amorphous carbon-supported diphenylsulfonium hydroxide magnesium sulfonate;

amorphous carbon-supported triphenylsulfonium hydroxide magnesium sulfonate;

amorphous carbon-supported tetraphenylsulfonium hydroxide magnesium sulfonate;

amorphous carbon-supported pyrrolium hydroxide calcium sulfonate;

amorphous carbon-supported imidazolium hydroxide calcium sulfonate;

amorphous carbon-supported pyrazolium hydroxide calcium sulfonate;

amorphous carbon-supported oxazolium hydroxide calcium sulfonate;

amorphous carbon-supported thiazolium hydroxide calcium sulfonate;

amorphous carbon-supported pyridinium hydroxide calcium sulfonate;

amorphous carbon-supported pyrimidinium hydroxide calcium sulfonate;

amorphous carbon-supported pyrazinium hydroxide calcium sulfonate;

amorphous carbon-supported pyradizimium hydroxide calcium sulfonate;

amorphous carbon-supported thiazinium hydroxide calcium sulfonate;

amorphous carbon-supported morpholinium hydroxide calcium sulfonate;

amorphous carbon-supported piperidinium hydroxide calcium sulfonate;

amorphous carbon-supported piperizinium hydroxide calcium sulfonate;

amorphous carbon-supported pyrollizinium hydroxide calcium sulfonate;

amorphous carbon-supported triphenyl phosphonium hydroxide calcium sulfonate;

amorphous carbon-supported trimethyl phosphonium hydroxide calcium sulfonate;

amorphous carbon-supported triethyl phosphonium hydroxide calcium sulfonate;

amorphous carbon-supported tripropyl phosphonium hydroxide calcium sulfonate;

amorphous carbon-supported tributyl phosphonium hydroxide calcium sulfonate;

amorphous carbon-supported trifluoro phosphonium hydroxide calcium sulfonate;

amorphous carbon-supported sulfonium hydroxide calcium sulfonate;

amorphous carbon-supported methylsulfonium hydroxide calcium sulfonate;

amorphous carbon-supported dimethylsulfonium hydroxide calcium sulfonate;

amorphous carbon-supported trimethylsulfonium hydroxide calcium sulfonate;

amorphous carbon-supported tetramethylsulfonium hydroxide calcium sulfonate;

amorphous carbon-supported ethylsulfonium hydroxide calcium sulfonate;

amorphous carbon-supported diethylsulfonium hydroxide calcium sulfonate;

amorphous carbon-supported triethylsulfonium hydroxide calcium sulfonate;

amorphous carbon-supported tetraethylsulfonium hydroxide calcium sulfonate;

amorphous carbon-supported propylsulfonium hydroxide calcium sulfonate;

amorphous carbon-supported dipropylsulfonium hydroxide calcium sulfonate;

amorphous carbon-supported tripropylsulfonium hydroxide calcium sulfonate;

amorphous carbon-supported tetrapropylsulfonium hydroxide calcium sulfonate;

amorphous carbon-supported phenylsulfonium hydroxide calcium sulfonate;

amorphous carbon-supported diphenylsulfonium hydroxide calcium sulfonate;

amorphous carbon-supported triphenylsulfonium hydroxide calcium sulfonate;

amorphous carbon-supported tetraphenylsulfonium hydroxide calcium sulfonate;

amorphous carbon-supported pyrrolium hydroxide sodium phosphonate;

amorphous carbon-supported imidazolium hydroxide sodium phosphonate;

amorphous carbon-supported pyrazolium hydroxide sodium phosphonate;

amorphous carbon-supported oxazolium hydroxide sodium phosphonate;

amorphous carbon-supported thiazolium hydroxide sodium phosphonate;

amorphous carbon-supported pyridinium hydroxide sodium phosphonate;

amorphous carbon-supported pyrimidinium hydroxide sodium phosphonate;

amorphous carbon-supported pyrazinium hydroxide sodium phosphonate;

amorphous carbon-supported pyradizimium hydroxide sodium phosphonate;

amorphous carbon-supported thiazinium hydroxide sodium phosphonate;

amorphous carbon-supported morpholinium hydroxide sodium phosphonate;

amorphous carbon-supported piperidinium hydroxide sodium phosphonate;

amorphous carbon-supported piperizinium hydroxide sodium phosphonate;

amorphous carbon-supported pyrollizinium hydroxide sodium phosphonate;

amorphous carbon-supported triphenyl phosphonium hydroxide sodium phosphonate;

amorphous carbon-supported trimethyl phosphonium hydroxide sodium phosphonate;

amorphous carbon-supported triethyl phosphonium hydroxide sodium phosphonate;

amorphous carbon-supported tripropyl phosphonium hydroxide sodium phosphonate;

amorphous carbon-supported tributyl phosphonium hydroxide sodium phosphonate;

amorphous carbon-supported trifluoro phosphonium hydroxide sodium phosphonate;

amorphous carbon-supported sulfonium hydroxide sodium phosphonate;

amorphous carbon-supported methylsulfonium hydroxide sodium phosphonate;

amorphous carbon-supported dimethylsulfonium hydroxide sodium phosphonate;

amorphous carbon-supported trimethylsulfonium hydroxide sodium phosphonate;

amorphous carbon-supported tetramethylsulfonium hydroxide sodium phosphonate;

amorphous carbon-supported ethylsulfonium hydroxide sodium phosphonate;

amorphous carbon-supported diethylsulfonium hydroxide sodium phosphonate;

amorphous carbon-supported triethylsulfonium hydroxide sodium phosphonate;

amorphous carbon-supported tetraethylsulfonium hydroxide sodium phosphonate;

amorphous carbon-supported propylsulfonium hydroxide sodium phosphonate;

amorphous carbon-supported dipropylsulfonium hydroxide sodium phosphonate;

amorphous carbon-supported tripropylsulfonium hydroxide sodium phosphonate;

amorphous carbon-supported tetrapropylsulfonium hydroxide sodium phosphonate;

amorphous carbon-supported phenylsulfonium hydroxide sodium phosphonate;

amorphous carbon-supported diphenylsulfonium hydroxide sodium phosphonate;

amorphous carbon-supported triphenylsulfonium hydroxide sodium phosphonate;

amorphous carbon-supported tetraphenylsulfonium hydroxide sodium phosphonate;

amorphous carbon-supported pyrrolium hydroxide potassium phosphonate;

amorphous carbon-supported imidazolium hydroxide potassium phosphonate;

amorphous carbon-supported pyrazolium hydroxide potassium phosphonate;

amorphous carbon-supported oxazolium hydroxide potassium phosphonate;

amorphous carbon-supported thiazolium hydroxide potassium phosphonate;

amorphous carbon-supported pyridinium hydroxide potassium phosphonate;

amorphous carbon-supported pyrimidinium hydroxide potassium phosphonate;

amorphous carbon-supported pyrazinium hydroxide potassium phosphonate;

amorphous carbon-supported pyradizimium hydroxide potassium phosphonate;

amorphous carbon-supported thiazinium hydroxide potassium phosphonate;

amorphous carbon-supported morpholinium hydroxide potassium phosphonate;

amorphous carbon-supported piperidinium hydroxide potassium phosphonate;

amorphous carbon-supported piperizinium hydroxide potassium phosphonate;

amorphous carbon-supported pyrollizinium hydroxide potassium phosphonate;

amorphous carbon-supported triphenyl phosphonium hydroxide potassium phosphonate;

amorphous carbon-supported trimethyl phosphonium hydroxide potassium phosphonate;

amorphous carbon-supported triethyl phosphonium hydroxide potassium phosphonate;

amorphous carbon-supported tripropyl phosphonium hydroxide potassium phosphonate;

amorphous carbon-supported tributyl phosphonium hydroxide potassium phosphonate;

amorphous carbon-supported trifluoro phosphonium hydroxide potassium phosphonate;

amorphous carbon-supported sulfonium hydroxide potassium phosphonate;

amorphous carbon-supported methylsulfonium hydroxide potassium phosphonate;

amorphous carbon-supported dimethylsulfonium hydroxide potassium phosphonate;

amorphous carbon-supported trimethylsulfonium hydroxide potassium phosphonate;

amorphous carbon-supported tetramethylsulfonium hydroxide potassium phosphonate;

amorphous carbon-supported ethylsulfonium hydroxide potassium phosphonate;

amorphous carbon-supported diethylsulfonium hydroxide potassium phosphonate;

amorphous carbon-supported triethylsulfonium hydroxide potassium phosphonate;

amorphous carbon-supported tetraethylsulfonium hydroxide potassium phosphonate;

amorphous carbon-supported propylsulfonium hydroxide potassium phosphonate;

amorphous carbon-supported dipropylsulfonium hydroxide potassium phosphonate;

amorphous carbon-supported tripropylsulfonium hydroxide potassium phosphonate;

amorphous carbon-supported tetrapropylsulfonium hydroxide potassium phosphonate;

amorphous carbon-supported phenylsulfonium hydroxide potassium phosphonate;

amorphous carbon-supported diphenylsulfonium hydroxide potassium phosphonate;

amorphous carbon-supported triphenylsulfonium hydroxide potassium phosphonate;

amorphous carbon-supported tetraphenylsulfonium hydroxide potassium phosphonate;

amorphous carbon-supported pyrrolium hydroxide magnesium phosphonate;

amorphous carbon-supported imidazolium hydroxide magnesium phosphonate;

amorphous carbon-supported pyrazolium hydroxide magnesium phosphonate;

amorphous carbon-supported oxazolium hydroxide magnesium phosphonate;

amorphous carbon-supported thiazolium hydroxide magnesium phosphonate;

amorphous carbon-supported pyridinium hydroxide magnesium phosphonate;

amorphous carbon-supported pyrimidinium hydroxide magnesium phosphonate;

amorphous carbon-supported pyrazinium hydroxide magnesium phosphonate;

amorphous carbon-supported pyradizimium hydroxide magnesium phosphonate;

amorphous carbon-supported thiazinium hydroxide magnesium phosphonate;

amorphous carbon-supported morpholinium hydroxide magnesium phosphonate;

amorphous carbon-supported piperidinium hydroxide magnesium phosphonate;

amorphous carbon-supported piperizinium hydroxide magnesium phosphonate;

amorphous carbon-supported pyrollizinium hydroxide magnesium phosphonate;

amorphous carbon-supported triphenyl phosphonium hydroxide magnesium phosphonate;

amorphous carbon-supported trimethyl phosphonium hydroxide magnesium phosphonate;

amorphous carbon-supported triethyl phosphonium hydroxide magnesium phosphonate;

amorphous carbon-supported tripropyl phosphonium hydroxide magnesium phosphonate;

amorphous carbon-supported tributyl phosphonium hydroxide magnesium phosphonate;

amorphous carbon-supported trifluoro phosphonium hydroxide magnesium phosphonate;

amorphous carbon-supported sulfonium hydroxide magnesium phosphonate;

amorphous carbon-supported methylsulfonium hydroxide magnesium phosphonate;

amorphous carbon-supported dimethylsulfonium hydroxide magnesium phosphonate;

amorphous carbon-supported trimethylsulfonium hydroxide magnesium phosphonate;

amorphous carbon-supported tetramethylsulfonium hydroxide magnesium phosphonate;

amorphous carbon-supported ethylsulfonium hydroxide magnesium phosphonate;

amorphous carbon-supported diethylsulfonium hydroxide magnesium phosphonate;

amorphous carbon-supported triethylsulfonium hydroxide magnesium phosphonate;

amorphous carbon-supported tetraethylsulfonium hydroxide magnesium phosphonate;

amorphous carbon-supported propylsulfonium hydroxide magnesium phosphonate;

amorphous carbon-supported dipropylsulfonium hydroxide magnesium phosphonate;

amorphous carbon-supported tripropylsulfonium hydroxide magnesium phosphonate;

amorphous carbon-supported tetrapropylsulfonium hydroxide magnesium phosphonate;

amorphous carbon-supported phenylsulfonium hydroxide magnesium phosphonate;

amorphous carbon-supported diphenylsulfonium hydroxide magnesium phosphonate;

amorphous carbon-supported triphenylsulfonium hydroxide magnesium phosphonate;

amorphous carbon-supported tetraphenylsulfonium hydroxide magnesium phosphonate;

amorphous carbon-supported pyrrolium hydroxide calcium phosphonate;

amorphous carbon-supported imidazolium hydroxide calcium phosphonate;

amorphous carbon-supported pyrazolium hydroxide calcium phosphonate;

amorphous carbon-supported oxazolium hydroxide calcium phosphonate;

amorphous carbon-supported thiazolium hydroxide calcium phosphonate;

amorphous carbon-supported pyridinium hydroxide calcium phosphonate;

amorphous carbon-supported pyrimidinium hydroxide calcium phosphonate;

amorphous carbon-supported pyrazinium hydroxide calcium phosphonate;

amorphous carbon-supported pyradizimium hydroxide calcium phosphonate;

amorphous carbon-supported thiazinium hydroxide calcium phosphonate;

amorphous carbon-supported morpholinium hydroxide calcium phosphonate;

amorphous carbon-supported piperidinium hydroxide calcium phosphonate;

amorphous carbon-supported piperizinium hydroxide calcium phosphonate;

amorphous carbon-supported pyrollizinium hydroxide calcium phosphonate;

amorphous carbon-supported triphenyl phosphonium hydroxide calcium phosphonate;

amorphous carbon-supported trimethyl phosphonium hydroxide calcium phosphonate;

amorphous carbon-supported triethyl phosphonium hydroxide calcium phosphonate;

amorphous carbon-supported tripropyl phosphonium hydroxide calcium phosphonate;

amorphous carbon-supported tributyl phosphonium hydroxide calcium phosphonate;

amorphous carbon-supported trifluoro phosphonium hydroxide calcium phosphonate;

amorphous carbon-supported sulfonium hydroxide calcium phosphonate;

amorphous carbon-supported methylsulfonium hydroxide calcium phosphonate;

amorphous carbon-supported dimethylsulfonium hydroxide calcium phosphonate;

amorphous carbon-supported trimethylsulfonium hydroxide calcium phosphonate;

amorphous carbon-supported tetramethylsulfonium hydroxide calcium phosphonate;

amorphous carbon-supported ethylsulfonium hydroxide calcium phosphonate;

amorphous carbon-supported diethylsulfonium hydroxide calcium phosphonate;

amorphous carbon-supported triethylsulfonium hydroxide calcium phosphonate;

amorphous carbon-supported tetraethylsulfonium hydroxide calcium phosphonate;

amorphous carbon-supported propylsulfonium hydroxide calcium phosphonate;

amorphous carbon-supported dipropylsulfonium hydroxide calcium phosphonate;

amorphous carbon-supported tripropylsulfonium hydroxide calcium phosphonate;

amorphous carbon-supported tetrapropylsulfonium hydroxide calcium phosphonate;

amorphous carbon-supported phenylsulfonium hydroxide calcium phosphonate;

amorphous carbon-supported diphenylsulfonium hydroxide calcium phosphonate;

amorphous carbon-supported triphenylsulfonium hydroxide calcium phosphonate;

amorphous carbon-supported tetraphenylsulfonium hydroxide calcium phosphonate;

amorphous carbon-supported pyrrolium hydroxide sodium acetate;

amorphous carbon-supported imidazolium hydroxide sodium acetate;

amorphous carbon-supported pyrazolium hydroxide sodium acetate;

amorphous carbon-supported oxazolium hydroxide sodium acetate;

amorphous carbon-supported thiazolium hydroxide sodium acetate;

amorphous carbon-supported pyridinium hydroxide sodium acetate;

amorphous carbon-supported pyrimidinium hydroxide sodium acetate;

amorphous carbon-supported pyrazinium hydroxide sodium acetate;

amorphous carbon-supported pyradizimium hydroxide sodium acetate;

amorphous carbon-supported thiazinium hydroxide sodium acetate;

amorphous carbon-supported morpholinium hydroxide sodium acetate;

amorphous carbon-supported piperidinium hydroxide sodium acetate;

amorphous carbon-supported piperizinium hydroxide sodium acetate;

amorphous carbon-supported pyrollizinium hydroxide sodium acetate;

amorphous carbon-supported triphenyl phosphonium hydroxide sodium acetate;

amorphous carbon-supported trimethyl phosphonium hydroxide sodium acetate;

amorphous carbon-supported triethyl phosphonium hydroxide sodium acetate;

amorphous carbon-supported tripropyl phosphonium hydroxide sodium acetate;

amorphous carbon-supported tributyl phosphonium hydroxide sodium acetate;

amorphous carbon-supported trifluoro phosphonium hydroxide sodium acetate;

amorphous carbon-supported sulfonium hydroxide sodium acetate;

amorphous carbon-supported methylsulfonium hydroxide sodium acetate;

amorphous carbon-supported dimethylsulfonium hydroxide sodium acetate;

amorphous carbon-supported trimethylsulfonium hydroxide sodium acetate;

amorphous carbon-supported tetramethylsulfonium hydroxide sodium acetate;

amorphous carbon-supported ethylsulfonium hydroxide sodium acetate;

amorphous carbon-supported diethylsulfonium hydroxide sodium acetate;

amorphous carbon-supported triethylsulfonium hydroxide sodium acetate;

amorphous carbon-supported tetraethylsulfonium hydroxide sodium acetate;

amorphous carbon-supported propylsulfonium hydroxide sodium acetate;

amorphous carbon-supported dipropylsulfonium hydroxide sodium acetate;

amorphous carbon-supported tripropylsulfonium hydroxide sodium acetate;

amorphous carbon-supported tetrapropylsulfonium hydroxide sodium acetate;

amorphous carbon-supported phenylsulfonium hydroxide sodium acetate;

amorphous carbon-supported diphenylsulfonium hydroxide sodium acetate;

amorphous carbon-supported triphenylsulfonium hydroxide sodium acetate;

amorphous carbon-supported tetraphenylsulfonium hydroxide sodium acetate;

amorphous carbon-supported pyrrolium hydroxide potassium acetate;

amorphous carbon-supported imidazolium hydroxide potassium acetate;

amorphous carbon-supported pyrazolium hydroxide potassium acetate;

amorphous carbon-supported oxazolium hydroxide potassium acetate;

amorphous carbon-supported thiazolium hydroxide potassium acetate;

amorphous carbon-supported pyridinium hydroxide potassium acetate;

amorphous carbon-supported pyrimidinium hydroxide potassium acetate;

amorphous carbon-supported pyrazinium hydroxide potassium acetate;

amorphous carbon-supported pyradizimium hydroxide potassium acetate;

amorphous carbon-supported thiazinium hydroxide potassium acetate;

amorphous carbon-supported morpholinium hydroxide potassium acetate;

amorphous carbon-supported piperidinium hydroxide potassium acetate;

amorphous carbon-supported piperizinium hydroxide potassium acetate;

amorphous carbon-supported pyrollizinium hydroxide potassium acetate;

amorphous carbon-supported triphenyl phosphonium hydroxide potassium acetate;

amorphous carbon-supported trimethyl phosphonium hydroxide potassium acetate;

amorphous carbon-supported triethyl phosphonium hydroxide potassium acetate;

amorphous carbon-supported tripropyl phosphonium hydroxide potassium acetate;

amorphous carbon-supported tributyl phosphonium hydroxide potassium acetate;

amorphous carbon-supported trifluoro phosphonium hydroxide potassium acetate;

amorphous carbon-supported sulfonium hydroxide potassium acetate;

amorphous carbon-supported methylsulfonium hydroxide potassium acetate;

amorphous carbon-supported dimethylsulfonium hydroxide potassium acetate;

amorphous carbon-supported trimethylsulfonium hydroxide potassium acetate;

amorphous carbon-supported tetramethylsulfonium hydroxide potassium acetate;

amorphous carbon-supported ethylsulfonium hydroxide potassium acetate;

amorphous carbon-supported diethylsulfonium hydroxide potassium acetate;

amorphous carbon-supported triethylsulfonium hydroxide potassium acetate;

amorphous carbon-supported tetraethylsulfonium hydroxide potassium acetate;

amorphous carbon-supported propylsulfonium hydroxide potassium acetate;

amorphous carbon-supported dipropylsulfonium hydroxide potassium acetate;

amorphous carbon-supported tripropylsulfonium hydroxide potassium acetate;

amorphous carbon-supported tetrapropylsulfonium hydroxide potassium acetate;

amorphous carbon-supported phenylsulfonium hydroxide potassium acetate;

amorphous carbon-supported diphenylsulfonium hydroxide potassium acetate;

amorphous carbon-supported triphenylsulfonium hydroxide potassium acetate;

amorphous carbon-supported tetraphenylsulfonium hydroxide potassium acetate;

amorphous carbon-supported pyrrolium hydroxide magnesium acetate;

amorphous carbon-supported imidazolium hydroxide magnesium acetate;

amorphous carbon-supported pyrazolium hydroxide magnesium acetate;

amorphous carbon-supported oxazolium hydroxide magnesium acetate;

amorphous carbon-supported thiazolium hydroxide magnesium acetate;

amorphous carbon-supported pyridinium hydroxide magnesium acetate;

amorphous carbon-supported pyrimidinium hydroxide magnesium acetate;

amorphous carbon-supported pyrazinium hydroxide magnesium acetate;

amorphous carbon-supported pyradizimium hydroxide magnesium acetate;

amorphous carbon-supported thiazinium hydroxide magnesium acetate;

amorphous carbon-supported morpholinium hydroxide magnesium acetate;

amorphous carbon-supported piperidinium hydroxide magnesium acetate;

amorphous carbon-supported piperizinium hydroxide magnesium acetate;

amorphous carbon-supported pyrollizinium hydroxide magnesium acetate;

amorphous carbon-supported triphenyl phosphonium hydroxide magnesium acetate;

amorphous carbon-supported trimethyl phosphonium hydroxide magnesium acetate;

amorphous carbon-supported triethyl phosphonium hydroxide magnesium acetate;

amorphous carbon-supported tripropyl phosphonium hydroxide magnesium acetate;

amorphous carbon-supported tributyl phosphonium hydroxide magnesium acetate;

amorphous carbon-supported trifluoro phosphonium hydroxide magnesium acetate;

amorphous carbon-supported sulfonium hydroxide magnesium acetate;

amorphous carbon-supported methylsulfonium hydroxide magnesium acetate;

amorphous carbon-supported dimethylsulfonium hydroxide magnesium acetate;

amorphous carbon-supported trimethylsulfonium hydroxide magnesium acetate;

amorphous carbon-supported tetramethylsulfonium hydroxide magnesium acetate;

amorphous carbon-supported ethylsulfonium hydroxide magnesium acetate;

amorphous carbon-supported diethylsulfonium hydroxide magnesium acetate;

amorphous carbon-supported triethylsulfonium hydroxide magnesium acetate;

amorphous carbon-supported tetraethylsulfonium hydroxide magnesium acetate;

amorphous carbon-supported propylsulfonium hydroxide magnesium acetate;

amorphous carbon-supported dipropylsulfonium hydroxide magnesium acetate;

amorphous carbon-supported tripropylsulfonium hydroxide magnesium acetate;

amorphous carbon-supported tetrapropylsulfonium hydroxide magnesium acetate;

amorphous carbon-supported phenylsulfonium hydroxide magnesium acetate;

amorphous carbon-supported diphenylsulfonium hydroxide magnesium acetate;

amorphous carbon-supported triphenylsulfonium hydroxide magnesium acetate;

amorphous carbon-supported tetraphenylsulfonium hydroxide magnesium acetate;

amorphous carbon-supported pyrrolium hydroxide calcium acetate;

amorphous carbon-supported imidazolium hydroxide calcium acetate;

amorphous carbon-supported pyrazolium hydroxide calcium acetate;

amorphous carbon-supported oxazolium hydroxide calcium acetate;

amorphous carbon-supported thiazolium hydroxide calcium acetate;

amorphous carbon-supported pyridinium hydroxide calcium acetate;

amorphous carbon-supported pyrimidinium hydroxide calcium acetate;

amorphous carbon-supported pyrazinium hydroxide calcium acetate;

amorphous carbon-supported pyradizimium hydroxide calcium acetate;

amorphous carbon-supported thiazinium hydroxide calcium acetate;

amorphous carbon-supported morpholinium hydroxide calcium acetate;

amorphous carbon-supported piperidinium hydroxide calcium acetate;

amorphous carbon-supported piperizinium hydroxide calcium acetate;

amorphous carbon-supported pyrollizinium hydroxide calcium acetate;

amorphous carbon-supported triphenyl phosphonium hydroxide calcium acetate;

amorphous carbon-supported trimethyl phosphonium hydroxide calcium acetate;

amorphous carbon-supported triethyl phosphonium hydroxide calcium acetate;

amorphous carbon-supported tripropyl phosphonium hydroxide calcium acetate;

amorphous carbon-supported tributyl phosphonium hydroxide calcium acetate;

amorphous carbon-supported trifluoro phosphonium hydroxide calcium acetate;

amorphous carbon-supported sulfonium hydroxide calcium acetate;

amorphous carbon-supported methylsulfonium hydroxide calcium acetate;

amorphous carbon-supported dimethylsulfonium hydroxide calcium acetate;

amorphous carbon-supported trimethylsulfonium hydroxide calcium acetate;

amorphous carbon-supported tetramethylsulfonium hydroxide calcium acetate;

amorphous carbon-supported ethylsulfonium hydroxide calcium acetate;

amorphous carbon-supported diethylsulfonium hydroxide calcium acetate;

amorphous carbon-supported triethylsulfonium hydroxide calcium acetate;

amorphous carbon-supported tetraethylsulfonium hydroxide calcium acetate;

amorphous carbon-supported propylsulfonium hydroxide calcium acetate;

amorphous carbon-supported dipropylsulfonium hydroxide calcium acetate;

amorphous carbon-supported tripropylsulfonium hydroxide calcium acetate;

amorphous carbon-supported tetrapropylsulfonium hydroxide calcium acetate;

amorphous carbon-supported phenylsulfonium hydroxide calcium acetate;

amorphous carbon-supported diphenylsulfonium hydroxide calcium acetate;

amorphous carbon-supported triphenylsulfonium hydroxide calcium acetate;

amorphous carbon-supported tetraphenylsulfonium hydroxide calcium acetate;

amorphous carbon-supported pyrrolium hydroxide sodium isophthalate;

amorphous carbon-supported imidazolium hydroxide sodium isophthalate;

amorphous carbon-supported pyrazolium hydroxide sodium isophthalate;

amorphous carbon-supported oxazolium hydroxide sodium isophthalate;

amorphous carbon-supported thiazolium hydroxide sodium isophthalate;

amorphous carbon-supported pyridinium hydroxide sodium isophthalate;

amorphous carbon-supported pyrimidinium hydroxide sodium isophthalate;

amorphous carbon-supported pyrazinium hydroxide sodium isophthalate;

amorphous carbon-supported pyradizimium hydroxide sodium isophthalate;

amorphous carbon-supported thiazinium hydroxide sodium isophthalate;

amorphous carbon-supported morpholinium hydroxide sodium isophthalate;

amorphous carbon-supported piperidinium hydroxide sodium isophthalate;

amorphous carbon-supported piperizinium hydroxide sodium isophthalate;

amorphous carbon-supported pyrollizinium hydroxide sodium isophthalate;

amorphous carbon-supported triphenyl phosphonium hydroxide sodium isophthalate;

amorphous carbon-supported trimethyl phosphonium hydroxide sodium isophthalate;

amorphous carbon-supported triethyl phosphonium hydroxide sodium isophthalate;

amorphous carbon-supported tripropyl phosphonium hydroxide sodium isophthalate;

amorphous carbon-supported tributyl phosphonium hydroxide sodium isophthalate;

amorphous carbon-supported trifluoro phosphonium hydroxide sodium isophthalate;

amorphous carbon-supported sulfonium hydroxide sodium isophthalate;

amorphous carbon-supported methylsulfonium hydroxide sodium isophthalate;

amorphous carbon-supported dimethylsulfonium hydroxide sodium isophthalate;

amorphous carbon-supported trimethylsulfonium hydroxide sodium isophthalate;

amorphous carbon-supported tetramethylsulfonium hydroxide sodium isophthalate;

amorphous carbon-supported ethylsulfonium hydroxide sodium isophthalate;

amorphous carbon-supported diethylsulfonium hydroxide sodium isophthalate;

amorphous carbon-supported triethylsulfonium hydroxide sodium isophthalate;

amorphous carbon-supported tetraethylsulfonium hydroxide sodium isophthalate;

amorphous carbon-supported propylsulfonium hydroxide sodium isophthalate;

amorphous carbon-supported dipropylsulfonium hydroxide sodium isophthalate;

amorphous carbon-supported tripropylsulfonium hydroxide sodium isophthalate;

amorphous carbon-supported tetrapropylsulfonium hydroxide sodium isophthalate;

amorphous carbon-supported phenylsulfonium hydroxide sodium isophthalate;

amorphous carbon-supported diphenylsulfonium hydroxide sodium isophthalate;

amorphous carbon-supported triphenylsulfonium hydroxide sodium isophthalate;

amorphous carbon-supported tetraphenylsulfonium hydroxide sodium isophthalate;

amorphous carbon-supported pyrrolium hydroxide potassium isophthalate;

amorphous carbon-supported imidazolium hydroxide potassium isophthalate;

amorphous carbon-supported pyrazolium hydroxide potassium isophthalate;

amorphous carbon-supported oxazolium hydroxide potassium isophthalate;

amorphous carbon-supported thiazolium hydroxide potassium isophthalate;

amorphous carbon-supported pyridinium hydroxide potassium isophthalate;

amorphous carbon-supported pyrimidinium hydroxide potassium isophthalate;

amorphous carbon-supported pyrazinium hydroxide potassium isophthalate;

amorphous carbon-supported pyradizimium hydroxide potassium isophthalate;

amorphous carbon-supported thiazinium hydroxide potassium isophthalate;

amorphous carbon-supported morpholinium hydroxide potassium isophthalate;

amorphous carbon-supported piperidinium hydroxide potassium isophthalate;

amorphous carbon-supported piperizinium hydroxide potassium isophthalate;

amorphous carbon-supported pyrollizinium hydroxide potassium isophthalate;

amorphous carbon-supported triphenyl phosphonium hydroxide potassium isophthalate;

amorphous carbon-supported trimethyl phosphonium hydroxide potassium isophthalate;

amorphous carbon-supported triethyl phosphonium hydroxide potassium isophthalate;

amorphous carbon-supported tripropyl phosphonium hydroxide potassium isophthalate;

amorphous carbon-supported tributyl phosphonium hydroxide potassium isophthalate;

amorphous carbon-supported trifluoro phosphonium hydroxide potassium isophthalate;

amorphous carbon-supported sulfonium hydroxide potassium isophthalate;

amorphous carbon-supported methylsulfonium hydroxide potassium isophthalate;

amorphous carbon-supported dimethylsulfonium hydroxide potassium isophthalate;

amorphous carbon-supported trimethylsulfonium hydroxide potassium isophthalate;

amorphous carbon-supported tetramethylsulfonium hydroxide potassium isophthalate;

amorphous carbon-supported ethylsulfonium hydroxide potassium isophthalate;

amorphous carbon-supported diethylsulfonium hydroxide potassium isophthalate;

amorphous carbon-supported triethylsulfonium hydroxide potassium isophthalate;

amorphous carbon-supported tetraethylsulfonium hydroxide potassium isophthalate;

amorphous carbon-supported propylsulfonium hydroxide potassium isophthalate;

amorphous carbon-supported dipropylsulfonium hydroxide potassium isophthalate;

amorphous carbon-supported tripropylsulfonium hydroxide potassium isophthalate;

amorphous carbon-supported tetrapropylsulfonium hydroxide potassium isophthalate;

amorphous carbon-supported phenylsulfonium hydroxide potassium isophthalate;

amorphous carbon-supported diphenylsulfonium hydroxide potassium isophthalate;

amorphous carbon-supported triphenylsulfonium hydroxide potassium isophthalate;

amorphous carbon-supported tetraphenylsulfonium hydroxide potassium isophthalate;

amorphous carbon-supported pyrrolium hydroxide magnesium isophthalate;

amorphous carbon-supported imidazolium hydroxide magnesium isophthalate;

amorphous carbon-supported pyrazolium hydroxide magnesium isophthalate;

amorphous carbon-supported oxazolium hydroxide magnesium isophthalate;

amorphous carbon-supported thiazolium hydroxide magnesium isophthalate;

amorphous carbon-supported pyridinium hydroxide magnesium isophthalate;

amorphous carbon-supported pyrimidinium hydroxide magnesium isophthalate;

amorphous carbon-supported pyrazinium hydroxide magnesium isophthalate;

amorphous carbon-supported pyradizimium hydroxide magnesium isophthalate;

amorphous carbon-supported thiazinium hydroxide magnesium isophthalate;

amorphous carbon-supported morpholinium hydroxide magnesium isophthalate;

amorphous carbon-supported piperidinium hydroxide magnesium isophthalate;

amorphous carbon-supported piperizinium hydroxide magnesium isophthalate;

amorphous carbon-supported pyrollizinium hydroxide magnesium isophthalate;

amorphous carbon-supported triphenyl phosphonium hydroxide magnesium isophthalate;

amorphous carbon-supported trimethyl phosphonium hydroxide magnesium isophthalate;

amorphous carbon-supported triethyl phosphonium hydroxide magnesium isophthalate;

amorphous carbon-supported tripropyl phosphonium hydroxide magnesium isophthalate;

amorphous carbon-supported tributyl phosphonium hydroxide magnesium isophthalate;

amorphous carbon-supported trifluoro phosphonium hydroxide magnesium isophthalate;

amorphous carbon-supported sulfonium hydroxide magnesium isophthalate;

amorphous carbon-supported methylsulfonium hydroxide magnesium isophthalate;

amorphous carbon-supported dimethylsulfonium hydroxide magnesium isophthalate;

amorphous carbon-supported trimethylsulfonium hydroxide magnesium isophthalate;

amorphous carbon-supported tetramethylsulfonium hydroxide magnesium isophthalate;

amorphous carbon-supported ethylsulfonium hydroxide magnesium isophthalate;

amorphous carbon-supported diethylsulfonium hydroxide magnesium isophthalate;

amorphous carbon-supported triethylsulfonium hydroxide magnesium isophthalate;

amorphous carbon-supported tetraethylsulfonium hydroxide magnesium isophthalate;

amorphous carbon-supported propylsulfonium hydroxide magnesium isophthalate;

amorphous carbon-supported dipropylsulfonium hydroxide magnesium isophthalate;

amorphous carbon-supported tripropylsulfonium hydroxide magnesium isophthalate;

amorphous carbon-supported tetrapropylsulfonium hydroxide magnesium isophthalate;

amorphous carbon-supported phenylsulfonium hydroxide magnesium isophthalate;

amorphous carbon-supported diphenylsulfonium hydroxide magnesium isophthalate;

amorphous carbon-supported triphenylsulfonium hydroxide magnesium isophthalate;

amorphous carbon-supported tetraphenylsulfonium hydroxide magnesium isophthalate;

amorphous carbon-supported pyrrolium hydroxide calcium isophthalate;

amorphous carbon-supported imidazolium hydroxide calcium isophthalate;

amorphous carbon-supported pyrazolium hydroxide calcium isophthalate;

amorphous carbon-supported oxazolium hydroxide calcium isophthalate;

amorphous carbon-supported thiazolium hydroxide calcium isophthalate;

amorphous carbon-supported pyridinium hydroxide calcium isophthalate;

amorphous carbon-supported pyrimidinium hydroxide calcium isophthalate;

amorphous carbon-supported pyrazinium hydroxide calcium isophthalate;

amorphous carbon-supported pyradizimium hydroxide calcium isophthalate;

amorphous carbon-supported thiazinium hydroxide calcium isophthalate;

amorphous carbon-supported morpholinium hydroxide calcium isophthalate;

amorphous carbon-supported piperidinium hydroxide calcium isophthalate;

amorphous carbon-supported piperizinium hydroxide calcium isophthalate;

amorphous carbon-supported pyrollizinium hydroxide calcium isophthalate;

amorphous carbon-supported triphenyl phosphonium hydroxide calcium isophthalate;

amorphous carbon-supported trimethyl phosphonium hydroxide calcium isophthalate;

amorphous carbon-supported triethyl phosphonium hydroxide calcium isophthalate;

amorphous carbon-supported tripropyl phosphonium hydroxide calcium isophthalate;

amorphous carbon-supported tributyl phosphonium hydroxide calcium isophthalate;

amorphous carbon-supported trifluoro phosphonium hydroxide calcium isophthalate;

amorphous carbon-supported sulfonium hydroxide calcium isophthalate;

amorphous carbon-supported methylsulfonium hydroxide calcium isophthalate;

amorphous carbon-supported dimethylsulfonium hydroxide calcium isophthalate;

amorphous carbon-supported trimethylsulfonium hydroxide calcium isophthalate;

amorphous carbon-supported tetramethylsulfonium hydroxide calcium isophthalate;

amorphous carbon-supported ethylsulfonium hydroxide calcium isophthalate;

amorphous carbon-supported diethylsulfonium hydroxide calcium isophthalate;

amorphous carbon-supported triethylsulfonium hydroxide calcium isophthalate;

amorphous carbon-supported tetraethylsulfonium hydroxide calcium isophthalate;

amorphous carbon-supported propylsulfonium hydroxide calcium isophthalate;

amorphous carbon-supported dipropylsulfonium hydroxide calcium isophthalate;

amorphous carbon-supported tripropylsulfonium hydroxide calcium isophthalate;

amorphous carbon-supported tetrapropylsulfonium hydroxide calcium isophthalate;

amorphous carbon-supported phenylsulfonium hydroxide calcium isophthalate;

amorphous carbon-supported diphenylsulfonium hydroxide calcium isophthalate;

amorphous carbon-supported triphenylsulfonium hydroxide calcium isophthalate;

amorphous carbon-supported tetraphenylsulfonium hydroxide calcium isophthalate;

amorphous carbon-supported pyrrolium hydroxide sodium boronate;

amorphous carbon-supported imidazolium hydroxide sodium boronate;

amorphous carbon-supported pyrazolium hydroxide sodium boronate;

amorphous carbon-supported oxazolium hydroxide sodium boronate;

amorphous carbon-supported thiazolium hydroxide sodium boronate;

amorphous carbon-supported pyridinium hydroxide sodium boronate;

amorphous carbon-supported pyrimidinium hydroxide sodium boronate;

amorphous carbon-supported pyrazinium hydroxide sodium boronate;

amorphous carbon-supported pyradizimium hydroxide sodium boronate;

amorphous carbon-supported thiazinium hydroxide sodium boronate;

amorphous carbon-supported morpholinium hydroxide sodium boronate;

amorphous carbon-supported piperidinium hydroxide sodium boronate;

amorphous carbon-supported piperizinium hydroxide sodium boronate;

amorphous carbon-supported pyrollizinium hydroxide sodium boronate;

amorphous carbon-supported triphenyl phosphonium hydroxide sodium boronate;

amorphous carbon-supported trimethyl phosphonium hydroxide sodium boronate;

amorphous carbon-supported triethyl phosphonium hydroxide sodium boronate;

amorphous carbon-supported tripropyl phosphonium hydroxide sodium boronate;

amorphous carbon-supported tributyl phosphonium hydroxide sodium boronate;

amorphous carbon-supported trifluoro phosphonium hydroxide sodium boronate;

amorphous carbon-supported sulfonium hydroxide sodium boronate;

amorphous carbon-supported methylsulfonium hydroxide sodium boronate;

amorphous carbon-supported dimethylsulfonium hydroxide sodium boronate;

amorphous carbon-supported trimethylsulfonium hydroxide sodium boronate;

amorphous carbon-supported tetramethylsulfonium hydroxide sodium boronate;

amorphous carbon-supported ethylsulfonium hydroxide sodium boronate;

amorphous carbon-supported diethylsulfonium hydroxide sodium boronate;

amorphous carbon-supported triethylsulfonium hydroxide sodium boronate;

amorphous carbon-supported tetraethylsulfonium hydroxide sodium boronate;

amorphous carbon-supported propylsulfonium hydroxide sodium boronate;

amorphous carbon-supported dipropylsulfonium hydroxide sodium boronate;

amorphous carbon-supported tripropylsulfonium hydroxide sodium boronate;

amorphous carbon-supported tetrapropylsulfonium hydroxide sodium boronate;

amorphous carbon-supported phenylsulfonium hydroxide sodium boronate;

amorphous carbon-supported diphenylsulfonium hydroxide sodium boronate;

amorphous carbon-supported triphenylsulfonium hydroxide sodium boronate;

amorphous carbon-supported tetraphenylsulfonium hydroxide sodium boronate;

amorphous carbon-supported pyrrolium hydroxide potassium boronate;

amorphous carbon-supported imidazolium hydroxide potassium boronate;

amorphous carbon-supported pyrazolium hydroxide potassium boronate;

amorphous carbon-supported oxazolium hydroxide potassium boronate;

amorphous carbon-supported thiazolium hydroxide potassium boronate;

amorphous carbon-supported pyridinium hydroxide potassium boronate;

amorphous carbon-supported pyrimidinium hydroxide potassium boronate;

amorphous carbon-supported pyrazinium hydroxide potassium boronate;

amorphous carbon-supported pyradizimium hydroxide potassium boronate;

amorphous carbon-supported thiazinium hydroxide potassium boronate;

amorphous carbon-supported morpholinium hydroxide potassium boronate;

amorphous carbon-supported piperidinium hydroxide potassium boronate;

amorphous carbon-supported piperizinium hydroxide potassium boronate;

amorphous carbon-supported pyrollizinium hydroxide potassium boronate;

amorphous carbon-supported triphenyl phosphonium hydroxide potassium boronate;

amorphous carbon-supported trimethyl phosphonium hydroxide potassium boronate;

amorphous carbon-supported triethyl phosphonium hydroxide potassium boronate;

amorphous carbon-supported tripropyl phosphonium hydroxide potassium boronate;

amorphous carbon-supported tributyl phosphonium hydroxide potassium boronate;

amorphous carbon-supported trifluoro phosphonium hydroxide potassium boronate;

amorphous carbon-supported sulfonium hydroxide potassium boronate;

amorphous carbon-supported methylsulfonium hydroxide potassium boronate;

amorphous carbon-supported dimethylsulfonium hydroxide potassium boronate;

amorphous carbon-supported trimethylsulfonium hydroxide potassium boronate;

amorphous carbon-supported tetramethylsulfonium hydroxide potassium boronate;

amorphous carbon-supported ethylsulfonium hydroxide potassium boronate;

amorphous carbon-supported diethylsulfonium hydroxide potassium boronate;

amorphous carbon-supported triethylsulfonium hydroxide potassium boronate;

amorphous carbon-supported tetraethylsulfonium hydroxide potassium boronate;

amorphous carbon-supported propylsulfonium hydroxide potassium boronate;

amorphous carbon-supported dipropylsulfonium hydroxide potassium boronate;

amorphous carbon-supported tripropylsulfonium hydroxide potassium boronate;

amorphous carbon-supported tetrapropylsulfonium hydroxide potassium boronate;

amorphous carbon-supported phenylsulfonium hydroxide potassium boronate;

amorphous carbon-supported diphenylsulfonium hydroxide potassium boronate;

amorphous carbon-supported triphenylsulfonium hydroxide potassium boronate;

amorphous carbon-supported tetraphenylsulfonium hydroxide potassium boronate;

amorphous carbon-supported pyrrolium hydroxide magnesium boronate;

amorphous carbon-supported imidazolium hydroxide magnesium boronate;

amorphous carbon-supported pyrazolium hydroxide magnesium boronate;

amorphous carbon-supported oxazolium hydroxide magnesium boronate;

amorphous carbon-supported thiazolium hydroxide magnesium boronate;

amorphous carbon-supported pyridinium hydroxide magnesium boronate;

amorphous carbon-supported pyrimidinium hydroxide magnesium boronate;

amorphous carbon-supported pyrazinium hydroxide magnesium boronate;

amorphous carbon-supported pyradizimium hydroxide magnesium boronate;

amorphous carbon-supported thiazinium hydroxide magnesium boronate;

amorphous carbon-supported morpholinium hydroxide magnesium boronate;

amorphous carbon-supported piperidinium hydroxide magnesium boronate;

amorphous carbon-supported piperizinium hydroxide magnesium boronate;

amorphous carbon-supported pyrollizinium hydroxide magnesium boronate;

amorphous carbon-supported triphenyl phosphonium hydroxide magnesium boronate;

amorphous carbon-supported trimethyl phosphonium hydroxide magnesium boronate;

amorphous carbon-supported triethyl phosphonium hydroxide magnesium boronate;

amorphous carbon-supported tripropyl phosphonium hydroxide magnesium boronate;

amorphous carbon-supported tributyl phosphonium hydroxide magnesium boronate;

amorphous carbon-supported trifluoro phosphonium hydroxide magnesium boronate;

amorphous carbon-supported sulfonium hydroxide magnesium boronate;

amorphous carbon-supported methylsulfonium hydroxide magnesium boronate;

amorphous carbon-supported dimethylsulfonium hydroxide magnesium boronate;

amorphous carbon-supported trimethylsulfonium hydroxide magnesium boronate;

amorphous carbon-supported tetramethylsulfonium hydroxide magnesium boronate;

amorphous carbon-supported ethylsulfonium hydroxide magnesium boronate;

amorphous carbon-supported diethylsulfonium hydroxide magnesium boronate;

amorphous carbon-supported triethylsulfonium hydroxide magnesium boronate;

amorphous carbon-supported tetraethylsulfonium hydroxide magnesium boronate;

amorphous carbon-supported propylsulfonium hydroxide magnesium boronate;

amorphous carbon-supported dipropylsulfonium hydroxide magnesium boronate;

amorphous carbon-supported tripropylsulfonium hydroxide magnesium boronate;

amorphous carbon-supported tetrapropylsulfonium hydroxide magnesium boronate;

amorphous carbon-supported phenylsulfonium hydroxide magnesium boronate;

amorphous carbon-supported diphenylsulfonium hydroxide magnesium boronate;

amorphous carbon-supported triphenylsulfonium hydroxide magnesium boronate;

amorphous carbon-supported tetraphenylsulfonium hydroxide magnesium boronate;

amorphous carbon-supported pyrrolium hydroxide calcium boronate;

amorphous carbon-supported imidazolium hydroxide calcium boronate;

amorphous carbon-supported pyrazolium hydroxide calcium boronate;

amorphous carbon-supported oxazolium hydroxide calcium boronate;

amorphous carbon-supported thiazolium hydroxide calcium boronate;

amorphous carbon-supported pyridinium hydroxide calcium boronate;

amorphous carbon-supported pyrimidinium hydroxide calcium boronate;

amorphous carbon-supported pyrazinium hydroxide calcium boronate;

amorphous carbon-supported pyradizimium hydroxide calcium boronate;

amorphous carbon-supported thiazinium hydroxide calcium boronate;

amorphous carbon-supported morpholinium hydroxide calcium boronate;

amorphous carbon-supported piperidinium hydroxide calcium boronate;

amorphous carbon-supported piperizinium hydroxide calcium boronate;

amorphous carbon-supported pyrollizinium hydroxide calcium boronate;

amorphous carbon-supported triphenyl phosphonium hydroxide calcium boronate;

amorphous carbon-supported trimethyl phosphonium hydroxide calcium boronate;

amorphous carbon-supported triethyl phosphonium hydroxide calcium boronate;

amorphous carbon-supported tripropyl phosphonium hydroxide calcium boronate;

amorphous carbon-supported tributyl phosphonium hydroxide calcium boronate;

amorphous carbon-supported trifluoro phosphonium hydroxide calcium boronate;

amorphous carbon-supported sulfonium hydroxide calcium boronate;

amorphous carbon-supported methylsulfonium hydroxide calcium boronate;

amorphous carbon-supported dimethylsulfonium hydroxide calcium boronate;

amorphous carbon-supported trimethylsulfonium hydroxide calcium boronate;

amorphous carbon-supported tetramethylsulfonium hydroxide calcium boronate;

amorphous carbon-supported ethylsulfonium hydroxide calcium boronate;

amorphous carbon-supported diethylsulfonium hydroxide calcium boronate;

amorphous carbon-supported triethylsulfonium hydroxide calcium boronate;

amorphous carbon-supported tetraethylsulfonium hydroxide calcium boronate;

amorphous carbon-supported propylsulfonium hydroxide calcium boronate;

amorphous carbon-supported dipropylsulfonium hydroxide calcium boronate;

amorphous carbon-supported tripropylsulfonium hydroxide calcium boronate;

amorphous carbon-supported tetrapropylsulfonium hydroxide calcium boronate;

amorphous carbon-supported phenylsulfonium hydroxide calcium boronate;

amorphous carbon-supported diphenylsulfonium hydroxide calcium boronate;

amorphous carbon-supported triphenylsulfonium hydroxide calcium boronate;

amorphous carbon-supported tetraphenylsulfonium hydroxide calcium boronate;

activated carbon-supported pyrrolium hydroxide sodium sulfonate;

activated carbon-supported imidazolium hydroxide sodium sulfonate;

activated carbon-supported pyrazolium hydroxide sodium sulfonate;

activated carbon-supported oxazolium hydroxide sodium sulfonate;

activated carbon-supported thiazolium hydroxide sodium sulfonate;

activated carbon-supported pyridinium hydroxide sodium sulfonate;

activated carbon-supported pyrimidinium hydroxide sodium sulfonate;

activated carbon-supported pyrazinium hydroxide sodium sulfonate;

activated carbon-supported pyradizimium hydroxide sodium sulfonate;

activated carbon-supported thiazinium hydroxide sodium sulfonate;

activated carbon-supported morpholinium hydroxide sodium sulfonate;

activated carbon-supported piperidinium hydroxide sodium sulfonate;

activated carbon-supported piperizinium hydroxide sodium sulfonate;

activated carbon-supported pyrollizinium hydroxide sodium sulfonate;

activated carbon-supported triphenyl phosphonium hydroxide sodium sulfonate;

activated carbon-supported trimethyl phosphonium hydroxide sodium sulfonate;

activated carbon-supported triethyl phosphonium hydroxide sodium sulfonate;

activated carbon-supported tripropyl phosphonium hydroxide sodium sulfonate;

activated carbon-supported tributyl phosphonium hydroxide sodium sulfonate;

activated carbon-supported trifluoro phosphonium hydroxide sodium sulfonate;

activated carbon-supported sulfonium hydroxide sodium sulfonate;

activated carbon-supported methylsulfonium hydroxide sodium sulfonate;

activated carbon-supported dimethylsulfonium hydroxide sodium sulfonate;

activated carbon-supported trimethylsulfonium hydroxide sodium sulfonate;

activated carbon-supported tetramethylsulfonium hydroxide sodium sulfonate;

activated carbon-supported ethylsulfonium hydroxide sodium sulfonate;

activated carbon-supported diethylsulfonium hydroxide sodium sulfonate;

activated carbon-supported triethylsulfonium hydroxide sodium sulfonate;

activated carbon-supported tetraethylsulfonium hydroxide sodium sulfonate;

activated carbon-supported propylsulfonium hydroxide sodium sulfonate;

activated carbon-supported dipropylsulfonium hydroxide sodium sulfonate;

activated carbon-supported tripropylsulfonium hydroxide sodium sulfonate;

activated carbon-supported tetrapropylsulfonium hydroxide sodium sulfonate;

activated carbon-supported phenylsulfonium hydroxide sodium sulfonate;

activated carbon-supported diphenylsulfonium hydroxide sodium sulfonate;

activated carbon-supported triphenylsulfonium hydroxide sodium sulfonate;

activated carbon-supported tetraphenylsulfonium hydroxide sodium sulfonate;

activated carbon-supported pyrrolium hydroxide potassium sulfonate;

activated carbon-supported imidazolium hydroxide potassium sulfonate;

activated carbon-supported pyrazolium hydroxide potassium sulfonate;

activated carbon-supported oxazolium hydroxide potassium sulfonate;

activated carbon-supported thiazolium hydroxide potassium sulfonate;

activated carbon-supported pyridinium hydroxide potassium sulfonate;

activated carbon-supported pyrimidinium hydroxide potassium sulfonate;

activated carbon-supported pyrazinium hydroxide potassium sulfonate;

activated carbon-supported pyradizimium hydroxide potassium sulfonate;

activated carbon-supported thiazinium hydroxide potassium sulfonate;

activated carbon-supported morpholinium hydroxide potassium sulfonate;

activated carbon-supported piperidinium hydroxide potassium sulfonate;

activated carbon-supported piperizinium hydroxide potassium sulfonate;

activated carbon-supported pyrollizinium hydroxide potassium sulfonate;

activated carbon-supported triphenyl phosphonium hydroxide potassium sulfonate;

activated carbon-supported trimethyl phosphonium hydroxide potassium sulfonate;

activated carbon-supported triethyl phosphonium hydroxide potassium sulfonate;

activated carbon-supported tripropyl phosphonium hydroxide potassium sulfonate;

activated carbon-supported tributyl phosphonium hydroxide potassium sulfonate;

activated carbon-supported trifluoro phosphonium hydroxide potassium sulfonate;

activated carbon-supported sulfonium hydroxide potassium sulfonate;

activated carbon-supported methylsulfonium hydroxide potassium sulfonate;

activated carbon-supported dimethylsulfonium hydroxide potassium sulfonate;

activated carbon-supported trimethylsulfonium hydroxide potassium sulfonate;

activated carbon-supported tetramethylsulfonium hydroxide potassium sulfonate;

activated carbon-supported ethylsulfonium hydroxide potassium sulfonate;

activated carbon-supported diethylsulfonium hydroxide potassium sulfonate;

activated carbon-supported triethylsulfonium hydroxide potassium sulfonate;

activated carbon-supported tetraethylsulfonium hydroxide potassium sulfonate;

activated carbon-supported propylsulfonium hydroxide potassium sulfonate;

activated carbon-supported dipropylsulfonium hydroxide potassium sulfonate;

activated carbon-supported tripropylsulfonium hydroxide potassium sulfonate;

activated carbon-supported tetrapropylsulfonium hydroxide potassium sulfonate;

activated carbon-supported phenylsulfonium hydroxide potassium sulfonate;

activated carbon-supported diphenylsulfonium hydroxide potassium sulfonate;

activated carbon-supported triphenylsulfonium hydroxide potassium sulfonate;

activated carbon-supported tetraphenylsulfonium hydroxide potassium sulfonate;

activated carbon-supported pyrrolium hydroxide magnesium sulfonate;

activated carbon-supported imidazolium hydroxide magnesium sulfonate;

activated carbon-supported pyrazolium hydroxide magnesium sulfonate;

activated carbon-supported oxazolium hydroxide magnesium sulfonate;

activated carbon-supported thiazolium hydroxide magnesium sulfonate;

activated carbon-supported pyridinium hydroxide magnesium sulfonate;

activated carbon-supported pyrimidinium hydroxide magnesium sulfonate;

activated carbon-supported pyrazinium hydroxide magnesium sulfonate;

activated carbon-supported pyradizimium hydroxide magnesium sulfonate;

activated carbon-supported thiazinium hydroxide magnesium sulfonate;

activated carbon-supported morpholinium hydroxide magnesium sulfonate;

activated carbon-supported piperidinium hydroxide magnesium sulfonate;

activated carbon-supported piperizinium hydroxide magnesium sulfonate;

activated carbon-supported pyrollizinium hydroxide magnesium sulfonate;

activated carbon-supported triphenyl phosphonium hydroxide magnesium sulfonate;

activated carbon-supported trimethyl phosphonium hydroxide magnesium sulfonate;

activated carbon-supported triethyl phosphonium hydroxide magnesium sulfonate;

activated carbon-supported tripropyl phosphonium hydroxide magnesium sulfonate;

activated carbon-supported tributyl phosphonium hydroxide magnesium sulfonate;

activated carbon-supported trifluoro phosphonium hydroxide magnesium sulfonate;

activated carbon-supported sulfonium hydroxide magnesium sulfonate;

activated carbon-supported methylsulfonium hydroxide magnesium sulfonate;

activated carbon-supported dimethylsulfonium hydroxide magnesium sulfonate;

activated carbon-supported trimethylsulfonium hydroxide magnesium sulfonate;

activated carbon-supported tetramethylsulfonium hydroxide magnesium sulfonate;

activated carbon-supported ethylsulfonium hydroxide magnesium sulfonate;

activated carbon-supported diethylsulfonium hydroxide magnesium sulfonate;

activated carbon-supported triethylsulfonium hydroxide magnesium sulfonate;

activated carbon-supported tetraethylsulfonium hydroxide magnesium sulfonate;

activated carbon-supported propylsulfonium hydroxide magnesium sulfonate;

activated carbon-supported dipropylsulfonium hydroxide magnesium sulfonate;

activated carbon-supported tripropylsulfonium hydroxide magnesium sulfonate;

activated carbon-supported tetrapropylsulfonium hydroxide magnesium sulfonate;

activated carbon-supported phenylsulfonium hydroxide magnesium sulfonate;

activated carbon-supported diphenylsulfonium hydroxide magnesium sulfonate;

activated carbon-supported triphenylsulfonium hydroxide magnesium sulfonate;

activated carbon-supported tetraphenylsulfonium hydroxide magnesium sulfonate;

activated carbon-supported pyrrolium hydroxide calcium sulfonate;

activated carbon-supported imidazolium hydroxide calcium sulfonate;

activated carbon-supported pyrazolium hydroxide calcium sulfonate;

activated carbon-supported oxazolium hydroxide calcium sulfonate;

activated carbon-supported thiazolium hydroxide calcium sulfonate;

activated carbon-supported pyridinium hydroxide calcium sulfonate;

activated carbon-supported pyrimidinium hydroxide calcium sulfonate;

activated carbon-supported pyrazinium hydroxide calcium sulfonate;

activated carbon-supported pyradizimium hydroxide calcium sulfonate;

activated carbon-supported thiazinium hydroxide calcium sulfonate;

activated carbon-supported morpholinium hydroxide calcium sulfonate;

activated carbon-supported piperidinium hydroxide calcium sulfonate;

activated carbon-supported piperizinium hydroxide calcium sulfonate;

activated carbon-supported pyrollizinium hydroxide calcium sulfonate;

activated carbon-supported triphenyl phosphonium hydroxide calcium sulfonate;

activated carbon-supported trimethyl phosphonium hydroxide calcium sulfonate;

activated carbon-supported triethyl phosphonium hydroxide calcium sulfonate;

activated carbon-supported tripropyl phosphonium hydroxide calcium sulfonate;

activated carbon-supported tributyl phosphonium hydroxide calcium sulfonate;

activated carbon-supported trifluoro phosphonium hydroxide calcium sulfonate;

activated carbon-supported sulfonium hydroxide calcium sulfonate;

activated carbon-supported methylsulfonium hydroxide calcium sulfonate;

activated carbon-supported dimethylsulfonium hydroxide calcium sulfonate;

activated carbon-supported trimethylsulfonium hydroxide calcium sulfonate;

activated carbon-supported tetramethylsulfonium hydroxide calcium sulfonate;

activated carbon-supported ethylsulfonium hydroxide calcium sulfonate;

activated carbon-supported diethylsulfonium hydroxide calcium sulfonate;

activated carbon-supported triethylsulfonium hydroxide calcium sulfonate;

activated carbon-supported tetraethylsulfonium hydroxide calcium sulfonate;

activated carbon-supported propylsulfonium hydroxide calcium sulfonate;

activated carbon-supported dipropylsulfonium hydroxide calcium sulfonate;

activated carbon-supported tripropylsulfonium hydroxide calcium sulfonate;

activated carbon-supported tetrapropylsulfonium hydroxide calcium sulfonate;

activated carbon-supported phenylsulfonium hydroxide calcium sulfonate;

activated carbon-supported diphenylsulfonium hydroxide calcium sulfonate;

activated carbon-supported triphenylsulfonium hydroxide calcium sulfonate;

activated carbon-supported tetraphenylsulfonium hydroxide calcium sulfonate;

activated carbon-supported pyrrolium hydroxide sodium phosphonate;

activated carbon-supported imidazolium hydroxide sodium phosphonate;

activated carbon-supported pyrazolium hydroxide sodium phosphonate;

activated carbon-supported oxazolium hydroxide sodium phosphonate;

activated carbon-supported thiazolium hydroxide sodium phosphonate;

activated carbon-supported pyridinium hydroxide sodium phosphonate;

activated carbon-supported pyrimidinium hydroxide sodium phosphonate;

activated carbon-supported pyrazinium hydroxide sodium phosphonate;

activated carbon-supported pyradizimium hydroxide sodium phosphonate;

activated carbon-supported thiazinium hydroxide sodium phosphonate;

activated carbon-supported morpholinium hydroxide sodium phosphonate;

activated carbon-supported piperidinium hydroxide sodium phosphonate;

activated carbon-supported piperizinium hydroxide sodium phosphonate;

activated carbon-supported pyrollizinium hydroxide sodium phosphonate;

activated carbon-supported triphenyl phosphonium hydroxide sodium phosphonate;

activated carbon-supported trimethyl phosphonium hydroxide sodium phosphonate;

activated carbon-supported triethyl phosphonium hydroxide sodium phosphonate;

activated carbon-supported tripropyl phosphonium hydroxide sodium phosphonate;

activated carbon-supported tributyl phosphonium hydroxide sodium phosphonate;

activated carbon-supported trifluoro phosphonium hydroxide sodium phosphonate;

activated carbon-supported sulfonium hydroxide sodium phosphonate;

activated carbon-supported methylsulfonium hydroxide sodium phosphonate;

activated carbon-supported dimethylsulfonium hydroxide sodium phosphonate;

activated carbon-supported trimethylsulfonium hydroxide sodium phosphonate;

activated carbon-supported tetramethylsulfonium hydroxide sodium phosphonate;

activated carbon-supported ethylsulfonium hydroxide sodium phosphonate;

activated carbon-supported diethylsulfonium hydroxide sodium phosphonate;

activated carbon-supported triethylsulfonium hydroxide sodium phosphonate;

activated carbon-supported tetraethylsulfonium hydroxide sodium phosphonate;

activated carbon-supported propylsulfonium hydroxide sodium phosphonate;

activated carbon-supported dipropylsulfonium hydroxide sodium phosphonate;

activated carbon-supported tripropylsulfonium hydroxide sodium phosphonate;

activated carbon-supported tetrapropylsulfonium hydroxide sodium phosphonate;

activated carbon-supported phenylsulfonium hydroxide sodium phosphonate;

activated carbon-supported diphenylsulfonium hydroxide sodium phosphonate;

activated carbon-supported triphenylsulfonium hydroxide sodium phosphonate;

activated carbon-supported tetraphenylsulfonium hydroxide sodium phosphonate;

activated carbon-supported pyrrolium hydroxide potassium phosphonate;

activated carbon-supported imidazolium hydroxide potassium phosphonate;

activated carbon-supported pyrazolium hydroxide potassium phosphonate;

activated carbon-supported oxazolium hydroxide potassium phosphonate;

activated carbon-supported thiazolium hydroxide potassium phosphonate;

activated carbon-supported pyridinium hydroxide potassium phosphonate;

activated carbon-supported pyrimidinium hydroxide potassium phosphonate;

activated carbon-supported pyrazinium hydroxide potassium phosphonate;

activated carbon-supported pyradizimium hydroxide potassium phosphonate;

activated carbon-supported thiazinium hydroxide potassium phosphonate;

activated carbon-supported morpholinium hydroxide potassium phosphonate;

activated carbon-supported piperidinium hydroxide potassium phosphonate;

activated carbon-supported piperizinium hydroxide potassium phosphonate;

activated carbon-supported pyrollizinium hydroxide potassium phosphonate;

activated carbon-supported triphenyl phosphonium hydroxide potassium phosphonate;

activated carbon-supported trimethyl phosphonium hydroxide potassium phosphonate;

activated carbon-supported triethyl phosphonium hydroxide potassium phosphonate;

activated carbon-supported tripropyl phosphonium hydroxide potassium phosphonate;

activated carbon-supported tributyl phosphonium hydroxide potassium phosphonate;

activated carbon-supported trifluoro phosphonium hydroxide potassium phosphonate;

activated carbon-supported sulfonium hydroxide potassium phosphonate;

activated carbon-supported methylsulfonium hydroxide potassium phosphonate;

activated carbon-supported dimethylsulfonium hydroxide potassium phosphonate;

activated carbon-supported trimethylsulfonium hydroxide potassium phosphonate;

activated carbon-supported tetramethylsulfonium hydroxide potassium phosphonate;

activated carbon-supported ethylsulfonium hydroxide potassium phosphonate;

activated carbon-supported diethylsulfonium hydroxide potassium phosphonate;

activated carbon-supported triethylsulfonium hydroxide potassium phosphonate;

activated carbon-supported tetraethylsulfonium hydroxide potassium phosphonate;

activated carbon-supported propylsulfonium hydroxide potassium phosphonate;

activated carbon-supported dipropylsulfonium hydroxide potassium phosphonate;

activated carbon-supported tripropylsulfonium hydroxide potassium phosphonate;

activated carbon-supported tetrapropylsulfonium hydroxide potassium phosphonate;

activated carbon-supported phenylsulfonium hydroxide potassium phosphonate;

activated carbon-supported diphenylsulfonium hydroxide potassium phosphonate;

activated carbon-supported triphenylsulfonium hydroxide potassium phosphonate;

activated carbon-supported tetraphenylsulfonium hydroxide potassium phosphonate;

activated carbon-supported pyrrolium hydroxide magnesium phosphonate;

activated carbon-supported imidazolium hydroxide magnesium phosphonate;

activated carbon-supported pyrazolium hydroxide magnesium phosphonate;

activated carbon-supported oxazolium hydroxide magnesium phosphonate;

activated carbon-supported thiazolium hydroxide magnesium phosphonate;

activated carbon-supported pyridinium hydroxide magnesium phosphonate;

activated carbon-supported pyrimidinium hydroxide magnesium phosphonate;

activated carbon-supported pyrazinium hydroxide magnesium phosphonate;

activated carbon-supported pyradizimium hydroxide magnesium phosphonate;

activated carbon-supported thiazinium hydroxide magnesium phosphonate;

activated carbon-supported morpholinium hydroxide magnesium phosphonate;

activated carbon-supported piperidinium hydroxide magnesium phosphonate;

activated carbon-supported piperizinium hydroxide magnesium phosphonate;

activated carbon-supported pyrollizinium hydroxide magnesium phosphonate;

activated carbon-supported triphenyl phosphonium hydroxide magnesium phosphonate;

activated carbon-supported trimethyl phosphonium hydroxide magnesium phosphonate;

activated carbon-supported triethyl phosphonium hydroxide magnesium phosphonate;

activated carbon-supported tripropyl phosphonium hydroxide magnesium phosphonate;

activated carbon-supported tributyl phosphonium hydroxide magnesium phosphonate;

activated carbon-supported trifluoro phosphonium hydroxide magnesium phosphonate;

activated carbon-supported sulfonium hydroxide magnesium phosphonate;

activated carbon-supported methylsulfonium hydroxide magnesium phosphonate;

activated carbon-supported dimethylsulfonium hydroxide magnesium phosphonate;

activated carbon-supported trimethylsulfonium hydroxide magnesium phosphonate;

activated carbon-supported tetramethylsulfonium hydroxide magnesium phosphonate;

activated carbon-supported ethylsulfonium hydroxide magnesium phosphonate;

activated carbon-supported diethylsulfonium hydroxide magnesium phosphonate;

activated carbon-supported triethylsulfonium hydroxide magnesium phosphonate;

activated carbon-supported tetraethylsulfonium hydroxide magnesium phosphonate;

activated carbon-supported propylsulfonium hydroxide magnesium phosphonate;

activated carbon-supported dipropylsulfonium hydroxide magnesium phosphonate;

activated carbon-supported tripropylsulfonium hydroxide magnesium phosphonate;

activated carbon-supported tetrapropylsulfonium hydroxide magnesium phosphonate;

activated carbon-supported phenylsulfonium hydroxide magnesium phosphonate;

activated carbon-supported diphenylsulfonium hydroxide magnesium phosphonate;

activated carbon-supported triphenylsulfonium hydroxide magnesium phosphonate;

activated carbon-supported tetraphenylsulfonium hydroxide magnesium phosphonate;

activated carbon-supported pyrrolium hydroxide calcium phosphonate;

activated carbon-supported imidazolium hydroxide calcium phosphonate;

activated carbon-supported pyrazolium hydroxide calcium phosphonate;

activated carbon-supported oxazolium hydroxide calcium phosphonate;

activated carbon-supported thiazolium hydroxide calcium phosphonate;

activated carbon-supported pyridinium hydroxide calcium phosphonate;

activated carbon-supported pyrimidinium hydroxide calcium phosphonate;

activated carbon-supported pyrazinium hydroxide calcium phosphonate;

activated carbon-supported pyradizimium hydroxide calcium phosphonate;

activated carbon-supported thiazinium hydroxide calcium phosphonate;

activated carbon-supported morpholinium hydroxide calcium phosphonate;

activated carbon-supported piperidinium hydroxide calcium phosphonate;

activated carbon-supported piperizinium hydroxide calcium phosphonate;

activated carbon-supported pyrollizinium hydroxide calcium phosphonate;

activated carbon-supported triphenyl phosphonium hydroxide calcium phosphonate;

activated carbon-supported trimethyl phosphonium hydroxide calcium phosphonate;

activated carbon-supported triethyl phosphonium hydroxide calcium phosphonate;

activated carbon-supported tripropyl phosphonium hydroxide calcium phosphonate;

activated carbon-supported tributyl phosphonium hydroxide calcium phosphonate;

activated carbon-supported trifluoro phosphonium hydroxide calcium phosphonate;

activated carbon-supported sulfonium hydroxide calcium phosphonate;

activated carbon-supported methylsulfonium hydroxide calcium phosphonate;

activated carbon-supported dimethylsulfonium hydroxide calcium phosphonate;

activated carbon-supported trimethylsulfonium hydroxide calcium phosphonate;

activated carbon-supported tetramethylsulfonium hydroxide calcium phosphonate;

activated carbon-supported ethylsulfonium hydroxide calcium phosphonate;

activated carbon-supported diethylsulfonium hydroxide calcium phosphonate;

activated carbon-supported triethylsulfonium hydroxide calcium phosphonate;

activated carbon-supported tetraethylsulfonium hydroxide calcium phosphonate;

activated carbon-supported propylsulfonium hydroxide calcium phosphonate;

activated carbon-supported dipropylsulfonium hydroxide calcium phosphonate;

activated carbon-supported tripropylsulfonium hydroxide calcium phosphonate;

activated carbon-supported tetrapropylsulfonium hydroxide calcium phosphonate;

activated carbon-supported phenylsulfonium hydroxide calcium phosphonate;

activated carbon-supported diphenylsulfonium hydroxide calcium phosphonate;

activated carbon-supported triphenylsulfonium hydroxide calcium phosphonate;

activated carbon-supported tetraphenylsulfonium hydroxide calcium phosphonate;

activated carbon-supported pyrrolium hydroxide sodium acetate;

activated carbon-supported imidazolium hydroxide sodium acetate;

activated carbon-supported pyrazolium hydroxide sodium acetate;

activated carbon-supported oxazolium hydroxide sodium acetate;

activated carbon-supported thiazolium hydroxide sodium acetate;

activated carbon-supported pyridinium hydroxide sodium acetate;

activated carbon-supported pyrimidinium hydroxide sodium acetate;

activated carbon-supported pyrazinium hydroxide sodium acetate;

activated carbon-supported pyradizimium hydroxide sodium acetate;

activated carbon-supported thiazinium hydroxide sodium acetate;

activated carbon-supported morpholinium hydroxide sodium acetate;

activated carbon-supported piperidinium hydroxide sodium acetate;

activated carbon-supported piperizinium hydroxide sodium acetate;

activated carbon-supported pyrollizinium hydroxide sodium acetate;

activated carbon-supported triphenyl phosphonium hydroxide sodium acetate;

activated carbon-supported trimethyl phosphonium hydroxide sodium acetate;

activated carbon-supported triethyl phosphonium hydroxide sodium acetate;

activated carbon-supported tripropyl phosphonium hydroxide sodium acetate;

activated carbon-supported tributyl phosphonium hydroxide sodium acetate;

activated carbon-supported trifluoro phosphonium hydroxide sodium acetate;

activated carbon-supported sulfonium hydroxide sodium acetate;

activated carbon-supported methylsulfonium hydroxide sodium acetate;

activated carbon-supported dimethylsulfonium hydroxide sodium acetate;

activated carbon-supported trimethylsulfonium hydroxide sodium acetate;

activated carbon-supported tetramethylsulfonium hydroxide sodium acetate;

activated carbon-supported ethylsulfonium hydroxide sodium acetate;

activated carbon-supported diethylsulfonium hydroxide sodium acetate;

activated carbon-supported triethylsulfonium hydroxide sodium acetate;

activated carbon-supported tetraethylsulfonium hydroxide sodium acetate;

activated carbon-supported propylsulfonium hydroxide sodium acetate;

activated carbon-supported dipropylsulfonium hydroxide sodium acetate;

activated carbon-supported tripropylsulfonium hydroxide sodium acetate;

activated carbon-supported tetrapropylsulfonium hydroxide sodium acetate;

activated carbon-supported phenylsulfonium hydroxide sodium acetate;

activated carbon-supported diphenylsulfonium hydroxide sodium acetate;

activated carbon-supported triphenylsulfonium hydroxide sodium acetate;

activated carbon-supported tetraphenylsulfonium hydroxide sodium acetate;

activated carbon-supported pyrrolium hydroxide potassium acetate;

activated carbon-supported imidazolium hydroxide potassium acetate;

activated carbon-supported pyrazolium hydroxide potassium acetate;

activated carbon-supported oxazolium hydroxide potassium acetate;

activated carbon-supported thiazolium hydroxide potassium acetate;

activated carbon-supported pyridinium hydroxide potassium acetate;

activated carbon-supported pyrimidinium hydroxide potassium acetate;

activated carbon-supported pyrazinium hydroxide potassium acetate;

activated carbon-supported pyradizimium hydroxide potassium acetate;

activated carbon-supported thiazinium hydroxide potassium acetate;

activated carbon-supported morpholinium hydroxide potassium acetate;

activated carbon-supported piperidinium hydroxide potassium acetate;

activated carbon-supported piperizinium hydroxide potassium acetate;

activated carbon-supported pyrollizinium hydroxide potassium acetate;

activated carbon-supported triphenyl phosphonium hydroxide potassium acetate;

activated carbon-supported trimethyl phosphonium hydroxide potassium acetate;

activated carbon-supported triethyl phosphonium hydroxide potassium acetate;

activated carbon-supported tripropyl phosphonium hydroxide potassium acetate;

activated carbon-supported tributyl phosphonium hydroxide potassium acetate;

activated carbon-supported trifluoro phosphonium hydroxide potassium acetate;

activated carbon-supported sulfonium hydroxide potassium acetate;

activated carbon-supported methylsulfonium hydroxide potassium acetate;

activated carbon-supported dimethylsulfonium hydroxide potassium acetate;

activated carbon-supported trimethylsulfonium hydroxide potassium acetate;

activated carbon-supported tetramethylsulfonium hydroxide potassium acetate;

activated carbon-supported ethylsulfonium hydroxide potassium acetate;

activated carbon-supported diethylsulfonium hydroxide potassium acetate;

activated carbon-supported triethylsulfonium hydroxide potassium acetate;

activated carbon-supported tetraethylsulfonium hydroxide potassium acetate;

activated carbon-supported propylsulfonium hydroxide potassium acetate;

activated carbon-supported dipropylsulfonium hydroxide potassium acetate;

activated carbon-supported tripropylsulfonium hydroxide potassium acetate;

activated carbon-supported tetrapropylsulfonium hydroxide potassium acetate;

activated carbon-supported phenylsulfonium hydroxide potassium acetate;

activated carbon-supported diphenylsulfonium hydroxide potassium acetate;

activated carbon-supported triphenylsulfonium hydroxide potassium acetate;

activated carbon-supported tetraphenylsulfonium hydroxide potassium acetate;

activated carbon-supported pyrrolium hydroxide magnesium acetate;

activated carbon-supported imidazolium hydroxide magnesium acetate;

activated carbon-supported pyrazolium hydroxide magnesium acetate;

activated carbon-supported oxazolium hydroxide magnesium acetate;

activated carbon-supported thiazolium hydroxide magnesium acetate;

activated carbon-supported pyridinium hydroxide magnesium acetate;

activated carbon-supported pyrimidinium hydroxide magnesium acetate;

activated carbon-supported pyrazinium hydroxide magnesium acetate;

activated carbon-supported pyradizimium hydroxide magnesium acetate;

activated carbon-supported thiazinium hydroxide magnesium acetate;

activated carbon-supported morpholinium hydroxide magnesium acetate;

activated carbon-supported piperidinium hydroxide magnesium acetate;

activated carbon-supported piperizinium hydroxide magnesium acetate;

activated carbon-supported pyrollizinium hydroxide magnesium acetate;

activated carbon-supported triphenyl phosphonium hydroxide magnesium acetate;

activated carbon-supported trimethyl phosphonium hydroxide magnesium acetate;

activated carbon-supported triethyl phosphonium hydroxide magnesium acetate;

activated carbon-supported tripropyl phosphonium hydroxide magnesium acetate;

activated carbon-supported tributyl phosphonium hydroxide magnesium acetate;

activated carbon-supported trifluoro phosphonium hydroxide magnesium acetate;

activated carbon-supported sulfonium hydroxide magnesium acetate;

activated carbon-supported methylsulfonium hydroxide magnesium acetate;

activated carbon-supported dimethylsulfonium hydroxide magnesium acetate;

activated carbon-supported trimethylsulfonium hydroxide magnesium acetate;

activated carbon-supported tetramethylsulfonium hydroxide magnesium acetate;

activated carbon-supported ethylsulfonium hydroxide magnesium acetate;

activated carbon-supported diethylsulfonium hydroxide magnesium acetate;

activated carbon-supported triethylsulfonium hydroxide magnesium acetate;

activated carbon-supported tetraethylsulfonium hydroxide magnesium acetate;

activated carbon-supported propylsulfonium hydroxide magnesium acetate;

activated carbon-supported dipropylsulfonium hydroxide magnesium acetate;

activated carbon-supported tripropylsulfonium hydroxide magnesium acetate;

activated carbon-supported tetrapropylsulfonium hydroxide magnesium acetate;

activated carbon-supported phenylsulfonium hydroxide magnesium acetate;

activated carbon-supported diphenylsulfonium hydroxide magnesium acetate;

activated carbon-supported triphenylsulfonium hydroxide magnesium acetate;

activated carbon-supported tetraphenylsulfonium hydroxide magnesium acetate;

activated carbon-supported pyrrolium hydroxide calcium acetate;

activated carbon-supported imidazolium hydroxide calcium acetate;

activated carbon-supported pyrazolium hydroxide calcium acetate;

activated carbon-supported oxazolium hydroxide calcium acetate;

activated carbon-supported thiazolium hydroxide calcium acetate;

activated carbon-supported pyridinium hydroxide calcium acetate;

activated carbon-supported pyrimidinium hydroxide calcium acetate;

activated carbon-supported pyrazinium hydroxide calcium acetate;

activated carbon-supported pyradizimium hydroxide calcium acetate;

activated carbon-supported thiazinium hydroxide calcium acetate;

activated carbon-supported morpholinium hydroxide calcium acetate;

activated carbon-supported piperidinium hydroxide calcium acetate;

activated carbon-supported piperizinium hydroxide calcium acetate;

activated carbon-supported pyrollizinium hydroxide calcium acetate;

activated carbon-supported triphenyl phosphonium hydroxide calcium acetate;

activated carbon-supported trimethyl phosphonium hydroxide calcium acetate;

activated carbon-supported triethyl phosphonium hydroxide calcium acetate;

activated carbon-supported tripropyl phosphonium hydroxide calcium acetate;

activated carbon-supported tributyl phosphonium hydroxide calcium acetate;

activated carbon-supported trifluoro phosphonium hydroxide calcium acetate;

activated carbon-supported sulfonium hydroxide calcium acetate;

activated carbon-supported methylsulfonium hydroxide calcium acetate;

activated carbon-supported dimethylsulfonium hydroxide calcium acetate;

activated carbon-supported trimethylsulfonium hydroxide calcium acetate;

activated carbon-supported tetramethylsulfonium hydroxide calcium acetate;

activated carbon-supported ethylsulfonium hydroxide calcium acetate;

activated carbon-supported diethylsulfonium hydroxide calcium acetate;

activated carbon-supported triethylsulfonium hydroxide calcium acetate;

activated carbon-supported tetraethylsulfonium hydroxide calcium acetate;

activated carbon-supported propylsulfonium hydroxide calcium acetate;

activated carbon-supported dipropylsulfonium hydroxide calcium acetate;

activated carbon-supported tripropylsulfonium hydroxide calcium acetate;

activated carbon-supported tetrapropylsulfonium hydroxide calcium acetate;

activated carbon-supported phenylsulfonium hydroxide calcium acetate;

activated carbon-supported diphenylsulfonium hydroxide calcium acetate;

activated carbon-supported triphenylsulfonium hydroxide calcium acetate;

activated carbon-supported tetraphenylsulfonium hydroxide calcium acetate;

activated carbon-supported pyrrolium hydroxide sodium isophthalate;

activated carbon-supported imidazolium hydroxide sodium isophthalate;

activated carbon-supported pyrazolium hydroxide sodium isophthalate;

activated carbon-supported oxazolium hydroxide sodium isophthalate;

activated carbon-supported thiazolium hydroxide sodium isophthalate;

activated carbon-supported pyridinium hydroxide sodium isophthalate;

activated carbon-supported pyrimidinium hydroxide sodium isophthalate;

activated carbon-supported pyrazinium hydroxide sodium isophthalate;

activated carbon-supported pyradizimium hydroxide sodium isophthalate;

activated carbon-supported thiazinium hydroxide sodium isophthalate;

activated carbon-supported morpholinium hydroxide sodium isophthalate;

activated carbon-supported piperidinium hydroxide sodium isophthalate;

activated carbon-supported piperizinium hydroxide sodium isophthalate;

activated carbon-supported pyrollizinium hydroxide sodium isophthalate;

activated carbon-supported triphenyl phosphonium hydroxide sodium isophthalate;

activated carbon-supported trimethyl phosphonium hydroxide sodium isophthalate;

activated carbon-supported triethyl phosphonium hydroxide sodium isophthalate;

activated carbon-supported tripropyl phosphonium hydroxide sodium isophthalate;

activated carbon-supported tributyl phosphonium hydroxide sodium isophthalate;

activated carbon-supported trifluoro phosphonium hydroxide sodium isophthalate;

activated carbon-supported sulfonium hydroxide sodium isophthalate;

activated carbon-supported methylsulfonium hydroxide sodium isophthalate;

activated carbon-supported dimethylsulfonium hydroxide sodium isophthalate;

activated carbon-supported trimethylsulfonium hydroxide sodium isophthalate;

activated carbon-supported tetramethylsulfonium hydroxide sodium isophthalate;

activated carbon-supported ethylsulfonium hydroxide sodium isophthalate;

activated carbon-supported diethylsulfonium hydroxide sodium isophthalate;

activated carbon-supported triethylsulfonium hydroxide sodium isophthalate;

activated carbon-supported tetraethylsulfonium hydroxide sodium isophthalate;

activated carbon-supported propylsulfonium hydroxide sodium isophthalate;

activated carbon-supported dipropylsulfonium hydroxide sodium isophthalate;

activated carbon-supported tripropylsulfonium hydroxide sodium isophthalate;

activated carbon-supported tetrapropylsulfonium hydroxide sodium isophthalate;

activated carbon-supported phenylsulfonium hydroxide sodium isophthalate;

activated carbon-supported diphenylsulfonium hydroxide sodium isophthalate;

activated carbon-supported triphenylsulfonium hydroxide sodium isophthalate;

activated carbon-supported tetraphenylsulfonium hydroxide sodium isophthalate;

activated carbon-supported pyrrolium hydroxide potassium isophthalate;

activated carbon-supported imidazolium hydroxide potassium isophthalate;

activated carbon-supported pyrazolium hydroxide potassium isophthalate;

activated carbon-supported oxazolium hydroxide potassium isophthalate;

activated carbon-supported thiazolium hydroxide potassium isophthalate;

activated carbon-supported pyridinium hydroxide potassium isophthalate;

activated carbon-supported pyrimidinium hydroxide potassium isophthalate;

activated carbon-supported pyrazinium hydroxide potassium isophthalate;

activated carbon-supported pyradizimium hydroxide potassium isophthalate;

activated carbon-supported thiazinium hydroxide potassium isophthalate;

activated carbon-supported morpholinium hydroxide potassium isophthalate;

activated carbon-supported piperidinium hydroxide potassium isophthalate;

activated carbon-supported piperizinium hydroxide potassium isophthalate;

activated carbon-supported pyrollizinium hydroxide potassium isophthalate;

activated carbon-supported triphenyl phosphonium hydroxide potassium isophthalate;

activated carbon-supported trimethyl phosphonium hydroxide potassium isophthalate;

activated carbon-supported triethyl phosphonium hydroxide potassium isophthalate;

activated carbon-supported tripropyl phosphonium hydroxide potassium isophthalate;

activated carbon-supported tributyl phosphonium hydroxide potassium isophthalate;

activated carbon-supported trifluoro phosphonium hydroxide potassium isophthalate;

activated carbon-supported sulfonium hydroxide potassium isophthalate;

activated carbon-supported methylsulfonium hydroxide potassium isophthalate;

activated carbon-supported dimethylsulfonium hydroxide potassium isophthalate;

activated carbon-supported trimethylsulfonium hydroxide potassium isophthalate;

activated carbon-supported tetramethylsulfonium hydroxide potassium isophthalate;

activated carbon-supported ethylsulfonium hydroxide potassium isophthalate;

activated carbon-supported diethylsulfonium hydroxide potassium isophthalate;

activated carbon-supported triethylsulfonium hydroxide potassium isophthalate;

activated carbon-supported tetraethylsulfonium hydroxide potassium isophthalate;

activated carbon-supported propylsulfonium hydroxide potassium isophthalate;

activated carbon-supported dipropylsulfonium hydroxide potassium isophthalate;

activated carbon-supported tripropylsulfonium hydroxide potassium isophthalate;

activated carbon-supported tetrapropylsulfonium hydroxide potassium isophthalate;

activated carbon-supported phenylsulfonium hydroxide potassium isophthalate;

activated carbon-supported diphenylsulfonium hydroxide potassium isophthalate;

activated carbon-supported triphenylsulfonium hydroxide potassium isophthalate;

activated carbon-supported tetraphenylsulfonium hydroxide potassium isophthalate;

activated carbon-supported pyrrolium hydroxide magnesium isophthalate;

activated carbon-supported imidazolium hydroxide magnesium isophthalate;

activated carbon-supported pyrazolium hydroxide magnesium isophthalate;

activated carbon-supported oxazolium hydroxide magnesium isophthalate;

activated carbon-supported thiazolium hydroxide magnesium isophthalate;

activated carbon-supported pyridinium hydroxide magnesium isophthalate;

activated carbon-supported pyrimidinium hydroxide magnesium isophthalate;

activated carbon-supported pyrazinium hydroxide magnesium isophthalate;

activated carbon-supported pyradizimium hydroxide magnesium isophthalate;

activated carbon-supported thiazinium hydroxide magnesium isophthalate;

activated carbon-supported morpholinium hydroxide magnesium isophthalate;

activated carbon-supported piperidinium hydroxide magnesium isophthalate;

activated carbon-supported piperizinium hydroxide magnesium isophthalate;

activated carbon-supported pyrollizinium hydroxide magnesium isophthalate;

activated carbon-supported triphenyl phosphonium hydroxide magnesium isophthalate;

activated carbon-supported trimethyl phosphonium hydroxide magnesium isophthalate;

activated carbon-supported triethyl phosphonium hydroxide magnesium isophthalate;

activated carbon-supported tripropyl phosphonium hydroxide magnesium isophthalate;

activated carbon-supported tributyl phosphonium hydroxide magnesium isophthalate;

activated carbon-supported trifluoro phosphonium hydroxide magnesium isophthalate;

activated carbon-supported sulfonium hydroxide magnesium isophthalate;

activated carbon-supported methylsulfonium hydroxide magnesium isophthalate;

activated carbon-supported dimethylsulfonium hydroxide magnesium isophthalate;

activated carbon-supported trimethylsulfonium hydroxide magnesium isophthalate;

activated carbon-supported tetramethylsulfonium hydroxide magnesium isophthalate;

activated carbon-supported ethylsulfonium hydroxide magnesium isophthalate;

activated carbon-supported diethylsulfonium hydroxide magnesium isophthalate;

activated carbon-supported triethylsulfonium hydroxide magnesium isophthalate;

activated carbon-supported tetraethylsulfonium hydroxide magnesium isophthalate;

activated carbon-supported propylsulfonium hydroxide magnesium isophthalate;

activated carbon-supported dipropylsulfonium hydroxide magnesium isophthalate;

activated carbon-supported tripropylsulfonium hydroxide magnesium isophthalate;

activated carbon-supported tetrapropylsulfonium hydroxide magnesium isophthalate;

activated carbon-supported phenylsulfonium hydroxide magnesium isophthalate;

activated carbon-supported diphenylsulfonium hydroxide magnesium isophthalate;

activated carbon-supported triphenylsulfonium hydroxide magnesium isophthalate;

activated carbon-supported tetraphenylsulfonium hydroxide magnesium isophthalate;

activated carbon-supported pyrrolium hydroxide calcium isophthalate;

activated carbon-supported imidazolium hydroxide calcium isophthalate;

activated carbon-supported pyrazolium hydroxide calcium isophthalate;

activated carbon-supported oxazolium hydroxide calcium isophthalate;

activated carbon-supported thiazolium hydroxide calcium isophthalate;

activated carbon-supported pyridinium hydroxide calcium isophthalate;

activated carbon-supported pyrimidinium hydroxide calcium isophthalate;

activated carbon-supported pyrazinium hydroxide calcium isophthalate;

activated carbon-supported pyradizimium hydroxide calcium isophthalate;

activated carbon-supported thiazinium hydroxide calcium isophthalate;

activated carbon-supported morpholinium hydroxide calcium isophthalate;

activated carbon-supported piperidinium hydroxide calcium isophthalate;

activated carbon-supported piperizinium hydroxide calcium isophthalate;

activated carbon-supported pyrollizinium hydroxide calcium isophthalate;

activated carbon-supported triphenyl phosphonium hydroxide calcium isophthalate;

activated carbon-supported trimethyl phosphonium hydroxide calcium isophthalate;

activated carbon-supported triethyl phosphonium hydroxide calcium isophthalate;

activated carbon-supported tripropyl phosphonium hydroxide calcium isophthalate;

activated carbon-supported tributyl phosphonium hydroxide calcium isophthalate;

activated carbon-supported trifluoro phosphonium hydroxide calcium isophthalate;

activated carbon-supported sulfonium hydroxide calcium isophthalate;

activated carbon-supported methylsulfonium hydroxide calcium isophthalate;

activated carbon-supported dimethylsulfonium hydroxide calcium isophthalate;

activated carbon-supported trimethylsulfonium hydroxide calcium isophthalate;

activated carbon-supported tetramethylsulfonium hydroxide calcium isophthalate;

activated carbon-supported ethylsulfonium hydroxide calcium isophthalate;

activated carbon-supported diethylsulfonium hydroxide calcium isophthalate;

activated carbon-supported triethylsulfonium hydroxide calcium isophthalate;

activated carbon-supported tetraethylsulfonium hydroxide calcium isophthalate;

activated carbon-supported propylsulfonium hydroxide calcium isophthalate;

activated carbon-supported dipropylsulfonium hydroxide calcium isophthalate;

activated carbon-supported tripropylsulfonium hydroxide calcium isophthalate;

activated carbon-supported tetrapropylsulfonium hydroxide calcium isophthalate;

activated carbon-supported phenylsulfonium hydroxide calcium isophthalate;

activated carbon-supported diphenylsulfonium hydroxide calcium isophthalate;

activated carbon-supported triphenylsulfonium hydroxide calcium isophthalate;

activated carbon-supported tetraphenylsulfonium hydroxide calcium isophthalate;

activated carbon-supported pyrrolium hydroxide sodium boronate;

activated carbon-supported imidazolium hydroxide sodium boronate;

activated carbon-supported pyrazolium hydroxide sodium boronate;

activated carbon-supported oxazolium hydroxide sodium boronate;

activated carbon-supported thiazolium hydroxide sodium boronate;

activated carbon-supported pyridinium hydroxide sodium boronate;

activated carbon-supported pyrimidinium hydroxide sodium boronate;

activated carbon-supported pyrazinium hydroxide sodium boronate;

activated carbon-supported pyradizimium hydroxide sodium boronate;

activated carbon-supported thiazinium hydroxide sodium boronate;

activated carbon-supported morpholinium hydroxide sodium boronate;

activated carbon-supported piperidinium hydroxide sodium boronate;

activated carbon-supported piperizinium hydroxide sodium boronate;

activated carbon-supported pyrollizinium hydroxide sodium boronate;

activated carbon-supported triphenyl phosphonium hydroxide sodium boronate;

activated carbon-supported trimethyl phosphonium hydroxide sodium boronate;

activated carbon-supported triethyl phosphonium hydroxide sodium boronate;

activated carbon-supported tripropyl phosphonium hydroxide sodium boronate;

activated carbon-supported tributyl phosphonium hydroxide sodium boronate;

activated carbon-supported trifluoro phosphonium hydroxide sodium boronate;

activated carbon-supported sulfonium hydroxide sodium boronate;

activated carbon-supported methylsulfonium hydroxide sodium boronate;

activated carbon-supported dimethylsulfonium hydroxide sodium boronate;

activated carbon-supported trimethylsulfonium hydroxide sodium boronate;

activated carbon-supported tetramethylsulfonium hydroxide sodium boronate;

activated carbon-supported ethylsulfonium hydroxide sodium boronate;

activated carbon-supported diethylsulfonium hydroxide sodium boronate;

activated carbon-supported triethylsulfonium hydroxide sodium boronate;

activated carbon-supported tetraethylsulfonium hydroxide sodium boronate;

activated carbon-supported propylsulfonium hydroxide sodium boronate;

activated carbon-supported dipropylsulfonium hydroxide sodium boronate;

activated carbon-supported tripropylsulfonium hydroxide sodium boronate;

activated carbon-supported tetrapropylsulfonium hydroxide sodium boronate;

activated carbon-supported phenylsulfonium hydroxide sodium boronate;

activated carbon-supported diphenylsulfonium hydroxide sodium boronate;

activated carbon-supported triphenylsulfonium hydroxide sodium boronate;

activated carbon-supported tetraphenylsulfonium hydroxide sodium boronate;

activated carbon-supported pyrrolium hydroxide potassium boronate;

activated carbon-supported imidazolium hydroxide potassium boronate;

activated carbon-supported pyrazolium hydroxide potassium boronate;

activated carbon-supported oxazolium hydroxide potassium boronate;

activated carbon-supported thiazolium hydroxide potassium boronate;

activated carbon-supported pyridinium hydroxide potassium boronate;

activated carbon-supported pyrimidinium hydroxide potassium boronate;

activated carbon-supported pyrazinium hydroxide potassium boronate;

activated carbon-supported pyradizimium hydroxide potassium boronate;

activated carbon-supported thiazinium hydroxide potassium boronate;

activated carbon-supported morpholinium hydroxide potassium boronate;

activated carbon-supported piperidinium hydroxide potassium boronate;

activated carbon-supported piperizinium hydroxide potassium boronate;

activated carbon-supported pyrollizinium hydroxide potassium boronate;

activated carbon-supported triphenyl phosphonium hydroxide potassium boronate;

activated carbon-supported trimethyl phosphonium hydroxide potassium boronate;

activated carbon-supported triethyl phosphonium hydroxide potassium boronate;

activated carbon-supported tripropyl phosphonium hydroxide potassium boronate;

activated carbon-supported tributyl phosphonium hydroxide potassium boronate;

activated carbon-supported trifluoro phosphonium hydroxide potassium boronate;

activated carbon-supported sulfonium hydroxide potassium boronate;

activated carbon-supported methylsulfonium hydroxide potassium boronate;

activated carbon-supported dimethylsulfonium hydroxide potassium boronate;

activated carbon-supported trimethylsulfonium hydroxide potassium boronate;

activated carbon-supported tetramethylsulfonium hydroxide potassium boronate;

activated carbon-supported ethylsulfonium hydroxide potassium boronate;

activated carbon-supported diethylsulfonium hydroxide potassium boronate;

activated carbon-supported triethylsulfonium hydroxide potassium boronate;

activated carbon-supported tetraethylsulfonium hydroxide potassium boronate;

activated carbon-supported propylsulfonium hydroxide potassium boronate;

activated carbon-supported dipropylsulfonium hydroxide potassium boronate;

activated carbon-supported tripropylsulfonium hydroxide potassium boronate;

activated carbon-supported tetrapropylsulfonium hydroxide potassium boronate;

activated carbon-supported phenylsulfonium hydroxide potassium boronate;

activated carbon-supported diphenylsulfonium hydroxide potassium boronate;

activated carbon-supported triphenylsulfonium hydroxide potassium boronate;

activated carbon-supported tetraphenylsulfonium hydroxide potassium boronate;

activated carbon-supported pyrrolium hydroxide magnesium boronate;

activated carbon-supported imidazolium hydroxide magnesium boronate;

activated carbon-supported pyrazolium hydroxide magnesium boronate;

activated carbon-supported oxazolium hydroxide magnesium boronate;

activated carbon-supported thiazolium hydroxide magnesium boronate;

activated carbon-supported pyridinium hydroxide magnesium boronate;

activated carbon-supported pyrimidinium hydroxide magnesium boronate;

activated carbon-supported pyrazinium hydroxide magnesium boronate;

activated carbon-supported pyradizimium hydroxide magnesium boronate;

activated carbon-supported thiazinium hydroxide magnesium boronate;

activated carbon-supported morpholinium hydroxide magnesium boronate;

activated carbon-supported piperidinium hydroxide magnesium boronate;

activated carbon-supported piperizinium hydroxide magnesium boronate;

activated carbon-supported pyrollizinium hydroxide magnesium boronate;

activated carbon-supported triphenyl phosphonium hydroxide magnesium boronate;

activated carbon-supported trimethyl phosphonium hydroxide magnesium boronate;

activated carbon-supported triethyl phosphonium hydroxide magnesium boronate;

activated carbon-supported tripropyl phosphonium hydroxide magnesium boronate;

activated carbon-supported tributyl phosphonium hydroxide magnesium boronate;

activated carbon-supported trifluoro phosphonium hydroxide magnesium boronate;

activated carbon-supported sulfonium hydroxide magnesium boronate;

activated carbon-supported methylsulfonium hydroxide magnesium boronate;

activated carbon-supported dimethylsulfonium hydroxide magnesium boronate;

activated carbon-supported trimethylsulfonium hydroxide magnesium boronate;

activated carbon-supported tetramethylsulfonium hydroxide magnesium boronate;

activated carbon-supported ethylsulfonium hydroxide magnesium boronate;

activated carbon-supported diethylsulfonium hydroxide magnesium boronate;

activated carbon-supported triethylsulfonium hydroxide magnesium boronate;

activated carbon-supported tetraethylsulfonium hydroxide magnesium boronate;

activated carbon-supported propylsulfonium hydroxide magnesium boronate;

activated carbon-supported dipropylsulfonium hydroxide magnesium boronate;

activated carbon-supported tripropylsulfonium hydroxide magnesium boronate;

activated carbon-supported tetrapropylsulfonium hydroxide magnesium boronate;

activated carbon-supported phenylsulfonium hydroxide magnesium boronate;

activated carbon-supported diphenylsulfonium hydroxide magnesium boronate;

activated carbon-supported triphenylsulfonium hydroxide magnesium boronate;

activated carbon-supported tetraphenylsulfonium hydroxide magnesium boronate;

activated carbon-supported pyrrolium hydroxide calcium boronate;

activated carbon-supported imidazolium hydroxide calcium boronate;

activated carbon-supported pyrazolium hydroxide calcium boronate;

activated carbon-supported oxazolium hydroxide calcium boronate;

activated carbon-supported thiazolium hydroxide calcium boronate;

activated carbon-supported pyridinium hydroxide calcium boronate;

activated carbon-supported pyrimidinium hydroxide calcium boronate;

activated carbon-supported pyrazinium hydroxide calcium boronate;

activated carbon-supported pyradizimium hydroxide calcium boronate;

activated carbon-supported thiazinium hydroxide calcium boronate;

activated carbon-supported morpholinium hydroxide calcium boronate;

activated carbon-supported piperidinium hydroxide calcium boronate;

activated carbon-supported piperizinium hydroxide calcium boronate;

activated carbon-supported pyrollizinium hydroxide calcium boronate;

activated carbon-supported triphenyl phosphonium hydroxide calcium boronate;

activated carbon-supported trimethyl phosphonium hydroxide calcium boronate;

activated carbon-supported triethyl phosphonium hydroxide calcium boronate;

activated carbon-supported tripropyl phosphonium hydroxide calcium boronate;

activated carbon-supported tributyl phosphonium hydroxide calcium boronate;

activated carbon-supported trifluoro phosphonium hydroxide calcium boronate;

activated carbon-supported sulfonium hydroxide calcium boronate;

activated carbon-supported methylsulfonium hydroxide calcium boronate;

activated carbon-supported dimethylsulfonium hydroxide calcium boronate;

activated carbon-supported trimethylsulfonium hydroxide calcium boronate;

activated carbon-supported tetramethylsulfonium hydroxide calcium boronate;

activated carbon-supported ethylsulfonium hydroxide calcium boronate;

activated carbon-supported diethylsulfonium hydroxide calcium boronate;

activated carbon-supported triethylsulfonium hydroxide calcium boronate;

activated carbon-supported tetraethylsulfonium hydroxide calcium boronate;

activated carbon-supported propylsulfonium hydroxide calcium boronate;

activated carbon-supported dipropylsulfonium hydroxide calcium boronate;

activated carbon-supported tripropylsulfonium hydroxide calcium boronate;

activated carbon-supported tetrapropylsulfonium hydroxide calcium boronate;

activated carbon-supported phenylsulfonium hydroxide calcium boronate;

activated carbon-supported diphenylsulfonium hydroxide calcium boronate;

activated carbon-supported triphenylsulfonium hydroxide calcium boronate; and

activated carbon-supported tetraphenylsulfonium hydroxide calcium boronate.

### Lignin Digestion

In another aspect, provided herein are methods for digesting lignin using the catalysts described herein. Lignin digestion may involve depolymerization, or partial depolymerization, of lignin. Lignin is contacted with any one of the catalysts described herein and one or more solvents, which forms a reaction mixture. The lignin composition in the reaction mixture is degraded to produce a liquid phase and a solid phase. The liquid phase includes one or more lignin digestion products. The solid phase includes residual lignin. The one or more lignin digestion products may be isolated from the liquid phase.

a) Lignin Composition

The lignin composition contacted with any one of the catalysts described herein may be lignocellulosic biomass, or residual undigested lignin resulting from saccharification of lignocellulosic biomass. Any suitable methods known in the art for saccharification of lignocellulosic biomass may be used to obtain a lignin composition that can be used with the catalysts described herein. For example, the lignin composition may be the undigested lignin from enzymatic or chemical hydrolysis of lignocelullosic biomass.

In some embodiments, the lignin composition used in the methods described herein may be obtained from softwood, hardwood, cassava, bagasse, oil palm, corn stover, food waste, enzymatic digestion residuals, and beer bottoms.

Softwoods (also known as conifers) may include, for example, Araucaria (e.g., Hoop Pine, Parana Pine, Chile Pine), Cedar (e.g., red cedar, white cedar, yellow cedar), Celery Top Pine, Cypress (e.g., Arizona Cypress, Bald Cypress, Hinoki Cypress, Lawson's Cypress, Mediterranean Cypress), Rocky Mountain Douglas-Fir, European Yew, Fir (e.g., Balsam Fir, Silver Fir, Noble Fir), Hemlock (e.g., Eastern Hemlock, Mountain Hemlock, Western Hemlock), Huan Pine, Kauri, Kaya, Larch (e.g., European Larch, Japanese Larch, Tamarack Larch, Western Larch), Pine (e.g., Corsican Pine, Jack Pine, Lodgepole Pine, Monterey Pine, Ponderosa Pine, Red Pine, Scots Pine, White Pine (e.g., Eastern White Pine, Western White Pine, Sugar Pine), Southern Yellow Pine (e.g., Loblolly Pine, Longleaf Pine, Pitch Pine, Shortleaf Pine), Redcedar (e.g., Eastern Redcedar, Western Redcedar), Redwood, Rimu, Spruce (e.g., Norway Spruce, Black Spruce, Red Spruce, Sitka Spruce, White Spruce), Sugi, Whitecedar (e.g., Northern Whitecedar, Southern Whitecedar), and Yellowcedar. In one embodiment, the softwood is pine.

Hardwoods (also known as angiosperms) may include, for example, African Zebrawood, Afzelia, Agba, Alder (e.g., Black Alder, Red Alder), Applewood, Ash (e.g., Black Ash, Blue Ash, Common Ash, Green Ash, Oregon Ash, Pumpkin Ash, White Ash), Aspen (e.g., Bigtooth Aspen, European Aspen, Quaking Aspen), Australian Red Cedar, Ayan, Balsa, Basswood (e.g., American Basswoord, White Basswood), Beech (e.g., European Beech, American Beech), Birch (e.g., Gray Birch, River Birch, Paper Birch, Sweet Birch, Yellow Birch, Silver Birch, White Birch), Blackbean, Blackgum, Blackwood (e.g., Australian Blackwood, African Blackwood), Bloodwood, Bocote, Boxelder, Brazilwood, Bubinga, Buckeye (e.g., Common Horse-Chestnut, Ohio Buckeye, Yellow Buckeye), Butternut, Camphor Laurel, Carapa, Catalpa, Cherry (e.g., Black Cherry, Red Cherry, Whild Cherry), Chestnut (e.g., Cape Chestnut), Coachwood, Cocobolo, Corkwood, Cottonwood (e.g., Balsam Poplar, Eastern Cottonwood, Plains Cottonwood, Swamp Cottonwood), Cucumbertree, Dogwood (e.g., Flowering Dogwood, Pacific Dogwood), Ebony (e.g., Andaman Marble-Wood, Ebène Marbre, Gabon Ebony), Elm (e.g., American Elm, English Elm, Rock Elm, Red Elm, Wych Elm), Eucalyptus (e.g., White Mahogany, Souther Mahogany, River Red Gum, Karri, Blue Gum, Flooded Gum, West Australian Eucalyptus, Tallowwood, Grey Ironbark, Blackbutt, Tasmanian Oak, Red Mahogany, Swamp Mahogany, Blue Gum, Ironbark), Goncalo Alves, Greenheart, Grenadilla, Gum, Hackberry, Hickory (e.g., Mockernut Hickory, Pecan, Pignut Hickory, Shagbark Hickory, Shellbark Hickory), Hornbeam, Hophornbeam, Ipê, Iroko, Brazilian rosewood, Jatobá, Kingwood, Lacewood, Laurel, Limba, Lignum vitae, Locust (e.g., Black Locust, Yellow Locust, Honey Locust), Maple (e.g., Sugar Maple, Black Maple, Manitoba Maple, Red Maple, Silver Maple, Sycamore Maple), Oak (e.g., Bur Oak, White Oak, Post Oak, Swamp White Oak, Southern Live Oak, Swamp Chestnut Oak, Chestnut Oak, Chinkapin Oak, Canyon Live Oak, Overcup Oak, English Oak, Red Oak, Black Oak, Laurel Oak, Southern Red Oak, Water Oak, Willow Oak, Nuttall's Oak), Obeche, Okoumé, Olive, Oregan Myrtle, California Bay Laurel, Padauk Palisander, Pear, Pink Ivory, Poplar (e.g., Balsam Poplar, Black Poplar, Hybrid Poplar, Yellow Poplar), Purple Heart, Ramin, Redheart, Teak, Walnut (e.g., Black Walnut, Persian Walnut, Brazilian Walnut), Wenge, and Willow (e.g., Black Willow, Cricket-Bat Willow, White Willow). In certain embodiments, the hardwood is selected from birch, eucalyptus, aspen, maple, and any combination thereof.

The softwood or hardwood used in the methods described herein may be in any suitable form including, for example, chips, sawdust, bark, and any combination thereof.

Cassava (Manihot esculenta) is a woody shrub of the Euphorbiaceae (spurge family). Cassava stems may be used in the methods described herein.

Bagasse is the fibrous material that remains after sugarcane or sorghum stalks are crushed from juice extraction.

Oil palm may include, for example, African Oil Palm, American Oil Palm, and Malaysian Oil Palm. The oil palm used in the methods described herein may be a palm oil waste material selected from empty fruit bunches, mesocarb fibre, palm kernel shell, and nut. In one embodiment, the oil palm is empty fruit bunch or mesocarb fibre.

Corn stover includes the leaves and stalks of maize (Zea mays).

Food waste may include any food substance, in solid and/or liquid form, that is raw or cooked that is discarded or intends to be discarded. Food waste includes organic residues generated by the handling, storage, sale, preparation, cooking and serving of foods.

Enzymatic digestion residuals may include any residual biomass materials, in solid and/or liquid form, that results from the enzymatic hydrolysis of biomass. Enzymatic digestion residuals may include residual amounts of cellulose, hemicellulose, and/or lignin.

Beer bottoms may include any residual materials that results from the fermentation in a beer brewing process.

In some embodiments, the solid base catalysts described herein may be used with a lignin composition that has been pretreated. Pretreatment methods for the lignin component of lignocellulosic biomass are known to one skilled in the art and may include, for example, incomplete pulping, the Kraft process, solvent extraction, treatment with acetic acid, the organosolv process. See, e.g., Biermann, Christopher J., “Handbook of Pulping and Papermaking, Second Edition” San Diego, Academic Press, 1996.

b) Lignin Digestion Conditions

The digestion of lignin using the catalysts described herein may be performed in stirred-tank reactors or vessels under controlled pH, temperature, and mixing conditions. One skilled in the art would recognize that suitable processing time, temperature and pH conditions may vary depending on the solid base catalyst and solvents used. These factors are described in further detail below.

Processing Time, Temperature and pH Conditions

In some embodiments, the digestion of lignin can last up to 200 hours. In other embodiments, the digestion of lignin can take place from 1 to 96 hours, from 12 to 72 hours, or from 12 to 48 hours.

In some embodiments, the digestion of lignin is performed at temperature in the range of about 25° C. to about 150° C. In other embodiments, the digestion of lignin is performed at temperature in the range of about 30° C. to about 125° C., or about 80° C. to about 120° C., or about 100° C. to 110° C.

The pH for the digestion of lignin is generally affected by the intrinsic properties of the solid base catalyst used. In particular, the basic moiety of the solid base catalyst may affect the pH of the digestion of lignin. For example, in one embodiment, the use of imidazolium hydroxide moiety in a solid base catalyst results in the digestion of lignin at a pH above 7. In other embodiments, the digestion of lignin may be performed at a pH between 7 and 11. In certain embodiments, the reacted effluent may have a pH of at least 7.0. It should be understood that the pH may be compatible with other processes such as hydrogenation, distillation, chemical or biological oxidation, and precipitation. It should also be understood, however, that the pH can be modified and controlled by the addition of acids, bases or buffers. Moreover, the pH may vary within the reactor.

In certain embodiments, the digestion of lignin methods described herein may further include monitoring the pH of the digestion of lignin reaction, and optionally adjusting the pH within the reactor. In some instances, as a high pH in solution may indicate an unstable solid base catalyst, in which the catalyst may be losing at least a portion of its basic groups to the surrounding environment through leaching. In some embodiments, the pH near the surface of the solid base catalyst is above about 12.

Amount of Feedstock Used

The amount of the feedstock used in the methods described herein relative to the amount solvent used may affect the rate of reaction and yield. The amount of the lignin composition used may be characterized by the dry solids content. In certain embodiments, dry solids content refers to the total solids of a slurry as a percentage on a dry weight basis. In some embodiments, the dry solids content of the lignin composition is between about 5 wt % to about 95 wt %, between about 10 wt % to about 80 wt %, between about 15 to about 75 wt %, or between about 15 to about 50 wt %.

Amount of Solid Base Catalyst Used

The amount of the solid base catalysts used in the methods for digesting lignin described herein may depend on several factors including, for example, the concentration of lignin in the lignin composition, the type and number of pretreatment(s) applied to the lignin composition, and the reaction conditions (e.g., temperature, time, and pH). In one embodiment, the weight ratio of the solid base catalyst to the lignin composition is about 0.1 g/g to about 50 g/g, about 0.1 g/g to about 25 g/g, about 0.1 g/g to about 10 g/g, about 0.1 g/g to about 5 g/g, about 0.1 g/g to about 2 g/g, about 0.1 g/g to about 1 g/g, or about 0.1 to about 1.0 g/g.

Solvent

In certain embodiments, the digestion of lignin using the solid base catalyst described herein may be carried out in one or more solvents. The digestion may be carried out in an aqueous environment. In one embodiment, the one or more solvents include water, which may be obtained from various sources. Generally, water sources with lower concentrations of ionic species are preferable, as such ionic species may reduce effectiveness of the solid base catalyst. In some embodiments where the aqueous solvent includes water, the water has less than 10% of ionic species (e.g., salts of sodium, phosphorous, ammonium, magnesium, or other species found naturally in lignocellulosic biomass).

In certain embodiments, the methods for digesting lignin described herein may further include monitoring the amount of water present in the reaction and/or the ratio of water to the lignin composition over a period of time. In other embodiments, the methods for digesting lignin described herein may further include supplying water directly to the reaction, for example, in the form of steam or steam condensate. For example, in some embodiments, the hydration conditions in the reactor is such that the water-to-lignin ratio is 5:1, 4:1, 3:1, 2:1, 1:1, 1:2, 1:3, 1:4, or 1:5, or less than 1:5. It should be understood, however, that the ratio of water to lignin may be adjusted based on the specific solid base catalyst used.

In other embodiments, the methods for digesting lignin described herein may further include the use of a mixture of solvents. In some embodiments, the solvents in the mixture may be water-miscible. In other embodiments, the solvents in the mixture are water-immiscible. In certain embodiments, the solvents in the mixture may be selected from water, acetone, ethanol, propanol, butanol, dimethylsulfoxide, dimethylformamide, toluene, benzene, ethyl acetate, acetic acid, formic acid, tri-alkyl amines, alkanes, and biodiesel.

Batch Versus Continuous Processing

Generally, the solid base catalyst and the feedstock are introduced into an interior chamber of a reactor, either concurrently or sequentially. The digestion of lignin can be performed in a batch process or a continuous process. For example, in one embodiment, the digestion of lignin is performed in a batch process, where the contents of the reactor are continuously mixed or blended, and all or a substantial amount of the products of the reaction are removed. In one variation, the digestion of lignin is performed in a batch process, where the contents of the reactor are initially intermingled or mixed but no further physical mixing is performed. In another variation, the digestion of lignin is performed in a batch process, wherein once further mixing of the contents, or periodic mixing of the contents of the reactor, is performed (e.g., at one or more times per hour), all or a substantial amount of the products of the reaction are removed after a certain period of time.

In other embodiments, the digestion of lignin is performed in a continuous process, where the contents flow through the reactor with an average continuous flow rate but with no explicit mixing. After introduction of the solid base catalyst and the feedstock into the reactor, the contents of the reactor are continuously or periodically mixed or blended, and after a period of time, less than all of the products of the reaction are removed. In one variation, the digestion of lignin is performed in a continuous process, where the mixture containing the catalyst and feedstock is not actively mixed. Additionally, mixing of catalyst and feedstock may occur as a result of the redistribution of solid base catalysts settling by gravity, or the non-active mixing that occurs as the material flows through a continuous reactor.

Reactors

The reactors used for the methods for digesting lignin described herein may be open or closed reactors suitable for use in containing the chemical reactions described herein. Suitable reactors may include, for example, a fed-batch stirred reactor, a batch stirred reactor, a continuous flow stirred reactor with ultrafiltration, a continuous plug-flow column reactor, an attrition reactor, or a reactor with intensive stirring induced by an electromagnetic field. See e.g., Fernanda de Castilhos Corazza, Flavio Faria de Moraes, Gisella Maria Zanin and Ivo Neitzel, Optimal control in fed-batch reactor for the cellobiose hydrolysis, Acta Scientiarum. Technology, 25: 33-38 (2003); Gusakov, A. V., and Sinitsyn, A. P., Kinetics of the enzymatic hydrolysis of cellulose: 1. A mathematical model for a batch reactor process, Enz. Microb. Technol., 7: 346-352 (1985); Ryu, S. K., and Lee, J. M., Bioconversion of waste cellulose by using an attrition bioreactor, Biotechnol. Bioeng. 25: 53-65(1983); Gusakov, A. V., Sinitsyn, A. P., Davydkin, I. Y., Davydkin, V. Y., Protas, O. V., Enhancement of enzymatic cellulose hydrolysis using a novel type of bioreactor with intensive stirring induced by electromagnetic field, Appl. Biochem. Biotechnol., 56: 141-153(1996). Other suitable reactor types may include, for example, fluidized bed, upflow blanket, immobilized, and extruder type reactors for hydrolysis and/or fermentation.

In certain embodiments where the digestion of lignin is performed as a continuous process, the reactor may include a continuous mixer, such as a screw mixer. The reactors may be generally fabricated from materials that are capable of withstanding the physical and chemical forces exerted during the processes described herein. In some embodiments, such materials used for the reactor are capable of tolerating high concentrations of strong liquid bases; however, in other embodiments, such materials may not be resistant to strong bases.

Further, the reactor typically contains an outlet means for removal of contents (e.g., a sugar-containing solution) from the reactor. Optionally, such outlet means is connected to a device capable of processing the contents removed from the reactor. Alternatively, the removed contents are stored. In some embodiments, the outlet means of the reactor is linked to a continuous incubator into which the reacted contents are introduced. The reactor may be filled with biomass by a top-load feeder containing a hopper capable of holding biomass. Further, the outlet means provides for removal of residual biomass by, e.g., a screw feeder, by gravity, or a low shear screw.

It should also be understood that additional feedstock and/or catalyst may be added to the reactor, either at the same time or one after the other.

Rate and Yield of Lignin Digestion

The digestion of lignin using the solid base catalysts described herein may produce one or more lignin digestion products such as monolignols, phenylpropenes, monolignolglucosides, and any combinations thereof. In certain embodiments, the one or more lignin digestion products may include, for example, p-coumaryl alcohol, coumarilin, coniferyl alchol, coniferin, sinapyl alcohol, sinaplin, eugenol, chavicol, safrole, estragol, and any combinations thereof.

The use of the solid base catalysts described herein can increase the rate and/or yield of lignin digestion. The ability of the solid base catalyst to break down lignin into one or more lignin digestion products can be measured by determining the effective first-order rate constant,

$${k}_{1} ( species i ) = - \frac{ln ( 1 - {X}_{i} )}{Δ t} ,$$

where Δt is the duration of the reaction and Xᵢ is the extent of reaction for species i. In some embodiments, the solid base catalysts described herein are capable of breaking down lignin into one or more lignin digestion products at a first-order rate constant of at least 0.001 per hour, at least 0.01 per hour, at least 0.1 per hour, at least 0.2 per hour, at least 0.3 per hour, at least 0.4 per hour, at least 0.5 per hour, or at least 0.6 per hour.

d) Separation and Purification of the Lignin Digestion Products

In some embodiments, the method for producing one or more lignin digestion products from the feedstock using the solid base catalysts described herein further includes recovering the one or more lignin digestion products.

The lignin digestion products, which are typically soluble in the one or more solvents used to the reaction to digest lignin, can be separated from the insoluble residual feedstock using technology well known in the art such as, for example, centrifugation, filtration, gravity settling, distillation, and solvent extraction.

Separation of the lignin digestion products may be performed in the reactor or in a separator vessel. In an exemplary embodiment, the method for producing one or more lignin digestion products is performed in a system with a reactor and a separator vessel. Reactor effluent containing the lignin digestion products is transferred into a separator vessel and is washed with a solvent (e.g., water), by adding the solvent into the separator vessel and then separating the solvent in a continuous centrifuge. Alternatively, in another exemplary embodiment, a reactor effluent containing residual solids (e.g., residual undigested lignin) is removed from the reactor vessel and washed, for example, by conveying the solids on a porous base (e.g., a mesh belt) through a solvent (e.g., water) wash stream. Following contact of the stream with the reacted solids, a liquid phase containing the lignin digestion products is generated. Optionally, residual solids may be separated by a cyclone. Suitable types of cyclones used for the separation may include, for example, tangential cyclones, spark and rotary separators, and axial and multi-cyclone units.

In another embodiment, separation of the lignin digestion products is performed by batch or continuous differential sedimentation. Reactor effluent is transferred to a separation vessel, optionally combined with water and/or enzymes for further treatment of the effluent. Over a period of time, solid biomaterials (e.g., residual treated feedstock), the solid base catalyst, and the lignin digestion products-containing aqueous material can be separated by differential sedimentation into a plurality of phases (or layers). Generally, the catalyst layer may sediment to the bottom, and depending on the density of the residual feedstock, the feedstock phase may be on top of, or below, the aqueous phase. When the phase separation is performed in a batch mode, the phases are sequentially removed, either from the top of the vessel or an outlet at the bottom of the vessel. When the phase separation is performed in a continuous mode, the separation vessel contains one or more than one outlet means (e.g., two, three, four, or more than four), generally located at different vertical planes on a lateral wall of the separation vessel, such that one, two, or three phases are removed from the vessel. The removed phases are transferred to subsequent vessels or other storage means. By these processes, one of skill in the art would be able to capture (1) the catalyst layer and the aqueous layer or biomass layer separately, or (2) the catalyst, aqueous, and biomass layers separately, allowing efficient catalyst recycling, retreatment of undigested feedstock, and separation of lignin digestion products. Moreover, controlling rate of phase removal and other parameters allows for increased efficiency of catalyst recovery. Subsequent to removal of each of the separated phases, the catalyst and/or feedstock may be separately washed by the aqueous layer to remove adhered lignin digestion products.

The lignin digestion products isolated from the vessel may be subjected to further processing steps (e.g., as drying, fermentation) to produce bio-based products. In some embodiments, the lignin digestion products that are isolated may be at least 1% pure, at least 5% pure, at least 10% pure, at least 20% pure, at least 40% pure, at least 60% pure, at least 80% pure, at least 90% pure, at least 95% pure, at least 99% pure, or greater than 99% pure, as determined by analytical procedures known in the art, such as determination by high performance liquid chromatography (HPLC), functionalization and analysis by gas chromatography, mass spectrometry, spectrophotometric procedures based on chromophore complexation and/or carbohydrate oxidation-reduction chemistry.

The residual undigested feedstock isolated from the vessels may be useful as a combustion fuel or as a feed source of non-human animals such as livestock.

e) Recovery of the Solid Base Catalysts

The solid base catalysts may be recovered and reused. Sedimentation of the solid base catalyst is used to recover the catalyst following use. In particular, the catalyst may sink, while other residuals solids may remain suspended in the saccharification reaction mixture.

Sedimentation rate can be measured by the sedimentation coefficient,

$$s = \frac{mv}{F}$$

where m is the mass of the particle, v is its sinking velocity (terminal velocity of the sinking particle in the selected solvent), and F is the force applied to cause the sinking. For gravity sedimentation, F=mg, and

$$s = \frac{v}{g}$$

where g is the acceleration due to gravity.

For simple gravimetric sedimentation in water, the sedimentation rate of the solid base catalyst may, in some embodiments, be 10⁻⁶-10⁻², 10⁻⁵-10⁻³, or 10⁴-10⁻³.

The density of the solid base catalyst may also have an impact on its ease of recovery. In some embodiments, the gravimetric density of the solid base catalyst is 0.5-3.0 kg/L, 1.0-3.0 kg/L, or 1.1-3.0 kg/L. One of skill in the art would recognize that various methods and techniques suitable for measuring the density of a solid catalyst.

### Catalyst-Containing Compositions

Provided herein are also compositions involving the solid base catalysts that can be used in a variety of methods described herein, including the digestion or break down of lignin.

Provided are compositions that include lignin and the solid base catalysts described herein. In some embodiments, the composition further includes one or more solvents. In one embodiment, the one or more solvents is one solvent, wherein the solvent is water. In other embodiments, the one or more solvents are selected from water, acetone, ethanol, propanol, butanol, dimethylsulfoxide, dimethylformamide, toluene, bezene, ethyl acetate, acetic acid, formic acid, tri-alkyl amines, alkanes, and biodiesel.

Provided are also compositions that include the solid base catalysts described herein, one or more lignin digestion products, and residual lignin. In some embodiments, the one or more lignin digestion products are selected from monolignols, phenylpropenes, monolignolglucosides, and any combinations thereof. In certain embodiments, the one or more lignin digestion products include p-coumaryl alcohol, coumarilin, coniferyl alchol, coniferin, sinapyl alcohol, sinaplin, eugenol, chavicol, safrole, estragol, and any combinations thereof.

### Catalytic Intermediates

When the solid base catalysts are used to degrade cellulosic materials, as described above, a catalytic intermediate is formed. Provided herein are also the catalytic intermediates, where the solid base catalyst coordinates with the lignin. Without wishing to be bound by any theory, the solid base catalyst may be chemically interfaced with the lignin through, for example, hydrogen-bonding pi-pi stacking, or van der waals adhesion.

General Methods of Preparing the Polymeric catalysts

a) Methods of Preparing the Polymeric Catalysts

The polymeric catalysts described herein can be made using polymerization techniques known in the art, including for example techniques to initiate polymerization of a plurality of monomer units.

For example, a polymeric catalyst can first be prepared, followed by washing the acid catalyst with a base to produce a polymeric catalyst. To prepare the polymeric catalyst, an intermediate polymer functionalized with the ionic group, but is free or substantially free of the acidic group is formed. The intermediate polymer can then be functionalized with the acidic group. In other embodiments, an intermediate polymer functionalized with the acidic group, but is free or substantially free of the ionic group. The intermediate polymer can then be functionalized with the ionic group. In yet other embodiments, the polymeric catalysts described herein can be formed by polymerizing monomers with both acidic and ionic groups. Once a polymeric acidic-ionic catalyst is produced, a polymeric catalyst can be prepared by washing the acid catalyst with a base.

In some embodiments, the polymeric catalysts described herein can be formed by first forming a first intermediate polymer, which is functionalized with a cationic group, but is free or substantially free of the basic group. The first intermediate polymer can then be functionalized with an acidic group, forming a second intermediate polymer. The second intermediate polymer can then be subjected to an ion-exchange step to convert the cationic group to the base group and the acidic group to the anionic group.

In other embodiments, the polymeric catalysts described herein can be formed by first forming a first intermediate polymer, which is functionalized with an acidic group, but is free or substantially free of the ionic group. The first intermediate polymer can then be functionalized with a cationic group, forming a second intermediate polymer. The second intermediate polymer can then be subjected to an ion-exchange step to convert the cationic group to the base group and the acidic group to the anionic group.

In yet other embodiments, the polymeric catalysts described herein can be formed by polymerizing monomers with both basic and anionic groups.

In yet other embodiments, the polymeric catalysts described herein can be formed by polymerizing monomers containing basic groups with monomers containing anionic groups.

Provided is also a method of preparing any of the polymers described herein, by:

a) providing a starting polymer;

b) combining the starting polymer with a nitrogen-containing compound or phosphorous-containing compound to produce an ionic polymer having at least one cationic group;

c) combining the ionic polymer with an effective acidifying reagent to produce an intermediate polymer; and
d) contacting the intermediate polymer with an effective amount of one or more ionic salt solutions to produce the polymer.
   It should be understood, however, that the steps described above may be performed in other orders. In other embodiments, the steps described above may be performed in the order of a), c), d), and b); or a), c), b), and d).

In some embodiments, the starting polymer is selected from polyethylene, polypropylene, polyvinyl alcohol, polycarbonate, polystyrene, polyurethane, or a combination thereof. In certain embodiments, the starting polymer is a polystyrene. In certain embodiments, the starting polymer is poly(styrene-co-vinylbenzylhalide-co-divinylbenzene). In another embodiment, the starting polymer is poly(styrene-co-vinylbenzylchloride-co-divinylbenzene).

In some embodiments of the method to prepare any of the polymers described herein, the nitrogen-containing compound is selected from a pyrrolium compound, an imidazolium compound, a pyrazolium compound, an oxazolium compound, a thiazolium compound, a pyridinium compound, a pyrimidinium compound, a pyrazinium compound, a pyradizimium compound, a thiazinium compound, a morpholinium compound, a piperidinium compound, a piperizinium compound, and a pyrollizinium compound. In certain embodiments, the nitrogen-containing compound is an imidazolium compound.

In some embodiments of the method to prepare any of the polymers described herein, the phosporus-containing compound is selected from a triphenyl phosphonium compound, a trimethyl phosphonium compound, a triethyl phosphonium compound, a tripropyl phosphonium compound, a tributyl phosphonium compound, a trichloro phosphonium compound, and a trifluoro phosphonium compound.

In some embodiments of the method to prepare any of the polymers described herein, the acid is selected from sulfuric acid, phosphoric acid, hydrochloric acid, acetic acid and boronic acid. In one embodiment, the acid is sulfuric acid.

In some embodiments, the ionic salt is a water soluble hydroxide or a slightly water soluble hydroxide. In certain embodiments, the ionic salt is a hydroxide salt of a Group 1 element. In particular embodiments, the ionic salt is sodium hydroxide, potassium hydroxide, methyl-butylimidazolium hydroxide, dimethylmorpholinium hydroxide, lithium hydroxide, cesium hydroxide, rubidium hydroxide, strontium hydroxide, barium hydroxide, or calcium hydroxide.

Also provided is a method of preparing any of the polymers described herein having a polystyrene backbone, by:

a) providing a polystyrene;

b) reacting the polystyrene with a nitrogen-containing compound to produce an ionic polymer;

c) reacting the ionic polymer with an acid to produce a third polymer; and contacting the third polymer with a salt solution to form a fourth polymer.

In certain embodiments, the polystyrene is poly(styrene-co-vinylbenzylhalide-co-divinylbenzene). In one embodiment, the polystyrene is poly(styrene-co-vinylbenzylchloride-co-divinylbenzene).

In some embodiments, the polymer has one or more catalytic properties selected from:

a) disruption of at least one hydrogen bond in cellulosic materials;

b) intercalation of the polymer into crystalline domains of cellulosic materials;

c) cleavage of at least one glycosidic bond in cellulosic materials; and

d) disruption of at least ether linkage of lignin.

Provided herein are also such intermediate polymeric catalysts, including those obtained at different points within a synthetic pathway for producing the fully functionalized polymeric catalysts described herein. In some embodiments, the polymeric catalysts described herein can be made, for example, on a scale of at least 100 g, or at least 1 kg, in a batch or continuous process.

b) Methods of Preparing the Solid-Supported Catalysts

The solid-supported catalysts described herein with carbon supports may be prepared by subjecting a carbonaceous material to: (1) support preparation, (2) support activation, and (3) support functionalization. An exemplary preparation sequence is provided in Table 1. One of skill in the art would recognize that two or more of the support preparation, support activation, and catalyst functionalization steps may be combined into a single step.

TABLE 1
Exemplary steps for preparing a dual-functionalized solid carbon supported catalyst

| Step | Reactant | Reaction | Product |
|---|---|---|---|
| 1. Support Preparation | Carbonaceous material | Partial | Carbon Support |
| | | carbonization | |
| 2. Linker Attachment | Carbon Support | Haloalkylation, | Carbon Support with |
| to Support | | haloacylation, or | Linker |
| | | diazonium | |
| | | displacement | |
| 3. First | Activated Support | Quaternization | First Functionalized |
| Functionalization | | | Support |
| 4. Second | First Functionalized | Acidification | Dual-Functionalized |
| Functionalization | Support | | Solid Carbon |
| | | | Supported Catalyst |

Support Preparation

Support preparation may be accomplished by any methods known in the art. For example, pyrolysis may be used to convert a carbonaceous material into a carbon support. Incomplete carbonization may also be employed to obtain a carbon support. In some embodiments, a carbonaceous material may be subjected to an oxygen-deficient atmosphere at a controlled temperature to produce a carbon support. In yet other embodiments, commercially-available carbon supports may be used.

The carbonaceous material may be naturally-occurring. Suitable carbonaceous materials may include, for example, shrimp shell, chitin, coconut shell, wood pulp, paper pulp, cotton, cellulose, hard wood, soft wood, wheat straw, sugarcane bagasse, cassava stem, corn stover, oil palm residue, bitumen, asphaltum, tar, coal, pitch, or any combinations thereof.

In some embodiments, the carbon content of the carbonaceous material is greater than 20% g carbon/g dry material, greater than 30% g carbon/g dry material, or greater than 40% g carbon/g dry material. In addition to carbon, the carbonaceous material may also contain oxygen, nitrogen, or a combination thereof. For example, with reference to FIG. 9A, carbon support 902 may have one or more functional groups, including for example hydroxyl, amino and carboxyl groups. In some embodiments, the oxygen content of the carbonaceous material is between 10% to 60% g oxygen/g dry material, between 20% to 40% g oxygen/g dry material, or between 20% to 30% g oxygen/g dry material. In other embodiments, the nitrogen content of the carbonaceous material is greater than 1% g nitrogen/g dry material, greater than 5% g nitrogen/g dry material, or greater than 10% g nitrogen/g dry material.

One of skill in the art would recognize that the conditions under which the carbonaceous material is carbonized may vary depending on the carbonaceous material used. In some embodiments, the carbonaceous material is carbonized in an atmosphere containing less than 20% oxygen, less than 10% oxygen, less than 1% oxygen, less than 1 part per thousand of oxygen, less than 100 parts per million of oxygen, or less than 10 parts per million of oxygen. In some embodiments, the carbonaceous material is carbonized in an atmosphere containing nitrogen. In other embodiments, the carbonaceous material is carbonized in an atmosphere containing purified nitrogen.

In some embodiments, the carbonaceous material is carbonized at a temperature between 200° C. and 500° C., between 250° C. and 400° C., or between 275° C. and 350° C. The temperature may be controlled to within plus or minus 50° C., within plus or minus 10° C., within plus or minus 5° C., or to within plus or minus 2° C. In some embodiments, the carbonaceous material is carbonized within 2 to 10 hours, within 2 to 5 hours, within 3 to 5 hours, or within 3 to 4 hours.

The carbonaceous material may undergo incomplete carbonization based on the carbonization conditions described above. Incomplete carbonization transforms the carbonaceous material into a poly-aromatic heterocyclic superstructure. The superstructure may include, for example, poly-condensed fused ring sub-structures that are attached to one another with random orientation to form the overall superstructure.

Heteroatoms, particularly oxygen and nitrogen present in the carbonaceous starting material, become incorporated into the superstructure. Some of the heteroatoms may be incorporated into the carbon support, as saturated, unsaturated, and aromatic heterocycles, many of which may be fused rings. For example, the carbon support (and hence the final solid-supported catalyst) may have furanic rings with 4-7 oxygen atoms and/or 4-7 nitrogen atoms. Some of the heteroatoms in the solid-supported catalyst may also be from the moieties attached to the carbon support. For example, oxygen may be from alcohol moieties (e.g., phenol, alcohols) and carboxylic acid moieties (e.g., formic, formyl, acetic, acetyl) covalently bonded to the edge of the heterocyclic sub-structures. Nitrogen may be from amino moieties (e.g., aniline, alkylamino).

The heteroatom content of the carbon support may affect the reactivity in functionalizing the support with acidic and/or ionic moieties. For example, the heteroatoms incorporated into the superstructure may affect the electronic nature of the carbon support, and hence its reactivity with the functional moieties.

The carbonaceous materials that may be used to prepare the carbon support may, in some embodiments, contain: 30%-70% g carbon/g starting material; 2%-8% g hydrogen/g starting material; 0%-60% g oxygen/g starting material; and 0%-60% g oxygen/g starting material. Following incomplete carbonization, the heteroatom content of the carbon support, may in some embodiments, contain: 0-40%, 5-30%, 10-30%, or 15-30% g oxygen/g backbone; and 0-15%, 2-10%, or 5-10% g nitrogen/g backbone.

The overall heteroatom content of the solid-supported catalyst may vary, depending in part on the functional moieties attached to the solid support. For example, haloacylation or haloalkylation may introduce the oxygen and/or halogen content. Quaternization (alkylation) may introduce the phosphorous and/or nitrogen content. Sulfonation may increase the sulfur and oxygen content.

In some embodiments, the solid-supported catalyst may contain: 10-50%, 15-40%, 10-30% g oxygen/g catalyst; 0-15%, 2-10%, 5-10% g nitrogen/g catalyst; 5-20%, 5-15%, or 10-15% g sulfur/g catalyst; and 5-20%, 5-15%, 8-15% g phosphorous/g catalyst.

The carbon supports prepared according to the methods described above may be used in combination with other solid supports, including for example silica, silica gel, alumina, magnesia, titania, zirconia, clays, magnesium silicate, silicon carbide, zeolites, and ceramics.

Support Activation

Support activation step involves subjecting the carbon support to a chemical functionalization reaction to attach reactive linkers to the carbon support. Suitable reactive linkers may include, for example, haloalkanes, haloacyl compounds, amines, and diazo compounds. Such reactive linkers activate the carbon support, making the support more susceptible to further functionalization to attach acidic, ionic, acidic-ionic and/or hydrophobic moieties.

In some embodiments, the reactive linker may be introduced to the carbon support by a halomethylating agent. In certain embodiments, the reactive linker may be introduced to the carbon support by a chloromethylating agent. With reference to FIG. 9A, the chloromethylating agent is chloromethyl methyl ether.

In other embodiments, the reactive linker may be introduced to the carbon support by a haloacylating agent. In certain embodiments, the reactive linker may be introduced to the carbon support by a chloroacylating agent. A suitable example of a chloroacylating agent is chloroacetyl chloride.

The chloromethylating agent or the chloroacylating agent may be enacted using a Lewis acid catalyst. In certain embodiments, the Lewis acid catalyst is selected from zinc (II) chloride, aluminum (III) chloride, and iron (III) chloride. With reference to FIG. 9A, the Lewis acid may be zinc chloride (ZnCl₂) or aluminum chloride (AlCl₃).

The reactive linker may be introduced to the carbon support via a Friedel-Crafts alkylation or a Friedel-Crafts acylation reaction. An exemplary reaction to introduce such a reactive linker to the carbon support is depicted in FIG. 9A. In some embodiments, the chloromethylating or chloroacylating reaction may be performed in an inert solvent. Suitable inert solvents may include any solvent that is suitable for a Friedel-Crafts reaction. For example, suitable inert solvents may include, for example, dichloromethane (DCM), dichloroethane (DCE), diethyl ether, tetrahydrofuran (THF), or ionic liquids.

The chloromethylation or chloroacylation reaction may be performed at a temperature below 25° C., below 10° C., below 5° C., or at or below 0° C.

With reference again to FIG. 9A, activated carbon support 904 has a chloromethane moiety as the reactive linker. It should be understood that, in other exemplary embodiments, other halo moieties may be added as a reactive linker, and a plurality of reactive linkers may be attached to the activated carbon support.

Support Functionalization

The activated solid supports may undergo one or more reactions to attach acidic and/or ionic moieties to the solid support. With reference to FIG. 9B, activated carbon support 904 is first quaternized to attach a nitrogen-containing cationic group to the solid support. The exemplary nitrogen-containing cationic group in FIG. 9B has a formula NR¹R²R³, wherein each R¹, R² and R³ is independently hydrogen or alkyl, or R¹ is taken together with R² and the nitrogen atom to which they are attached to form a heterocycloalkyl, or R¹, R² and R³ are taken together with the nitrogen atom to which they are attached to form a heteroaryl.

Quaternized solid support 906 undergoes acid-treatment to produce dual-functionalized solid supported catalyst 908. While only one cationic group and one acidic group is depicted in catalyst 908 of FIG. 9B, it should be understood that a plurality of cationic groups and a plurality of acidic groups may be attached to the solid support using the methods described herein.

In other embodiments, the activated solid support may be acidified before quaterinzation to produce a dual-functionalized solid-supported catalyst. In yet other embodiments, the activated support may be functionalized with an acidic-ionic group. In yet other embodiments, one or more other functional groups may be attached to the solid-supported catalysts, including hydrophobic groups.

Preparation of Base Catalysts

Once the catalyst is functionalized with acidic and ionic groups, a solid-supported catalyst can be prepared by washing the acid catalyst with a base. With reference again to FIG. 9B, in an exemplary process, catalyst 908 can be treated with an aqueous base (such as aqueous sodium hydroxide) to produce the solid-supported catalyst 910 with both basic and ionic moieties.

### Enumerated Embodiments

The following enumerated embodiments are representative of some aspects of the invention.

1. A catalyst comprising basic monomers and ionic monomers connected to form a polymeric backbone,

wherein each basic monomer independently comprises at least one Bronsted-Lowry base, wherein each Bronsted-Lowry base independently comprises at least one nitrogen-containing cationic group, at least one phosphorous-containing cationic group, at least one sulfur-containing cationic group, or any combinations thereof.

2. The catalyst of embodiment 1, wherein each Bronsted-Lowry base is selected from the group consisting of pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.
3. The catalyst of embodiment 1 or 2, wherein one or more of the Bronsted-Lowry bases are directly connected to the polymeric backbone.
4. The catalyst of any one of embodiments 1 to 3, wherein one or more of the basic monomers each further comprise a linker connecting the Bronsted-Lowry base to the polymeric backbone.
5. The catalyst of embodiment 4, wherein wherein each linker is independently selected from the group consisting of unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate.
6. The catalyst of embodiment 4, wherein at least one Bronsted-Lowry base and a linker form a side chain, wherein each side chain is independently selected from the group consisting of:

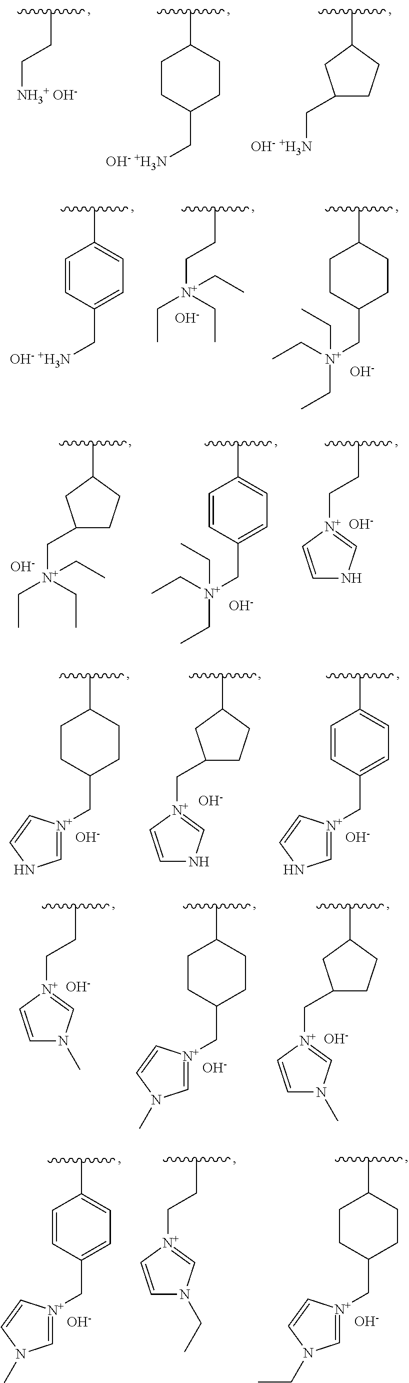

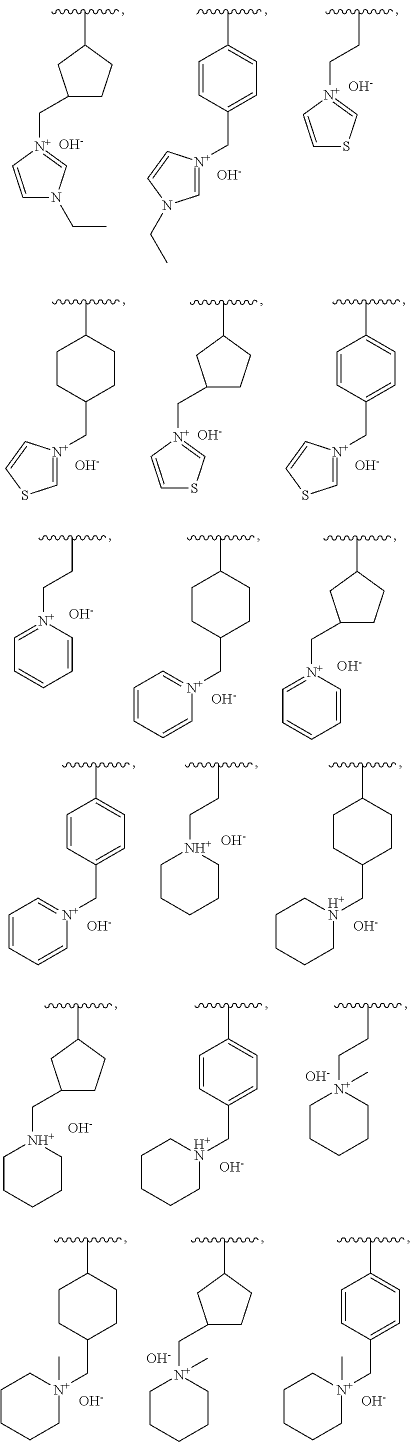

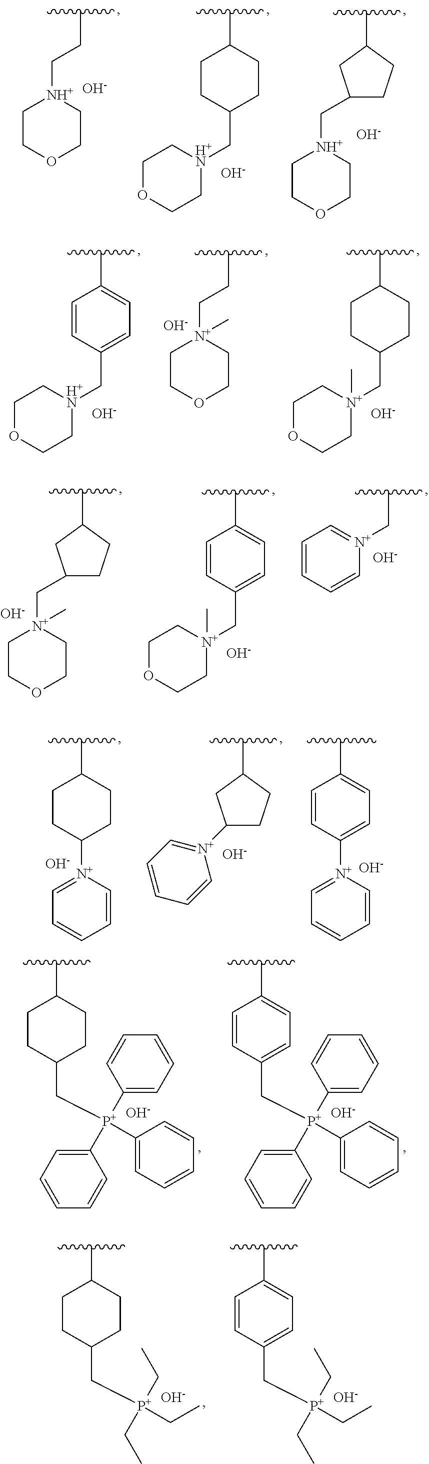

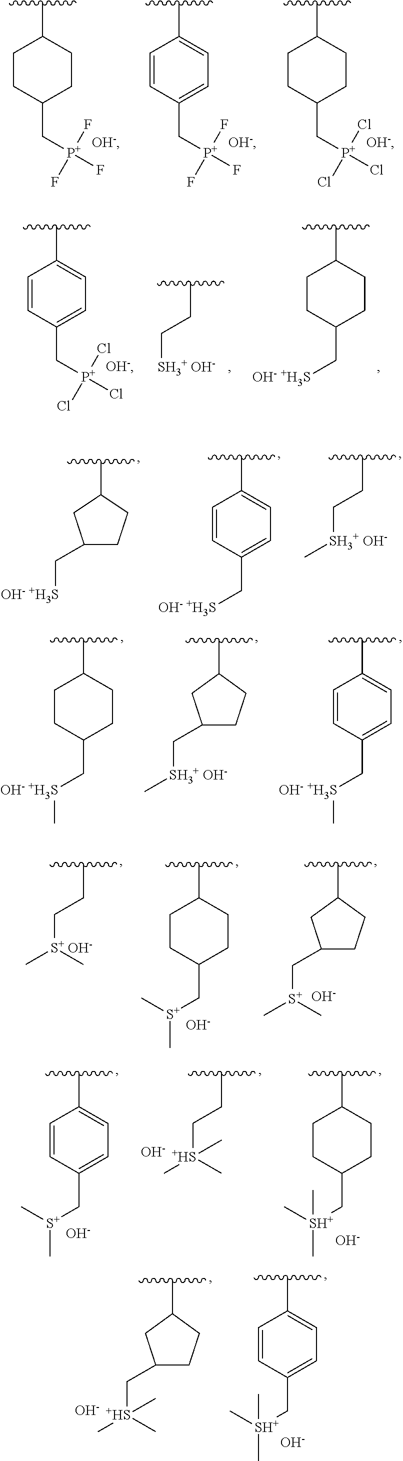

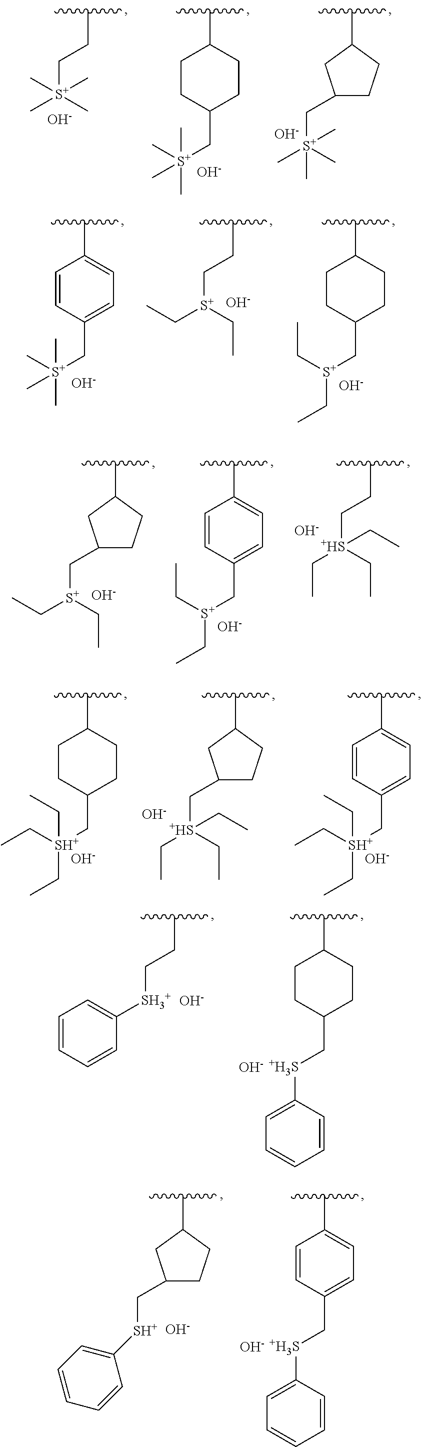

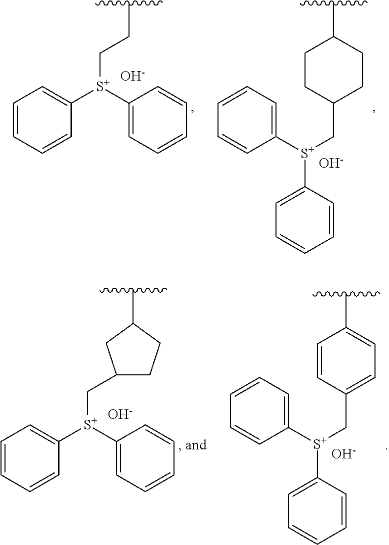

7. The catalyst of any one of embodiments 1 to 6, wherein each ionic monomer comprises at least one anionic group, wherein each anionic group is independently selected from the group consisting of sulfonate, phosphonate, acetate, isophthalate, and boronate.
8. The catalyst of any one of embodiments 1 to 7, wherein each ionic monomer comprises at least one anionic group, wherein one or more of the anionic groups are directly connected to the polymeric backbone.
9. The catalyst of any one of embodiments 1 to 7, wherein each ionic monomer comprises at least one anionic group, wherein one or more of the anionic groups are connected to the polymeric backbone by a linker.
10. The catalyst of embodiment 9, wherein each linker is independently selected from the group consisting of unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate.
11. The catalyst of embodiment 9, wherein at least one anionic group and a linker form a side chain, and wherein each side chain is selected from the group consisting of:

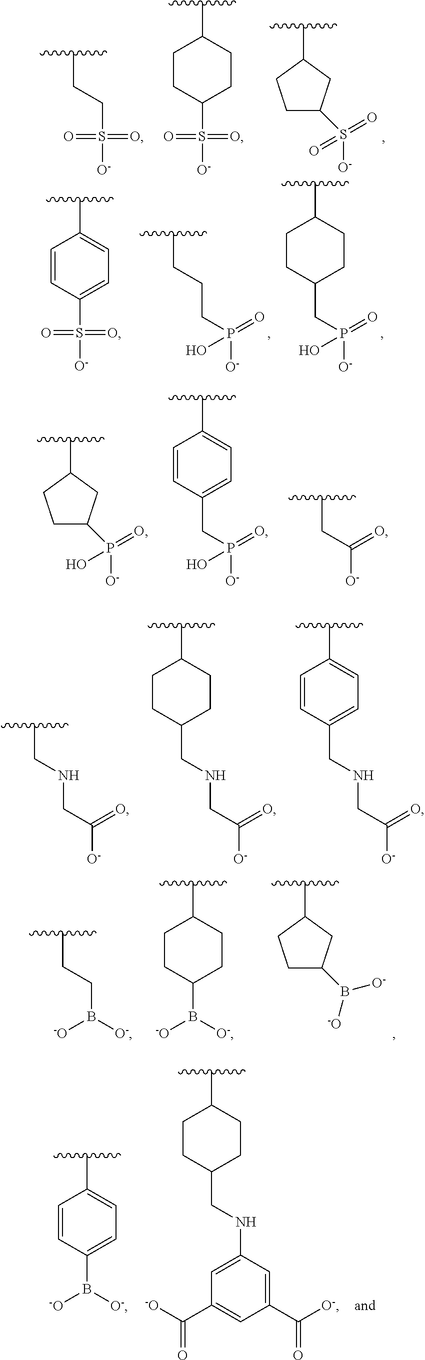

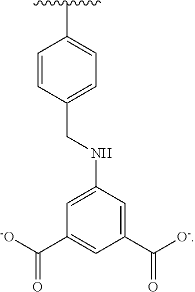

12. The catalyst of any one of embodiments 1 to 11, wherein the polymeric backbone is selected from the group consisting of polyethylene, polypropylene, polyvinyl alcohol, polystyrene, polyurethane, polyvinyl chloride, polyphenol-aldehyde, polytetrafluoroethylene, polybutylene terephthalate, polycaprolactam, poly(acrylonitrile butadiene styrene), polyalkyleneammonium, polyalkylenediammonium, polyalkylenepyrrolium, polyalkyleneimidazolium, polyalkylenepyrazolium, polyalkyleneoxazolium, polyalkylenethiazolium, polyalkylenepyridinium, polyalkylenepyrimidinium, polyalkylenepyrazinium, polyalkylenepyradizimium, polyalkylenethiazinium, polyalkylenemorpholinium, polyalkylenepiperidinium, polyalkylenepiperizinium, polyalkylenepyrollizinium, polyalkylenetriphenylphosphonium, polyalkylenetrimethylphosphonium, polyalkylenetriethylphosphonium, polyalkylenetripropylphosphonium, polyalkylenetributylphosphonium, polyalkylenetrichlorophosphonium, polyalkylenetrifluorophosphonium, and polyalkylenediazolium.
13. The catalyst of any one of embodiments 1 to 12, wherein the catalyst is cross-linked.
14. The catalyst of any one of embodiments 1 to 13, wherein the basic monomers and the ionic monomers are randomly arranged in an alternating sequence or in blocks of monomers.
15. The catalyst of embodiment 14, wherein each block has no more than twenty monomers.
16. The catalyst of any one of embodiments 1 to 15, further comprising hydrophobic monomers connected to form the polymeric backbone.
17. The catalyst of embodiment 16, wherein each hydrophobic monomer is selected from the group consisting of an unsubstituted or substituted alkyl, an unsubstituted or substituted cycloalkyl, an unsubstituted or substituted aryl, and an unsubstituted or substituted heteroaryl.
18. The catalyst of any one of embodiments 1 to 17, further comprising basic-ionic monomers connected to form the polymeric backbone, wherein each basic-ionic monomer comprises at least one Bronsted-Lowry base and at least one anionic group.
19. The catalyst of embodiment 18, wherein each Bronsted-Lowry base independently comprises at least one nitrogen-containing cationic group, at least one phosphorous-containing cationic group, at least one sulfur-containing cationic group, or any combinations thereof.
20. The catalyst of embodiment 18, wherein each Bronsted-Lowry base is selected from the group consisting of pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.
21. The catalyst of any one of embodiments 18 to 20, wherein each anionic group is independently selected from the group consisting of sulfonate, phosphonate, acetate, isophthalate, and boronate.
22. The catalyst of any one of embodiments 18 to 21, wherein one or more of the anionic groups are directly connected to polymeric backbone.
23. The catalyst of any one of embodiments 18 to 22, wherein one or more of the anionic groups are connected to the polymeric backbone by a linker.
24. The catalyst of embodiment 23, wherein each linker is independently selected from unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate.
25. The catalyst of embodiment 23, wherein at least one Bronsted-Lowry base, at least one anionic group, and a linker form a side chain, and wherein each side chain comprises:

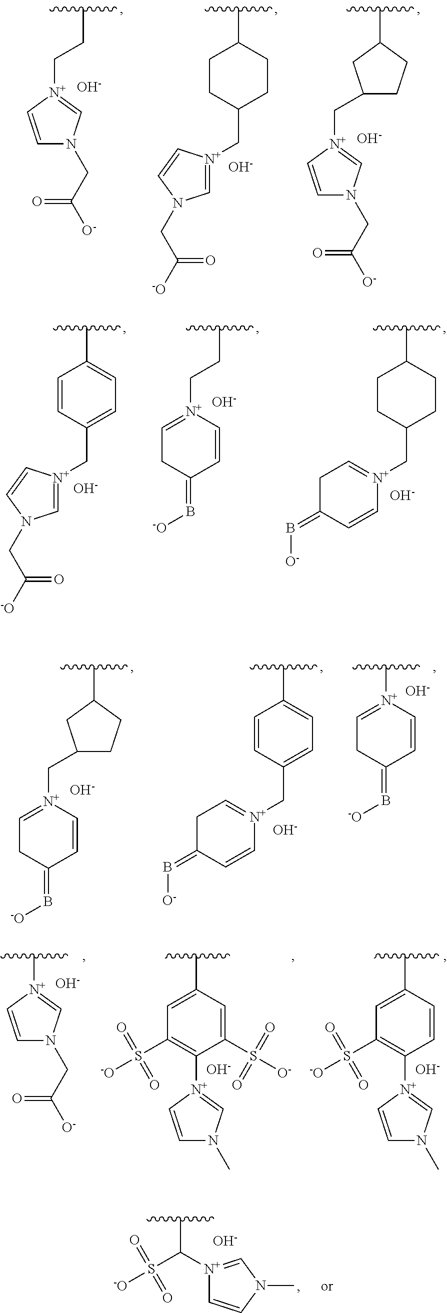

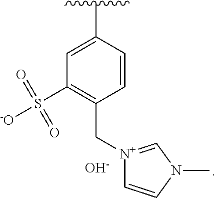

26. A catalyst comprising a solid support, basic moieties attached to the solid support, and ionic moieties attached to the solid support,

wherein the solid support comprises a material, wherein the material is selected from the group consisting of carbon, silica, silica gel, alumina, magnesia, titania, zirconia, clays, magnesium silicate, silicon carbide, zeolites, ceramics, and any combinations thereof,

wherein each basic moiety independently comprises at least one Bronsted-Lowry base, wherein each Bronsted-Lowry base independently comprises at least one nitrogen-containing cationic group, at least one phosphorous-containing cationic group, at least one sulfur-containing cationic group, or any combinations thereof.

27. The catalyst of embodiment 26, wherein the material is carbon.
28. The catalyst of embodiment 27, wherein the carbon is selected from the group consisting of biochar, amorphous carbon, and activated carbon.
29. The catalyst of any one of embodiments 26 to 28, wherein each Bronsted-Lowry base is selected from the group consisting of pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.
30. The catalyst of any one of embodiments 26 to 29, wherein one or more of the basic moieties are directed attached to the solid support.
31. The catalyst of any one of embodiments 26 to 30, wherein one or more of the basic moieties are attached to the solid support by a linker.
32. The catalyst of embodiment 31, wherein each linker is independently selected from the group consisting of unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate.
33. The catalyst of embodiment 26, wherein each basic moiety is independently selected from the group consisting of:

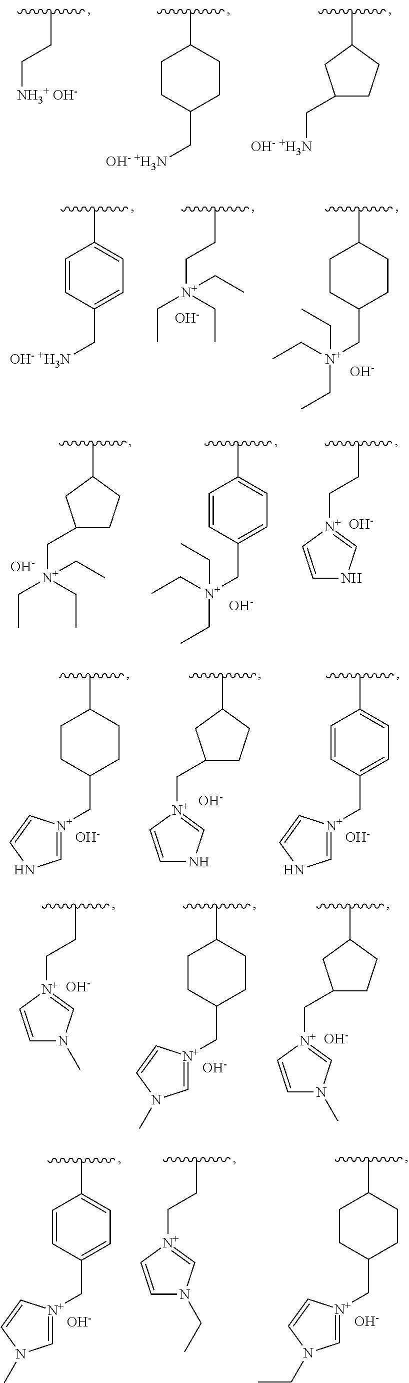

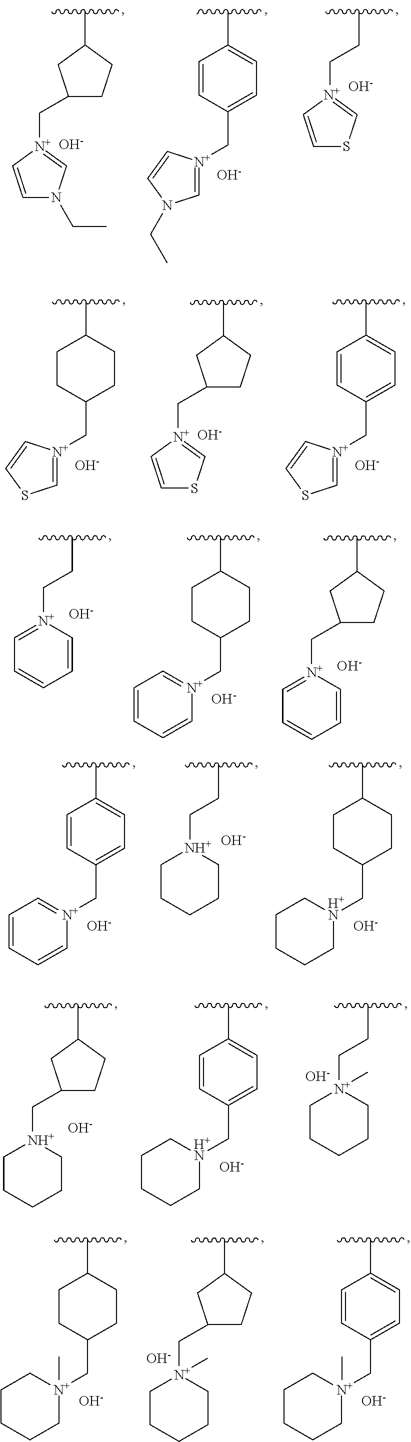

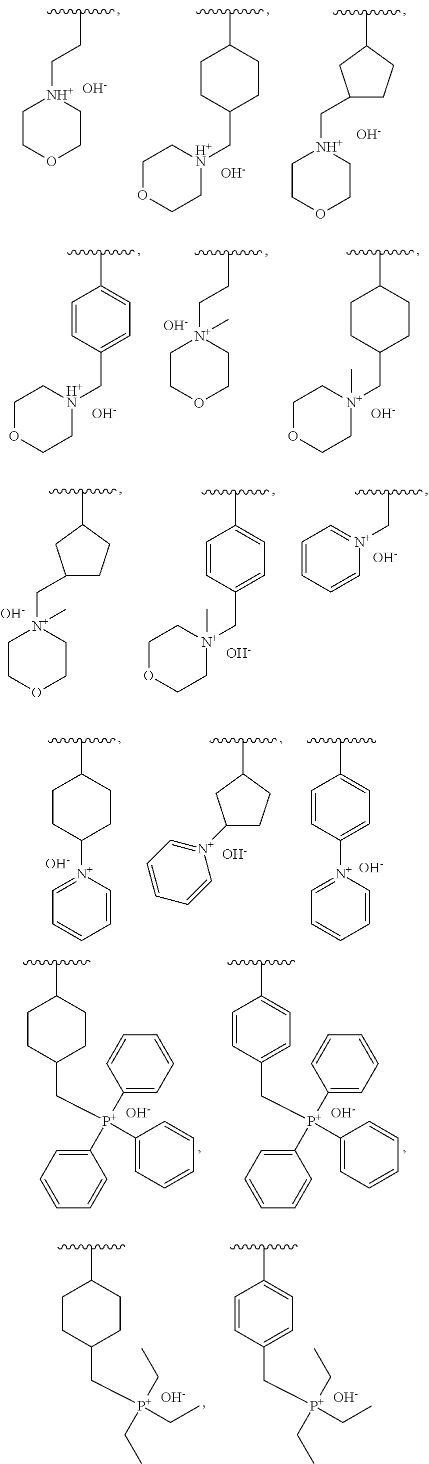

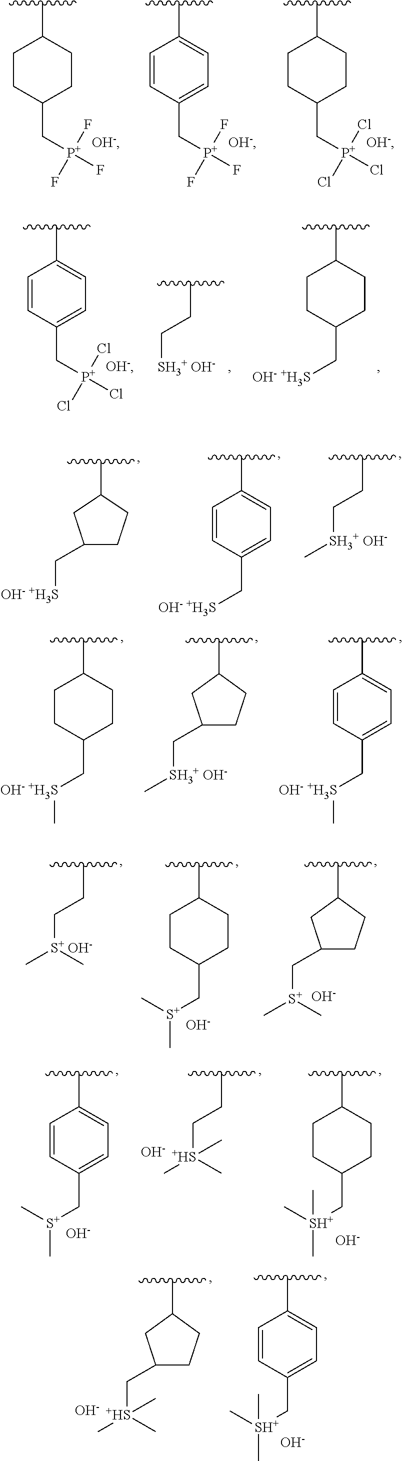

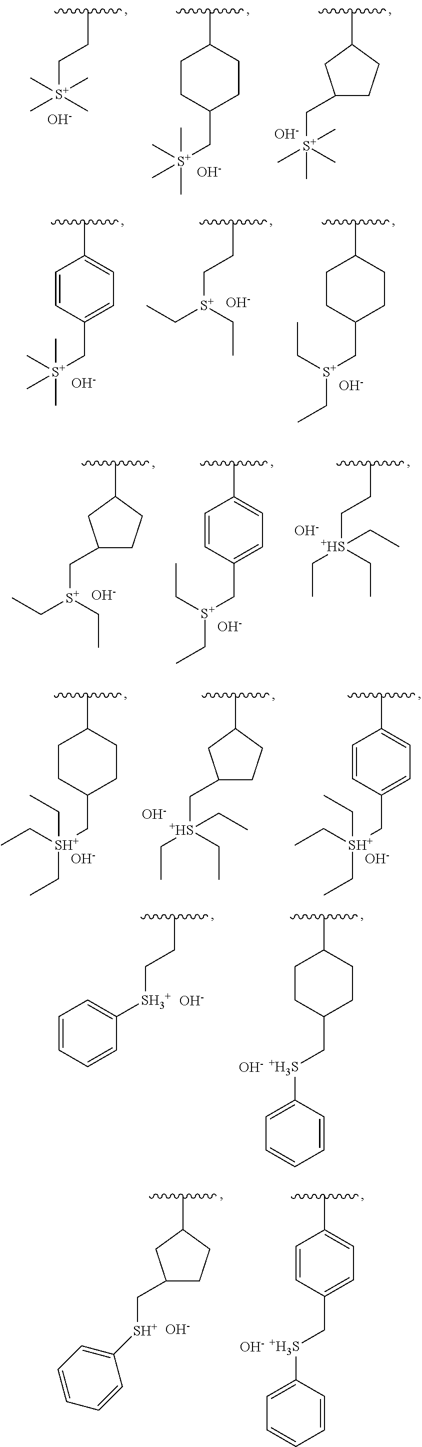

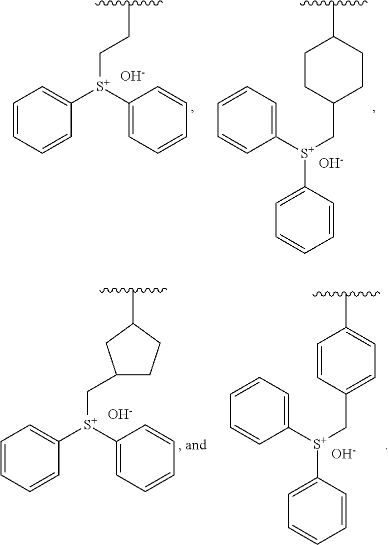

34. The catalyst of any one of embodiments 26 to 33, wherein each ionic moiety is independently selected from the group consisting of a sulfonate salt, a phosphonate salt, an acetate salt, an isophthalate salt, and a boronate salt.
35. The catalyst of any one of embodiments 26 to 34, wherein one or more of the ionic moieties are directly attached to the solid support.
36. The catalyst of any one of embodiments 26 to 35, wherein one or more of the ionic moieties are attached to the solid support by a linker.
37. The catalyst of embodiment 36, wherein each linker is independently selected from the group consisting of unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate.
38. The catalyst of embodiment 26, wherein each ionic moiety comprises at least one anionic group selected from the group consisting of:

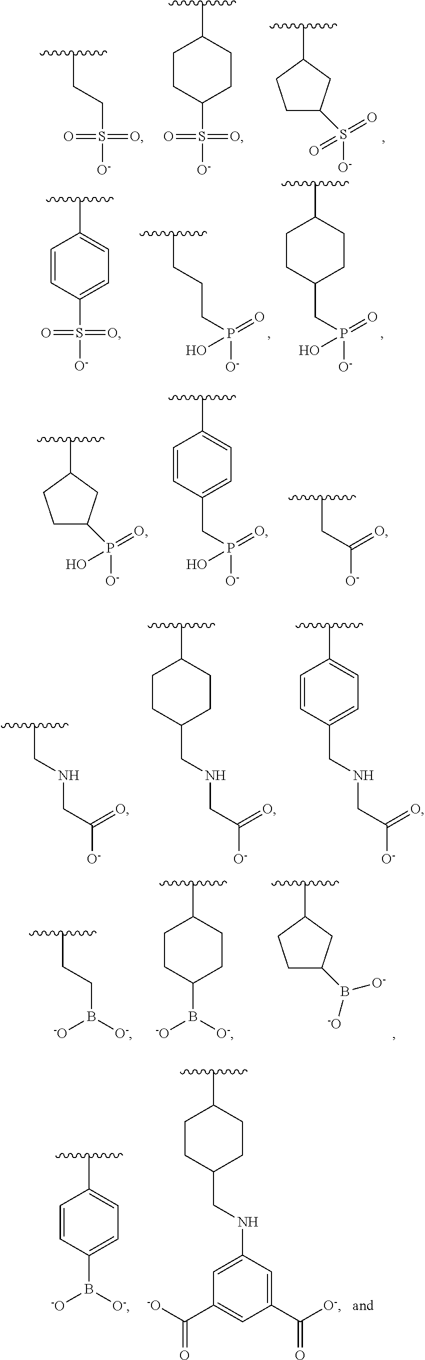

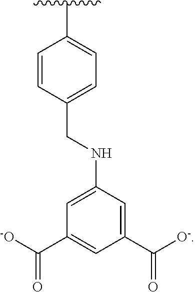

39. The catalyst of any one of embodiments 26 to 38, further comprising hydrophobic moieties attached to the solid support.
40. The catalyst of embodiment 39, wherein each hydrophobic moiety is selected from the group consisting of an unsubstituted or substituted alkyl, an unsubstituted or substituted cycloalkyl, an unsubstituted or substituted aryl, and an unsubstituted or substituted heteroaryl.
41. The catalyst of any one of embodiments 26 to 40, further comprising basic-ionic moieties attached to the solid support, wherein each basic-ionic moiety comprises at least one Bronsted-Lowry base and at least one anionic group.
42. The catalyst of embodiment 41, wherein each Bronsted-Lowry base independently comprises at least one nitrogen-containing cationic group, at least one phosphorous-containing cationic group, at least one sulfur-containing cationic group, or any combinations thereof.
43. The catalyst of embodiment 41, wherein each Bronsted-Lowry base is selected from the group consisting of pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.
44. The catalyst of any one of embodiments 41 to 43, wherein each anionic group is independently selected from the group consisting of sulfonate, phosphonate, acetate, isophthalate, and boronate.
45. The catalyst of any one of embodiments 41 to 44, wherein one or more of the basic-ionic moieties are directly attached to the solid support.
46. The catalyst of any one of embodiments 41 to 44, wherein one or more of the basic-ionic moieties are attached to the solid support by a linker.
47. The catalyst of embodiment 46, wherein each linker is independently selected from unsubstituted or substituted alkyl linker, unsubstituted or substituted cycloalkyl linker, unsubstituted or substituted alkenyl linker, unsubstituted or substituted aryl linker, unsubstituted or substituted heteroaryl linker, unsubstituted or substituted alkyl linker ether, unsubstituted or substituted alkyl linker ester, and unsubstituted or substituted alkyl linker carbamate.
48. The catalyst of embodiment 41, wherein each basic-ionic moiety independently comprises:

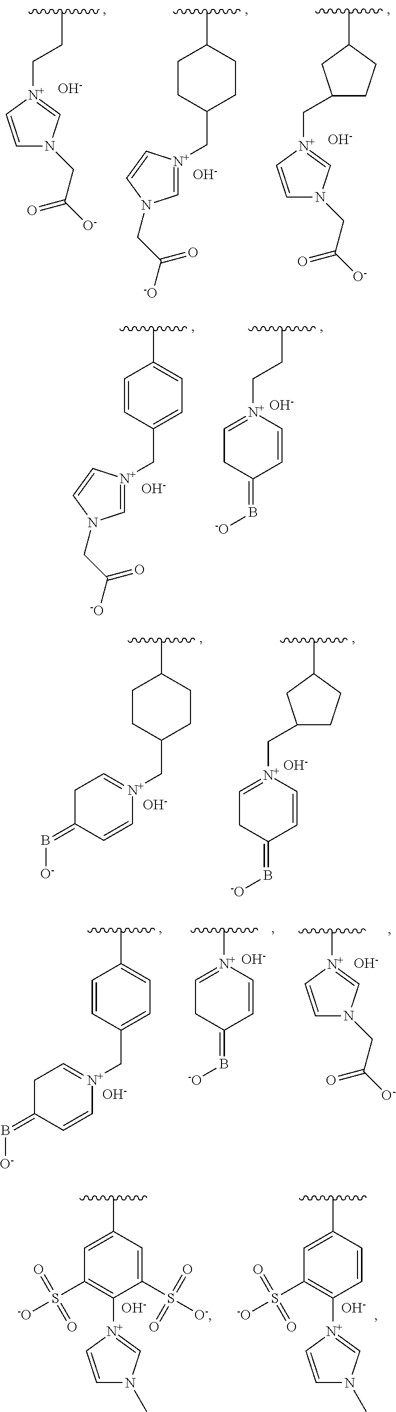

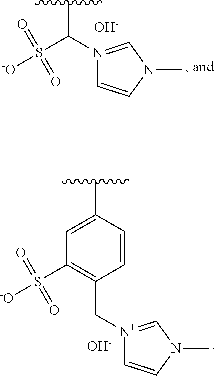

49. The catalyst of any one of embodiments 26 to 48, wherein the catalyst has a total amount of Bronsted-Lowry base of between 0.01 mmol and 5.0 mmol per gram of the catalyst.
50. The catalyst of any one of embodiments 26 to 49, wherein the catalyst has a total amount of anionic groups of between 0.01 mmol and 5.0 mmol per gram of the catalyst.
51. A composition comprising:

lignin; and

a catalyst of any one of embodiments 1 to 50.

52. A partially-depolymerized lignin composition comprising:

a catalyst of any one of embodiments 1 to 50;

one or more lignin digestion products; and

residual lignin.

53. The composition of embodiment 52, wherein the one or more lignin digestion products are selected from the group consisting of monolignols, phenylpropenes, monolignolglucosides, and any combinations thereof.
54. The composition of embodiment 52, wherein the one or more lignin digestion products are selected from the group consisting of p-coumaryl alcohol, coumarilin, coniferyl alchol, coniferin, sinapyl alcohol, sinaplin, eugenol, chavicol, safrole, estragol, and any combinations thereof.
55. A method for at least partially depolymerizing a lignin composition, comprising:

a) providing a lignin composition;

b) contacting the lignin composition with a catalyst of any one of embodiments 1 to 50 and one or more solvents to form a reaction mixture;

c) degrading the lignin composition in the reaction mixture to produce a liquid phase and a solid phase, wherein the liquid phase comprises one or more lignin digestion products, and the solid phase comprises residual lignin;

d) isolating at least a portion of the liquid phase from the solid phase; and

e) recovering the one or more lignin digestion products from the isolated liquid phase.

56. The method of embodiment 55, wherein the one or more lignin digestion products are selected from the group consisting of monolignols, phenylpropenes, monolignolglucosides, and any combinations thereof.
57. The method of embodiment 55, wherein the one or more lignin digestion products are selected from the group consisting of p-coumaryl alcohol, coumarilin, coniferyl alchol, coniferin, sinapyl alcohol, sinaplin, eugenol, chavicol, safrole, estragol, and any combinations thereof.
58. Use of a catalyst of any one of embodiments 1 to 50 for degrading a lignin composition into one or more lignin digestion products.
59. A method of producing a catalyst of any one of embodiments 1 to 25, comprising:

a) providing a starting polymer;

b) reacting the starting polymer with a nitrogen-containing compound, a phosphorous-containing compound, or a sulfur-containing compound to produce an ionic intermediate;

c) reacting the ionic intermediate with an acid to produce an acidic/ionic intermediate; and

d) washing the acidic/ionic intermediate with a base to produce the catalyst of any one of embodiments 1 to 25.

60. The method of embodiment 59, wherein the starting polymer is selected from the group consisting of polyethylene, polypropylene, polyvinyl alcohol, polycarbonate, polystyrene, polyurethane, or a combination thereof.
61. The method of embodiment 59, wherein the starting polymer is a polystyrene.
62. The method of embodiment 61, wherein the starting polymer is poly(styrene-co-vinylbenzylhalide-co-divinylbenzene).
63. The method of embodiment 62, wherein the starting polymer is poly(styrene-co-vinylbenzylchloride-co-divinylbenzene).
64. The method of any one of embodiments 59 to 63, wherein the nitrogen-containing compound is selected from the group consisting of a pyrrolium compound, an imidazolium compound, a pyrazolium compound, an oxazolium compound, a thiazolium compound, a pyridinium compound, a pyrimidinium compound, a pyrazinium compound, a pyradizimium compound, a thiazinium compound, a morpholinium compound, a piperidinium compound, a piperizinium compound, and a pyrollizinium compound.
65. The method of any one of embodiments 59 to 64, wherein the acid is selected from the group consisting of sulfuric acid, phosphonic acid, hydrochloric acid, acetic acid and boronic acid.
66. The method of any one of embodiments 59 to 65, wherein the base is selected from the group consisting of sodium hydroxide, potassium hydroxide, magnesium hydroxide, calcium hydroxide, lead hydroxide, and ammonium hydroxide.
67. A catalyst produced according to the method of any one of embodiments 59 to 66.
68. A method for producing a catalyst of any one of embodiments 26 to 50, comprising:

a) providing a carbonaceous material;

b) carbonizing at least a portion of the carbonaceous material to form a solid support;

b) activating at least a portion of the solid support;

c) functionalizing the activated solid support with one or more cationic groups to form a quaternized solid support, wherein each cationic group is independently a nitrogen-containing cationic group, a phosphorous-containing cationic group, a sulfur-containing cationic group, or any combination thereof;

d) functionalizing the quaternized solid support with one or more acidic groups to form a quaternized-acidic solid support, wherein each acidic group is independently a Bronsted-Lowry acid; and

e) washing the quaternized/acidic solid support with a base to produce the catalyst of any one of embodiments 26 to 50.

69. A method for producing a catalyst of any one of embodiments 26 to 50, comprising:

a) providing a carbonaceous material;

b) carbonizing at least a portion of the carbonaceous material to form a solid support;

b) activating at least a portion of the solid support;

c) functionalizing the activated solid support with one or more acidic groups to form an acidified solid support, wherein each acidic group is independently a Bronsted-Lowry acid;

d) functionalizing the acidified solid support with one or more cationic groups to form a quaternized-acidic solid support, wherein each cationic group is independently a nitrogen-containing cationic group, a phosphorous-containing cationic group, or a sulfur-containing cationic group; and

e) washing the quaternized-acidic solid support with a base to produce the catalyst of any one of embodiments 26 to 50.

70. The method of embodiment 68 or 69, wherein the carbonaceous material is selected from the group consisting of shrimp shell, chitin, coconut shell, wood pulp, paper pulp, cotton, cellulose, hard wood, soft wood, wheat straw, sugarcane bagasse, cassava stem, corn stover, oil palm residue, bitumen, asphaltum, tar, coal, pitch, and any combinations thereof.
71. The method of any one of embodiments 68 to 70, wherein the carbonaceous material has a carbon content of greater than 20% g carbon/g dry carbonaceous material.
72. The method of any one of embodiments 68 to 71, wherein the carbonaceous material is carbonized by pyrolysis.
73. The method of any one of embodiments 68 to 72, wherein the carbonaceous material is carbonized in an atmosphere containing less than 20% of oxygen.
74. The method of any one of embodiments 68 to 73, wherein the carbonaceous material is carbonized at a temperature between 200° C. and 500° C.
75. The method of any one of embodiments 68 to 74, wherein the activating of at least a portion of the solid support comprises:

contacting the solid support with a choromethylating agent or chloroacylating agent to attach a reactive linker to the solid support.

76. The method of embodiment 75, wherein the reactive linker is selected from the group consisting of a haloalkane, a haloacyl, an amine or a diazo.
77. The method of embodiment 75 or 76, wherein the chloromethylating agent is chloromethyl methyl ether.
78. The method of embodiment 75 or 76, wherein the chloroacylating agent is chloroacetyl chloride.
79. The method of any one of embodiments 68 to 78, wherein the base is selected from the group consisting of sodium hydroxide, potassium hydroxide, magnesium hydroxide, calcium hydroxide, lead hydroxide, and ammonium hydroxide.

### EXAMPLES

Except where otherwise indicated, commercial reagents were obtained from Sigma-Aldrich, St. Louis, Mo., USA, and were purified prior to use following the guidelines of Perrin and Armarego. See Perrin, D. D. & Armarego, W. L. F., Purification of Laboratory Chemicals, 3rd ed.; Pergamon Press, Oxford, 1988. Nitrogen gas for use in chemical reactions was of ultra-pure grade, and was dried by passing it through a drying tube containing phosphorous pentoxide. Unless indicated otherwise, all non-aqueous reagents were transferred under an inert atmosphere via syringe or Schlenk flask. Organic solutions were concentrated under reduced pressure on a Buchi rotary evaporator. Where necessary, chromatographic purification of reactants or products was accomplished using forced-flow chromatography on 60 mesh silica gel according to the method described of Still et al., See Still et al., J. Org. Chem., 43: 2923 (1978). Thin-layer chromatography (TLC) was performed using silica-coated glass plates. Visualization of the developed chromatogram was performed using either Cerium Molybdate (i.e., Hanessian) stain or KMnO₄ stain, with gentle heating, as required. Fourier-Transform Infrared (FTIR) spectroscopic analysis of solid samples was performed on a Perkin-Elmer 1600 instrument equipped with a horizontal attenuated total reflectance (ATR) attachment using a Zinc Selenide (ZnSe) crystal.

### Preparation of Polymeric Catalysts

### Example A1

### Preparation of poly[styrene-co-vinylbenzylchloride-co-divinylbenzene]

To a 500 mL round bottom flask (RBF) containing a stirred solution of 1.08 g of poly(vinylalcohol) in 250.0 mL of deionized H₂O at 0° C., was gradually added a solution containing 50.04 g (327.9 mmol) of vinylbenzyl chloride (mixture of 3- and 4-isomers), 10.13 g (97.3 mmol) of styrene, 1.08 g (8.306 mmol) of divinylbenzene (DVB, mixture of 3- and 4-isomers) and 1.507 g (9.2 mmol) of azobisisobutyronitrile (AIBN) in 150 mL of a 1:1 (by volume) mixture of benzene/tetrahydrofuran (THF) at 0° C. After 2 hours of stirring at 0° C. to homogenize the mixture, the reaction flask was transferred to an oil bath to increase the reaction temperature to 75° C., and the mixture was stirred vigorously for 28 hours. The resulting polymer beads were vacuum filtered using a fritted-glass funnel to collect the polymer product. The beads were washed repeatedly with 20% (by volume) methanol in water, THF, and MeOH, and dried overnight at 50° C. under reduced pressure to yield 59.84 g of polymer. The polymer beads were separated by size using sieves with mesh sizes 100, 200, and 400.

### Example A2

### Preparation of poly[styrene-co-3-methyl-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 50 g, 200 mmol) was charged into a 500 mL three neck flask (TNF) equipped with a mechanical stirrer, a dry nitrogen line, and purge valve. Dry dimethylformamide (185 ml) was added into the flask (via cannula under N₂) and stirred to form a viscous slurry of polymer resin. 1-Methylimidazole (36.5 g, 445 mmol) was then added and stirred at 95° C. for 8 h. After cooling, the reaction mixture was filtered using a fritted glass funnel under vacuum, washed sequentially with de-ionized water and ethanol, and finally air dried.

The chemical functionalization of the polymer material, expressed in millimoles of functional groups per gram of dry polymer resin (mmol/g) was determined by ion exchange titrimetry. For the determination of cation-exchangable acidic protons, a known dry mass of polymer resin was added to a saturated aqueous solution of sodium chloride and titrated against a standard sodium hydroxide solution to the phenolphthalein end point. For the determination of anion-exchangeable ionic chloride content, a known dry mass of polymer resin was added to an aqueous solution of sodium nitrate and neutralized with sodium carbonate. The resulting mixture was titrated against a standardized solution of silver nitrate to the potassium chromate endpoint. For polymeric materials in which the exchangeable anion was not chloride, the polymer was first treated by stirring the material in aqueous hydrochloric acid, followed by washing repeatedly with water until the effluent was neutral (as determined by pH paper). The chemical functionalization of the polymer resin with methylimidazolium chloride groups was determined to be 2.60 mmol/g via gravimetry and 2.61 mmol/g via titrimetry.

### Example A3

### Preparation of poly[styrene-co-4-vinylbenzenesulfonic acid-co-3-methyl-1-(4-vinylbenzyl)-3H-imidazol-1-ium bisulfate-co-divinylbenzene]

Poly[styrene-co-3-methyl-1-(4-vinylbenzyl)-3H-imidazol-1-iumchloride-co-divinylbenzene] (63 g) was charged into a 500 mL flask equipped with a magnetic stir bar and condenser. Cold concentrated sulfuric acid (>98% w/w, H₂SO₄, 300 mL) was gradually added into the flask under stirring which resulted in formation of dark-red colored slurry of resin. The slurry was stirred at 85° C. for 4 h. After cooling to room temperature, the reaction mixture was filtered using fritted glass funnel under vacuum and then washed repeatedly with de-ionized water until the effluent was neutral, as determined by pH paper. The sulfonated resin beads were finally washed with ethanol and air dried. The chemical functionalization of the polymer resin with sulfonic acid groups was determined to be 1.60 mmol/g, as determined by titrimetry following the procedure of Example A2.

### Example A4

### Preparation of poly[styrene-co-4-vinylbenzenesulfonic acid-co-3-methyl-1-(4-vinylbenzyl)-3H-imidazol-1-ium chloride-co-divinylbenzene]

Poly[styrene-co-4-vinylbenzenesulfonic acid-co-3-methyl-1-(4-vinylbenzyl)-3H-imidazol-1-ium bisulfate-co-divinylbenzene] (sample of Example A3), contained in fritted glass funnel, was washed repeatedly with 0.1 M HCl solution to ensure complete exchange of HSO₄⁻ with AcO⁻. The resin was then washed with de-ionized water until the effluent was neutral, as determined by pH paper. The resin was finally air-dried.

### Example A5

### Preparation of poly[styrene-co-4-vinylbenzenesulfonic acid-co-3-methyl-1-(4-vinylbenzyl)-3H-imidazol-1-ium acetate-co-divinylbenzene]

The suspension of poly[styrene-co-4-vinylbenzenesulfonic acid-co-3-methyl-1-(4-vinylbenzyl)-3H-imidazol-1-ium bisulfate-co-divinylbenzene] (sample of Example A3) in 10% aqueous acetic acid solution was stirred for 2 h at 60° C. to ensure complete exchange of HSO₄⁻ with AcO⁻. The resin was filtered using fritted glass funnel and then washed multiple times with de-ionized water until the effluent was neutral. The resin was finally air-dried.

### Example A6

### Preparation of poly[styrene-co-3-ethyl-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 10 g, 40 mmol) was charged into a 250 three neck flask (TNF) equipped with a mechanical stirrer, a dry nitrogen line, and purge valve. Dry dimethylformamide (80 ml) was added into the flask (via cannula under N₂) and stirred to give viscous resin slurry. 1-Ethylimidazole (4.3 g, 44.8 mmol) was then added to the resin slurry and stirred at 95° C. under 8 h. After cooling, the reaction mixture was filtered using fritted glass funnel under vacuum, washed sequentially with de-ionized water and ethanol, and finally air dried. The chemical functionalization of the polymer resin with ethylimidazolium chloride groups was determined to be 1.80 mmol/g, as determined by titrimetry following the procedure of Example A1.

### Example A7

### Preparation of poly[styrene-co-4-vinylbenzenesulfonic acid-co-3-ethyl-1-(4-vinylbenzyl)-3H-imidazol-1-ium bisulfate-co-divinylbenzene]

Poly[styrene-co-3-ethyl-1-(4-vinylbenzyl)-3H-imidazol-1-ium chloride-co-divinylbenzene] (5 g) was charged into a 100 mL flask equipped with a magnetic stir bar and condenser. Cold concentrated sulfuric acid (>98% w/w, H₂SO₄, 45 mL) was gradually added into the flask under stirring which resulted in the formation of dark-red colored uniform slurry of resin. The slurry was stirred at 95-100° C. for 6 h. After cooling, the reaction mixture was filtered using fritted glass funnel under vacuum and then washed repeatedly with de-ionized water until the effluent was neutral, as determined by pH paper. The sulfonated beads were finally washed with ethanol and air dried. The chemical functionalization of the polymer with sulfonic acid groups was determined to be 1.97 mmol/g, as determined by titrimetry following the procedure of Example A2.

### Example A8

### Preparation of poly[styrene-co-4-vinylbenzenesulfonic acid-co-3-ethyl-1-(4-vinylbenzyl)-3H-imidazol-1-ium chloride-co-divinylbenzene]

Poly[styrene-co-4-vinylbenzenesulfonic acid-co-3-ethyl-1-(4-vinylbenzyl)-3H-imidazol-1-ium bisulfate-co-divinylbenzene] resin beads (sample of Example A7) contained in fritted glass funnel was washed multiple times with 0.1 M HCl solution to ensure complete exchange of HSO₄⁻ with Cl⁻. The resin was then washed with de-ionized water until the effluent was neutral, as determined by pH paper. The resin was finally washed with ethanol and air dried.

### Example A9

### Preparation of poly[styrene-co-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Chloroform (50 ml) was added into the flask and stirred to form slurry of resin. Imidazole (2.8 g, 41.13 mmol) was then added to the resin slurry and stirred at 40° C. for 18 h. After completion of reaction, the reaction mixture was filtered using fritted glass funnel under vacuum, washed sequentially with de-ionized water and ethanol, and finally air dried. The chemical functionalization of the polymer resin with imidazolium chloride groups was determined to be 2.7 mmol/g, as determined by titrimetry following the procedure of Example A2.

### Example A10

### Preparation of poly[styrene-co-4-vinylbenzenesulfonic acid-co-1-(4-vinylbenzyl)-3H-imidazol-1-ium bisulfate-co-divinylbenzene]

Poly[styrene-co-1-(4-vinylbenzyl)-3H-imidazol-1-ium chloride-co-divinylbenzene] (5 g) was charged into a 100 mL flask equipped with a magnetic stir bar and condenser. Cold concentrated sulfuric acid (>98% w/w, H₂SO₄, 80 mL) was gradually added into the flask and stirred to form dark-red colored slurry of resin. The slurry was stirred at 95° C. for 8 h. After cooling, the reaction mixture was filtered using fritted glass funnel under vacuum and then washed repeatedly with de-ionized water until the effluent was neutral, as determined by pH paper. The sulfonated beads were finally washed with ethanol and air dried. The chemical functionalization of the polymer resin with sulfonic acid groups was determined to be 1.26 mmol/g, as determined by titrimetry following the procedure of Example A2.

### Example A11

### Preparation of poly[styrene-co-3-methyl-1-(4-vinylbenzyl)-3H-benzoimidazol-1-ium chloride-co-divinylbenzene]

Poly(styrene-co-vinylbenzylchloride-co-divinylbenzene) (Cl⁻ density=˜4.0 mmol/g, 4 g, 16 mmol) was charged into a 100 mL flask equipped with a magnetic stir bar and condenser. Dry dimethylformamide (50 ml) was added into the flask (via cannula under N₂) and stirred to form viscous slurry of polymer resin. 1-Methylbenzimidazole (3.2 g, 24.2 mmol) was then added to the resin slurry and the resulting reaction mixture was stirred at 95° C. for 18 h. After cooling, the reaction mixture was filtered using fritted glass funnel under vacuum, washed sequentially with de-ionized water and ethanol, and finally air dried. The chemical functionalization of the polymer with methylbenzimidazolium chloride groups was determined to be 1.63 mmol/g, as determined by titrimetry following the procedure of Example A2.

### Example A12

### Preparation of poly[styrene-co-4-vinylbenzenesulfonic acid-co-3-methyl-1-(4-vinylbenzyl)-3H-benzoimidazol-1-ium bisulfate-co-divinylbenzene]

Poly[styrene-co-3-methyl-1-(4-vinylbenzyl)-3H-benzoimidazol-1-ium chloride-co-divinylbenzene] (5.5 g) was charged into a 100 mL flask equipped with a magnetic stir bar and condenser. Cold concentrated sulfuric acid (>98% w/w, H₂SO₄, 42 mL) and fuming sulfuric acid (20% free SO₃, 8 mL) was gradually added into the flask and stirred to form dark-red colored slurry of resin. The slurry was stirred at 85° C. for 4 h. After cooling, the reaction mixture was filtered using fritted glass funnel under vacuum and then washed repeatedly with de-ionized water until the effluent was neutral, as determined by pH paper. The sulfonated beads were finally washed with ethanol and air dried. The chemical functionalization of the polymer with sulfonic acid groups was determined to be 1.53 mmol/g, as determined by titrimetry following the procedure of Example A2.

### Example A13

### Preparation of poly[styrene-co-1-(4-vinylbenzyl)-pyridinium chloride-co-divinylbenzene]

Poly(styrene-co-vinylbenzylchloride-co-divinylbenzene) (Cl⁻ density=˜4.0 mmol/g, 5 g, 20 mmol) was charged into a 100 mL flask equipped with a magnetic stir bar and condenser. Dry dimethylformamide (45 ml) was added into the flask (via cannula under N₂) while stirring and consequently, the uniform viscous slurry of polymer resin was obtained. Pyridine (3 mL, 37.17 mmol) was then added to the resin slurry and stirred at 85-90° C. for 18 h. After cooling, the reaction mixture was filtered using fritted glass funnel under vacuum, washed sequentially with de-ionized water and ethanol, and finally air dried. The chemical functionalization of the polymer resin with pyridinium chloride groups was determined to be 3.79 mmol/g, as determined by titrimetry following the procedure of Example A2.

### Example A14

### Preparation of poly[styrene-co-4-vinylbenzenesulfonic acid-co-1-(4-vinylbenzyl)-pyridinium-bisulfate-co-divinylbenzene]

Poly[styrene-co-1-(4-vinylbenzyl)-pyridinium chloride-co-divinylbenzene] (4 g) resin beads were charged into a 100 mL flask equipped with a magnetic stir bar and condenser. Cold concentrated sulfuric acid (>98% w/w, H₂SO₄, 45 mL) was gradually added into the flask under stirring which consequently resulted in the formation of dark-red colored uniform slurry of resin. The slurry was heated at 95-100° C. under continuous stirring for 5 h. After completion of reaction, the cooled reaction mixture was filtered using fritted glass funnel under vacuum and then washed repeatedly with de-ionized water until the effluent was neutral, as determined by pH paper. The resin beads were finally washed with ethanol and air dried. The chemical functionalization of the polymer with sulfonic acid groups was determined to be 0.64 mmol/g, as determined by titrimetry following the procedure of Example A2.

### Example A15

### Preparation of poly[styrene-co-1-(4-vinylbenzyl)-pyridinium chloride-co-3-methyl-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (80 ml) was added into the flask (via cannula under N₂) while stirring which resulted in the formation of viscous slurry of polymer resin. Pyridine (1.6 mL, 19.82 mmol) and 1-methylimidazole (1.7 mL, 21.62 mmol) were then added to the resin slurry and the resulting reaction mixture was stirred at 95° C. for 18 h. After completion of reaction, the reaction mixture was cooled, filtered using fritted glass funnel under vacuum, washed sequentially with de-ionized water and ethanol, and finally air dried. The chemical functionalization of the polymer with pyridinium chloride and 1-methylimidazolium chloride groups was determined to be 3.79 mmol/g, as determined by titrimetry following the procedure of Example A2.

### Example A16

### Preparation of poly[styrene-co-4-vinylbenzenesulfonic acid-co-1-(4-vinylbenzyl)-pyridiniumchloride-co-3-methyl-1-(4-vinylbenzyl)-3H-imidazol-1-ium bisulfate-co-divinylbenzene]

Poly[styrene-co-1-(4-vinylbenzyl)-pyridinium chloride-co-3-methyl-1-(4-vinylbenzyl)-3H-imidazol-1-ium chloride-co-divinylbenzene] (5 g) was charged into a 100 mL flask equipped with a magnetic stir bar and condenser. Cold concentrated sulfuric acid (>98% w/w, H₂SO₄, 75 mL) and fuming sulfuric acid (20% free SO₃, 2 mL) were then gradually added into the flask under stirring which consequently resulted in the formation of dark-red colored uniform slurry of resin. The slurry was heated at 95-100° C. under continuous stirring for 12 h. After completion of reaction, the cooled reaction mixture was filtered using fritted glass funnel under vacuum and then washed repeatedly with de-ionized water until the effluent was neutral, as determined by pH paper. The sulfonated resin beads were finally washed with ethanol and air dried. The chemical functionalization of the polymer resin with sulfonic acid groups was determined to be 1.16 mmol/g, as determined by titrimetry following the procedure of Example A2.

### Example A17

### Preparation of poly[styrene-co-4-methyl-4-(4-vinylbenzyl)-morpholin-4-ium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (85 ml) was added into the flask (via cannula under N₂) while stirring which resulted in the formation of uniform viscous slurry of polymer resin. 1-Methylmorpholine (5.4 mL, 49.12 mmol) were then added to the resin slurry and the resulting reaction mixture was stirred at 95° C. for 18 h. After cooling, the reaction mixture was filtered using fritted glass funnel under vacuum, washed sequentially with de-ionized water and ethanol, and finally air dried. The chemical functionalization of the polymer with methylmorpholinium chloride groups was determined to be 3.33 mmol/g, as determined by titrimetry following the procedure of Example A2.

### Example A18

### Preparation of poly[styrene-co-4-vinylbenzenesulfonic acid-co-4-methyl-4-(4-vinylbenzyl)-morpholin-4-ium bisulfate-co-divinylbenzene]

Poly[styrene-co-1-4-methyl-4-(4-vinylbenzyl)-morpholin-4-ium chloride-co-divinylbenzene] (8 g) was charged into a 100 mL flask equipped with a magnetic stir bar and condenser. Cold concentrated sulfuric acid (>98% w/w, H₂SO₄, 50 mL) was gradually added into the flask under stirring which consequently resulted in the formation of dark-red colored slurry. The slurry was stirred at 90° C. for 8 h. After cooling, the reaction mixture was filtered using fritted glass funnel under vacuum, washed repeatedly with de-ionized water until the effluent was neutral, as determined by pH paper. The sulfonated resin beads were finally washed with ethanol and air dried. The chemical functionalization of the polymer with sulfonic acid groups was determined to be 1.18 mmol/g, as determined by titrimetry following the procedure of Example A2.

### Example A19

### Preparation of [polystyrene-co-triphenyl-(4-vinylbenzyl)-phosphoniumchloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (80 ml) was added into the flask (via cannula under N₂) while stirring and the uniform viscous slurry of polymer resin was obtained. Triphenylphosphine (11.6 g, 44.23 mmol) was then added to the resin slurry and the resulting reaction mixture was stirred at 95° C. for 18 h. After cooling, the reaction mixture was filtered using fritted glass funnel under vacuum, washed sequentially with de-ionized water and ethanol, and finally air dried. The chemical functionalization of the polymer with triphenylphosphonium chloride groups was determined to be 2.07 mmol/g, as determined by titrimetry following the procedure of Example A2.

### Example A20

### Preparation of poly[styrene-co-4-vinylbenzenesulfonic acid-co-triphenyl-(4-vinylbenzyl)-phosphonium bisulfate-co-divinylbenzene]

Poly (styrene-co-triphenyl-(4-vinylbenzyl)-phosphonium chloride-co-divinylbenzene) (7 g) was charged into a 100 mL flask equipped with a magnetic stir bar and condenser. Cold concentrated sulfuric acid (>98% w/w, H₂SO₄, 40 mL) and fuming sulfuric acid (20% free SO₃, 15 mL) were gradually added into the flask under stirring which consequently resulted in the formation of dark-red colored slurry. The slurry was stirred at 95° C. for 8 h. After cooling, the reaction mixture was filtered using fritted glass funnel under vacuum, washed repeatedly with de-ionized water until the effluent was neutral, as determined by pH paper. The sulfonated resin beads were finally washed with ethanol and air dried. The chemical functionalization of the polymer with sulfonic acid groups was determined to be 2.12 mmol/g, as determined by titrimetry following the procedure of Example A2.

### Example A21

### Preparation of poly[styrene-co-1-(4-vinylbenzyl)-piperidine-co-divinylbenzene]

Poly(styrene-co-vinylbenzyl chloride-co-divinylbenzene) (Cl⁻ density=˜4.0 mmol/g, 10 g, 40 mmol) was charged into a 100 mL flask equipped with a magnetic stir bar and condenser. Dry dimethylformamide (50 ml) was added into the flask (via cannula under N₂) while stirring which resulted in the formation of uniform viscous slurry of polymer resin. Piperidine (4 g, 46.98 mmol) was then added to the resin slurry and the resulting reaction mixture was stirred at 95° C. for 16 h. After cooling, the reaction mixture was filtered using fritted glass funnel under vacuum, washed sequentially with de-ionized water and ethanol, and finally air dried.

### Example A22

### Preparation of poly[styrene-co-4-vinylbenzenesulfonic acid-co-1-(4-vinylbenzyl)-piperidine-co-divinyl benzene]

Poly[styrene-co-1-(4-vinylbenzyl)-piperidine-co-divinyl benzene] (7 g) was charged into a 100 mL flask equipped with a magnetic stir bar and condenser. Cold concentrated sulfuric acid (>98% w/w, H₂SO₄, 45 mL) and fuming sulfuric acid (20% free SO₃, 12 mL) were gradually added into the flask under stirring which consequently resulted in the formation of dark-red colored slurry. The slurry was stirred at 95° C. for 8 h. After completion of reaction, the cooled reaction mixture was filtered using fritted glass funnel under vacuum and then washed repeatedly with de-ionized water until the effluent was neutral, as determined by pH paper. The resin beads were finally washed with ethanol and air dried. The chemical functionalization of the polymer with sulfonic acid groups was determined to be 0.72 mmol/g, as determined by titrimetry following the procedure of Example A2.

### Example A23

### Preparation of poly[styrene-co-4-vinylbenzenesulfonic acid-co-1-methyl-1-(4-vinylbenzyl)-piperdin-1-ium chloride-co-divinyl benzene]

Poly (styrene-co-4-(1-piperidino)methylstyrene-co-divinylbenzene) (4 g) was charged into a 100 mL flask equipped with a magnetic stir bar and condenser. Dry dimethylformamide (40 ml) was added into the flask (via cannula under N₂) under stirring to obtain uniform viscous slurry. Iodomethane (1.2 ml) and potassium iodide (10 mg) were then added into the flask. The reaction mixture was stirred at 95° C. for 24 h. After cooling, the reaction mixture was filtered using fritted glass funnel under vacuum and then washed multiple times with dilute HCl solution to ensure complete exchange of F with Cl⁻. The resin was finally washed with de-ionized water until the effluent was neutral, as determined by pH paper. The resin was finally air-dried.

### Example A24

### Preparation of poly[styrene-co-4-(4-vinylbenzyl)-morpholine-co-divinyl benzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (50 ml) was added into the flask (via cannula under N₂) while stirring and consequently, the uniform viscous slurry of polymer resin was obtained. Morpholine (4 g, 45.92 mmol) was then added to the resin slurry and the resulting reaction mixture was heated at 95° C. under continuous stirring for 16 h. After completion of reaction, the reaction mixture was cooled, filtered using fritted glass funnel under vacuum, washed sequentially with de-ionized water and ethanol, and finally air dried.

### Example A25

### Preparation of poly[styrene-co-4-vinylbenzenesulfonic acid-co-4-(4-vinylbenzyl)-morpholine-co-divinyl benzene]

Poly[styrene-co-4-(4-vinylbenzyl)-morpholine-co-divinyl benzene] (10 g) was charged into a 200 mL flask equipped with a magnetic stir bar and condenser. Cold concentrated sulfuric acid (>98% w/w, H₂SO₄, 90 mL) and fuming sulfuric acid (20% free SO₃, 10 mL) were gradually added into the flask while stirring which consequently resulted in the formation of dark-red colored slurry. The slurry was stirred at 95° C. for 8 h. After cooling, the reaction mixture was filtered using fritted glass funnel under vacuum and then washed repeatedly with de-ionized water until the effluent was neutral, as determined by pH paper. The sulfonated resin beads were finally washed with ethanol and air dried. The chemical functionalization of the polymer with sulfonic acid groups was determined to be 0.34 mmol/g, as determined by titrimetry following the procedure of Example A2.

### Example A26

### Preparation of poly[styrene-co-4-vinylbenzenesulfonic acid-co-4-(4-vinylbenzyl)-morpholine-4-oxide-co-divinyl benzene]

Poly[styrene-co-4-vinylbenzenesulfonic acid-co-4-(4-vinylbenzyl)-morpholine-co-divinyl benzene] (6 g) was charged into a 100 mL flask equipped with a magnetic stir bar and condenser. Methanol (60 mL) was then charged into the flask, followed by addition of hydrogen peroxide (30% solution in water, 8.5 mL). The reaction mixture was refluxed under continuous stirring for 8 h. After cooling, the reaction mixture was filtered, washed sequentially with de-ionized water and ethanol, and finally air dried.

### Example A27

### Preparation of poly[styrene-co-4-vinylbenzyl-triethylammonium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (80 ml) was added into the flask (via cannula under N₂) while stirring and consequently the uniform viscous slurry of polymer resin was obtained. Triethylamine (5 mL, 49.41 mmol) was then added to the resin slurry and the resulting reaction mixture was stirred at 95° C. for 18 h. After cooling, the reaction mixture was filtered using fritted glass funnel under vacuum, washed sequentially with de-ionized water and ethanol, and finally air dried. The chemical functionalization of the polymer resin with triethylammonium chloride groups was determined to be 2.61 mmol/g, as determined by titrimetry following the procedure of Example A2.

### Example A28

### Preparation of poly[styrene-co-4-vinylbenzenesulfonic acid-co-triethyl-(4-vinylbenzyl)-ammonium chloride-co-divinylbenzene]

Poly[styrene-co-triethyl-(4-vinylbenzyl)-ammonium chloride-co-divinylbenzene] (6 g) was charged into a 100 mL flask equipped with a magnetic stir bar and condenser. Cold concentrated sulfuric acid (>98% w/w, H₂SO₄, 60 mL) was gradually added into the flask under stirring which consequently resulted in the formation of dark-red colored uniform slurry of resin. The slurry was stirred at 95-100° C. for 8 h. After cooling, the reaction mixture was filtered using fritted glass funnel under vacuum and then washed repeatedly with de-ionized water until the effluent was neutral, as determined by pH paper. The sulfonated resin beads were finally washed with ethanol and air dried. The chemical functionalization of the polymer with sulfonic acid groups was determined to be 0.31 mmol/g, as determined by titrimetry following the procedure of Example A2.

### Example A29

### Preparation of poly[styrene-co-4-vinylbenzenesulfonic acid-co-vinylbenzylchloride-co-divinylbenzene]

Poly(styrene-co-vinylbenzyl chloride-co-divinylbenzene) (6 g) was charged into a 100 mL flask equipped with a magnetic stir bar and condenser. Fuming sulfuric acid (20% free SO₃, 25 mL) was gradually added into the flask under stirring which consequently resulted in the formation of dark-red colored slurry. The slurry was stirred at 90° C. for 5 h. After cooling, the reaction mixture was filtered using fritted glass funnel under vacuum, washed sequentially with de-ionized water and ethanol, and finally air dried. The chemical functionalization of the polymer with sulfonic acid groups was determined to be 0.34 mmol/g, as determined by titrimetry following the procedure of Example A2.

### Example A30

### Preparation of poly[styrene-co-4-vinylbenzenesulfonic acid-co-3-methyl-1-(4-vinylbenzyl)-3H-imidazol-1-ium chloride-co-divinylbenzene]

Poly[styrene-co-4-vinylbenzenesulfonic acid-co-vinylbenzylchloride-co-divinylbenzene] (5 g) was charged into a 100 mL flask equipped with a magnetic stir bar and condenser. Dry dimethylformamide (20 ml) was added into the flask (via cannula under N₂) while stirring and the uniform viscous slurry of polymer resin was obtained. 1-Methylimidazole (3 mL, 49.41 mmol) was then added to the resin slurry and the resulting reaction mixture was stirred at 95° C. for 18 h. After cooling, reaction mixture was filtered using fritted glass funnel under vacuum and then washed repeatedly with de-ionized water. The resin beads were finally washed with ethanol and air dried. The chemical functionalization of the polymer with sulfonic acid group and methylimidiazolium chloride groups was determined to be 0.23 mmol/g and 2.63 mmol/g, respectively, as determined by titrimetry following the procedure of Example A2.

### Example A31

### Preparation of poly[styrene-co-3-methyl-1-(4-vinylbenzyl)-3H-imidazol-1-ium chloride-co-4-boronyl-1-(4-vinylbenzyl)-pyridinium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (80 ml) was added into the flask (via cannula under N₂) while stirring and consequently the uniform viscous slurry of polymer resin was obtained. 4-Pyridyl-boronic acid (1.8 g, 14.6 mmol) was then added to the resin slurry and the resulting reaction mixture was stirred at 95° C. for 2 days. 1-Methylimidazole (3 mL, 49.41 mmol) was then added to the reaction mixture and stirred further at 95° C. for 1 day. After cooling to room temperature, the reaction mixture was filtered using fritted glass funnel under vacuum, washed sequentially with de-ionized water and ethanol, and finally air dried. The chemical functionalization of the polymer with boronic acid group was determined to be 0.28 mmol/g respectively, as determined by titrimetry following the procedure of Example A2.

### Example A32

### Preparation of poly[styrene-co-3-methyl-1-(4-vinylbenzyl)-3H-imidazol-1-ium chloride-co-1-(4-vinylphenyl)methylphosphonic acid-co-divinylbenzene]

Poly[styrene-co-3-methyl-1-(4-vinylbenzyl)-3H-imidazol-1-ium chloride-co-divinylbenzene](Cl⁻ density=˜2.73 mmol/g, 5 g) was charged into a 100 mL flask equipped with a magnetic stir bar and condenser. Triethylphosphite (70 ml) was added into the flask and the resulting suspension was stirred at 120° C. for 2 days. The reaction mixture was filtered using fritted glass funnel and the resin beads were washed repeatedly with de-ionized water and ethanol. These resin beads were then suspended in concentrated HCl (80 ml) and refluxed at 115° C. under continuous stirring for 24 h. After cooling to room temperature, the reaction mixture was filtered using fritted glass funnel under vacuum and then washed repeatedly with de-ionized water. The resin beads were finally washed with ethanol and air dried. The chemical functionalization of the polymer with phosphonic acid group and methylimidiazolium chloride groups was determined to be 0.11 mmol/g and 2.81 mmol/g, respectively, as determined by titrimetry following the procedure of Example A2.

### Example A33

### Preparation of poly[styrene-co-4-vinylbenzenesulfonic acid-co-vinylbenzylchloride-co-vinyl-2-pyridine-co-divinylbenzene]

Poly (styrene-co-vinylbenzylchloride-co-vinyl-2-pyridine-co-divinylbenzene) (5 g) was charged into a 100 mL flask equipped with a magnetic stir bar and condenser. Cold concentrated sulfuric acid (>98% w/w, H₂SO₄, 80 mL) was gradually added into the flask under stirring which consequently resulted in the formation of dark-red colored slurry. The slurry was stirred at 95° C. for 8 h. After cooling to room temperature, the reaction mixture was filtered using fritted glass funnel under vacuum, washed repeatedly with de-ionized water until the effluent was neutral, as determined by pH paper. The sulfonated beads were finally washed with ethanol and air dried. The chemical functionalization of the polymer with sulfonic acid groups was determined to be 3.49 mmol/g, as determined by titrimetry following the procedure of Example A2.

### Example A34

### Preparation of poly[styrene-co-4-vinylbenzenesulfonic acid-co-vinylbenzylchloride-co-1-methyl-2-vinyl-pyridinium chloride-co-divinylbenzene]

Poly[styrene-co-4-vinylbenzenesulfonic acid-co-vinylbenzylchloride-co-vinyl-2-pyridine-co-divinylbenzene] (4 g) was charged into a 100 mL flask equipped with a magnetic stir bar and condenser. Dry dimethylformamide (80 ml) was added into the flask (via cannula under N₂) under stirring to obtain uniform viscous slurry. Iodomethane (1.9 ml) was then gradually added into the flask followed by addition of potassium iodide (10 mg). The reaction mixture was stirred at 95° C. for 24 h. After cooling to room temperature, the cooled reaction mixture was filtered using fritted glass funnel under vacuum and then washed multiple times with dilute HCl solution to ensure complete exchange of I⁻ with Cl⁻. The resin beads were finally washed with de-ionized water until the effluent was neutral, as determined by pH paper and then air-dried.

### Example A35

### Preparation of poly[styrene-co-4-vinylbenzenesulfonic acid-co-4-(4-vinylbenzyl)-morpholine-4-oxide-co-divinyl benzene]

Poly[styrene-co-4-(4-vinylbenzyl)-morpholine-4-oxide-co-divinyl benzene] (3 g) was charged into a 100 mL flask equipped with a magnetic stir bar and condenser. Cold concentrated sulfuric acid (>98% w/w, H₂SO₄, 45 mL) was gradually added into the flask under stirring which consequently resulted in the formation of dark-red colored slurry. The slurry was stirred at 95° C. for 8 h. After cooling to room temperature, the reaction mixture was filtered using fritted glass funnel under vacuum, washed repeatedly with de-ionized water until the effluent was neutral, as determined by pH paper. The sulfonated beads were finally washed with ethanol and air dried.

### Example A36

### Preparation of poly[styrene-co-4-vinylphenylphosphonic acid-co-3-methyl-1-(4-vinylbenzyl)-3H-imidazol-1-ium chloride-co-divinylbenzene]

Poly[styrene-co-3-methyl-1-(4-vinylbenzyl)-3H-imidazol-1-iumchloride-co-divinylbenzene] (Cl⁻ density=˜2.73 mmol/g, 5 g) was charged into a 100 mL flask equipped with a magnetic stir bar and condenser. Diethylphosphite (30 ml) and t-butylperoxide (3.2 ml) were added into the flask and the resulting suspension was stirred at 120° C. for 2 days. The reaction mixture was filtered using fritted glass funnel and the resin beads were washed repeatedly with de-ionized water and ethanol. These resin beads were then suspended in concentrated HCl (80 ml) and refluxed at 115° C. under continuous stirring for 2 days. After cooling to room temperature, the reaction mixture was filtered using fritted glass funnel under vacuum and then washed repeatedly with de-ionized water. The resin beads were finally washed with ethanol and air dried. The chemical functionalization of the polymer with aromatic phosphonic acid group was determined to be 0.15 mmol/g, as determined by titrimetry following the procedure of Example A2.

### Example A37

### Preparation of poly[styrene-co-3-carboxymethyl-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dimethylformamide (50 ml) was added into the flask and stirred to form a slurry of resin. Imidazole (2.8 g, 41.13 mmol) was then added to the resin slurry and stirred at 80° C. for 8 h. The reaction mixture was then cooled to 40° C. and t-butoxide (1.8 g) was added into the reaction mixture and stirred for 1 h. Bromoethylacetate (4 ml) was then added to and the reaction mixture was stirred at 80° C. for 6 h. After cooling to room temperature, the reaction mixture was filtered using fritted glass funnel under vacuum and then washed repeatedly with de-ionized water. The washed resin beads were suspended in the ethanolic sodium hydroxide solution and refluxed overnight. The resin beads were filtered and successively washed with deionized water multiple times and ethanol, and finally air dried. The chemical functionalization of the polymer with carboxylic acid group was determined to be 0.09 mmol/g, as determined by titrimetry following the procedure of Example A2.

### Example A38

### Preparation of poly[styrene-co-5-(4-vinylbenzylamino)-isophthalic acid-co-3-methyl-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (80 ml) was added into the flask (via cannula under N₂) while stirring and consequently the uniform viscous slurry of polymer resin was obtained. Dimethyl aminoisophthalate (3.0 g, 14.3 mmol) was then added to the resin slurry and the resulting reaction mixture was stirred at 95° C. for 16 h. 1-Methylimidazole (2.3 mL, 28.4 mmol) was then added to the reaction mixture and stirred further at 95° C. for 1 day. After cooling to room temperature, the reaction mixture was filtered using fritted glass funnel under vacuum, washed sequentially with de-ionized water and ethanol. The washed resin beads were suspended in the ethanolic sodium hydroxide solution and refluxed overnight. The resin beads were filtered and successively washed with deionized water multiple times and ethanol, and finally air dried. The chemical functionalization of the polymer with carboxylic acid group was determined to be 0.16 mmol/g, as determined by titrimetry following the procedure of Example A2.

### Example A39

### Preparation of poly[styrene-co-(4-vinylbenzylamino)-acetic acid-co-3-methyl-1-(4-vinylbenzyl)-3H-imidazol-1-ium chloride-co-divinylbenzene]

Poly(styrene-co-vinylbenzylchloride-co-divinylbenzene) (Cl⁻ density=˜4.0 mmol/g, 10 g, 40 mmol) was charged into a 100 mL flask equipped with a magnetic stir bar and condenser. Dry dimethylformamide (80 ml) was added into the flask (via cannula under N₂) while stirring and consequently the uniform viscous slurry of polymer resin was obtained. Glycine (1.2 g, 15.9 mmol) was then added to the resin slurry and the resulting reaction mixture was stirred at 95° C. for 2 days. 1-Methylimidazole (2.3 mL, 28.4 mmol) was then added to the reaction mixture and stirred further at 95° C. for 12 hours. After cooling to room temperature, the reaction mixture was filtered using fritted glass funnel under vacuum, washed sequentially with de-ionized water and ethanol, and finally air dried. The chemical functionalization of the polymer with carboxylic acid group was determined to be 0.05 mmol/g, as determined by titrimetry following the procedure of Example A2.

### Example A40

### Preparation of poly[styrene-co-(1-vinyl-1H-imidazole)-co-divinylbenzene]

To a 500 mL round bottom flask (RBF) containing a stirred solution of 1.00 g of poly(vinylalcohol) in 250.0 mL of deionized H₂O at 0° C. is gradually added a solution containing 35 g (371 mmol) of 1-vinylimidazole, 10 g (96 mmol) of styrene, 1 g (7.7 mmol) of divinylbenzene (DVB) and 1.5 g (9.1 mmol) of azobisisobutyronitrile (AIBN) in 150 mL of a 1:1 (by volume) mixture of benzene/tetrahydrofuran (THF) at 0° C. After 2 hours of stirring at 0° C. to homogenize the mixture, the reaction flask is transferred to an oil bath to increase the reaction temperature to 75° C., and the mixture is stirred vigorously for 24 hours. The resulting polymer is vacuum filtered using a fritted-glass funnel, washed repeatedly with 20% (by volume) methanol in water, THF, and MeOH, and then dried overnight at 50° C. under reduced pressure.

### Example A41

### Preparation of poly(styrene-co-vinylbenzylmethylimidazolium chloride-co-vinylbenzylmethylmorpholinium chloride-co-vinylbenzyltriphenylphosphonium chloride-co-divinylbenzene)

1-methylimidazole (4.61 g, 56.2 mmol), 4-methylmorpholine (5.65 g, 56.2 mmol), and triphenylphosphine (14.65, 55.9 mmol) were charged into a 500 mL flask equipped with a magnetic stir bar and a condenser. Acetone (100 ml) was added into the flask and mixture was stirred at 50° C. for 10 min. Poly(styrene-co-vinylbenzylchloride-co-divinylbenzene) (1% DVB, Cl⁻ density=4.18 mmol/g dry resin, 40.22 g, 168 mmol) was charged into the flask while stirring until a uniform polymer suspension was obtained. The resulting reaction mixture was refluxed for 24 h. After cooling, the reaction mixture was filtered using a fritted glass funnel under vacuum, washed sequentially with acetone and ethyl acetate, and dried overnight at 70° C. The chemical functionalization of the polymer resin with chloride groups was determined to be 2.61 mmol/g dry resin via titrimetry.

### Example A42

### Preparation of sulfonated poly(styrene-co-vinylbenzylmethylimidazolium bisulfate-co-vinylbenzylmethylmorpholinium bisulfate-co-vinylbenzyltriphenyl phosphonium bisulfate-co-divinylbenzene)

Poly(styrene-co-vinylbenzylmethylimidazolium chloride-co-vinylbenzylmethylmorpholinium chloride-co-vinylbenzyltriphenylphosphonium chloride-co-divinylbenzene) (35.02 g) was charged into a 500 mL flask equipped with a magnetic stir bar and condenser. Fuming sulfuric acid (20% free SO₃, 175 mL) was gradually added into the flask and stirred to form dark-red resin suspension. The mixture was stirred overnight at 90° C. After cooling to room temperature, the reaction mixture was filtered using fritted glass funnel under vacuum and then washed repeatedly with de-ionized water until the effluent was neutral, as determined by pH paper. The sulfonated polymer resin was air dried to a final moisture content of 56% g H₂O/g wet polymer. The chemical functionalization of the polymer resin with sulfonic acid groups was determined to be 3.65 mmol/g dry resin.

### Example A43

### Preparation of poly(styrene-co-vinylbenzylmethylimidazolium chloride-co-vinylbenzylmethylmorpholinium chloride-co-vinylbenzyltriphenylphosphonium chloride-co-divinylbenzene)

1-methylimidazole (7.02 g, 85.5 mmol), 4-methylmorpholine (4.37 g, 43.2 mmol) and triphenylphosphine (11.09, 42.3 mmol) were charged into a 500 mL flask equipped with a magnetic stir bar and condenser. Acetone (100 ml) was added into the flask and mixture was stirred at 50° C. for 10 min. Poly(styrene-co-vinylbenzylchloride-co-divinylbenzene) (1% DVB, Cl⁻ density=4.18 mmol/g dry resin, 40.38 g, 169 mmol) was charged into flask while stirring until a uniform suspension was obtained. The resulting reaction mixture was refluxed for 18 h. After cooling, the reaction mixture was filtered using fritted glass funnel under vacuum, washed sequentially with acetone and ethyl acetate, and dried at 70° C. overnight. The chemical functionalization of the polymer resin with chloride groups was determined to be 2.36 mmol/g dry resin dry resin via titrimetry.

### Example A44

### Preparation of sulfonated poly(styrene-co-vinylbenzylmethylimidazolium bisulfate-co-vinylbenzylmethylmorpholinium bisulfate-co-vinylbenzyltriphenyl phosphonium bisulfate-co-divinylbenzene)

Poly(styrene-co-vinylbenzylmethylimidazolium chloride-co-vinylbenzylmethylmorpholinium chloride-co-vinylbenzyltriphenylphosphonium chloride-co-divinylbenzene) (35.12 g) was charged into a 500 mL flask equipped with a magnetic stir bar and condenser. Fuming sulfuric acid (20% free SO₃, 175 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were finally air dried. The chemical functionalization of the polymer resin with sulfonic acid groups was determined to be 4.38 mmol/g dry resin.

### Example A45

### Preparation of poly(styrene-co-vinylbenzylmethylmorpholinium chloride-co-vinylbenzyltriphenylphosphonium chloride-co-divinylbenzene)

4-methylmorpholine (8.65 g, 85.5 mmol) and triphenylphosphine (22.41, 85.3 mmol) were charged into a 500 mL flask equipped with a magnetic stir bar and condenser. Acetone (100 ml) was added into the flask and mixture was stirred at 50° C. for 10 min. Poly(styrene-co-vinylbenzylchloride-co-divinylbenzene) (1% DVB, Cl⁻ density=4.18 mmol/g dry resin, 40.12 g, 167 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight. The chemical functionalization of the polymer resin with chloride groups was determined to be 2.22 mmol/g dry resin via titrimetry.

### Example A46

### Preparation of sulfonated poly(styrene-co-vinylbenzylmethylmorpholinium bisulfate-co-vinylbenzyltriphenylphosphonium bisulfate-co-divinylbenzene)

Poly(styrene-co-vinylbenzylmethylimidazolium chloride-co-vinylbenzylmethylmorpholinium chloride-co-vinylbenzyltriphenylphosphonium chloride-co-divinylbenzene) (35.08 g) was charged into a 500 mL flask equipped with a magnetic stir bar and condenser. Fuming sulfuric acid (20% free SO₃, 175 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 52% g H₂O/g wet resin. The chemical functionalization of the polymer resin with sulfonic acid groups was determined to be 4.24 mmol/g dry resin.

### Example A47

### Preparation of phenol-formaldehyde resin

Phenol (12.87 g, 136.8 mmol) was dispensed into a 100 mL round bottom flask (RBF) equipped with a stir bar and condenser. De-ionized water (10 g) was charged into the flask. 37% Formalin solution (9.24 g, 110 mmol) and oxalic acid (75 mg) were added. The resulting reaction mixture was refluxed for 30 min. Additional oxalic acid (75 mg) was then added and refluxing was continued for another 1 hour. Chunk of solid resin was formed, which was ground to a coarse powder using a mortar and pestle. The resin was repeatedly washed with water and methanol and then dried at 70° C. overnight.

### Example A48

### Preparation of Chloromethylated Phenol-Formaldehyde Resin

Phenol-formaldehyde resin (5.23 g, 44 mmol) was dispensed into a 100 mL three neck round bottom flask (RBF) equipped with a stir bar, condenser and nitrogen line. Anhydrous dichloroethane (DCE, 20 ml) was then charged into the flask. To ice-cooled suspension of resin in DCE, zinc chloride (6.83 g, 50 mmol) was added. Chloromethyl methyl ether (4.0 ml, 51 mmol) was then added dropwise into the reaction. The mixture was warmed to room temperature and stirred at 50° C. for 6 h. The product resin was recovered by vacuum filtration and washed sequentially with water, acetone and dichloromethane. The washed resin was dried at 40° C. overnight.

### Example A49

### Preparation of Triphenylphosphine Functionalized Phenol-Formaldehyde Resin

Triphenylphosphine (10.12, 38.61 mmol) were charged into a 100 mL flask equipped with a magnetic stir bar and condenser. Acetone (30 ml) was added into the flask and mixture was stirred at 50° C. for 10 min. Chloromethylated phenol-formaldehyde resin (4.61 g, 38.03 mmol) was charged into flask while stirring. The resulting reaction mixture was refluxed for 24 h. After cooling, the reaction mixture was filtered using fritted glass funnel under vacuum, washed sequentially with acetone and ethyl acetate, and dried at 70° C. overnight.

### Example A50

### Preparation of Sulfonated Triphenylphosphine-Functionalized Phenol-Formaldehyde Resin

Triphenylphosphine-functionalized phenol-formaldeyde resin (5.12 g, 13.4 mmol) was charged into a 100 mL flask equipped with a magnetic stir bar and condenser. Fuming sulfuric acid (20% free SO₃, 25 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated resin was dried under air to a final moisture content of 49% g H₂O/g wet resin. The chemical functionalization of the polymer resin with sulfonic acid groups was determined to be 3.85 mmol/g dry resin.

### Example A51

### Preparation of poly(styrene-co-vinylimidazole-co-divinylbenzene)

De-ionized water (75 mL) was charged into flask into a 500 mL three neck round bottom flask equipped with a mechanical stirrer, condenser and N₂ line. Sodium chloride (1.18 g) and carboxymethylcellulose (0.61 g) were charged into the flask and stirred for 5 min. The solution of vinylimidazole (3.9 mL, 42.62 mmol), styrene (4.9 mL, 42.33 mmol) and divinylbenzene (0.9 mL, 4.0 mmol) in iso-octanol (25 mL) was charged into flask. The resulting emulsion was stirred at 500 rpm at room temperature for 1 h. Benzoyl peroxide (75%, 1.205 g) was added, and temperature was raised to 80° C. The reaction mixture was heated for 8 h at 80° C. with stirring rate of 500 rpm. The polymer product was recovered by vacuum filtration and washed with water and acetone multiple times. The isolated polymer was purified by soxhlet extraction with water and acetone. The resin was dried at 40° C. overnight.

### Example A52

### Preparation of poly(styrene-co-vinylmethylimidazolium iodide-co-divinylbenzene)

Poly(styrene-co-vinylimidazole-co-divinylbenzene) (3.49 g, 39 mmol) was dispensed into a 100 mL three neck round bottom flask (RBF) equipped with a stir bar, condenser and nitrogen line. Anhydrous tetrahydrofuran (20 ml) was then charged into the flask. To ice-cooled suspension of resin in tetrahydrofuran, potassium t-butoxide (5.62 g, 50 mmol) was added and stirred for 30 min. Iodomethane (3.2 ml, 51 mmol) was then added dropwise into the reaction. The mixture was warmed to room temperature and stirred at 50° C. for 6 h. The product resin was recovered by vacuum filtration and washed sequentially with water, acetone and dichloromethane. The washed resin was dried at 40° C. overnight.

### Example A53

### Preparation of sulfonated poly(styrene-co-vinylmethylimidazolium bisulfate-co-divinylbenzene)

Poly(styrene-co-vinylmethylimidazolium iodide-co-divinylbenzene) (3.89 g, 27.8 mmol) was charged into a 100 mL flask equipped with a magnetic stir bar and condenser. Fuming sulfuric acid (20% free SO₃, 20 mL) was gradually added into the flask and stirred to form dark-red colored slurry. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated polymer was dried under air to a final moisture content of 51% g H₂O/g wet resin.

### Example A54

### Preparation of poly(styrene-co-vinylbenzyltriphenylphosphonium chloride-co-divinylbenzene)

To a 250 mL flask equipped with a magnetic stir bar and condenser was charged triphenylphosphine (38.44 g, 145.1 mmol). Acetone (50 mL) was added into the flask and mixture was stirred at 50° C. for 10 min. Poly(styrene-co-vinylbenzylchloride-co-divinylbenzene) (8% DVB, Cl⁻ density=4.0 mmol/g dry resin, 30.12 g, 115.6 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight. The chemical functionalization of the polymer resin with triphenylphosphonium chloride groups was determined to be 1.94 mmol/g dry resin via titrimetry.

### Example A55

### Preparation of sulfonated poly(styrene-co-vinylbenzyltriphenyl phosphonium bisulfate-co-divinylbenzene)

Poly(styrene-co-vinylbenzyltriphenylphosphonium chloride-co-divinylbenzene) (40.12 g) was charged into a 500 mL flask equipped with a magnetic stir bar and condenser. Fuming sulfuric acid (20% free SO₃, 160 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 54% g H₂O/g wet resin. The chemical functionalization of the polymer resin with sulfonic acid groups was determined to be 4.39 mmol/g dry resin.

### Example A56

### Preparation of poly(styrene-co-vinylbenzyltriphenylphosphonium chloride-co-divinylbenzene

To a 250 mL flask equipped with a magnetic stir bar and condenser was charged triphenylphosphine (50.22 g, 189.6 mmol). Acetone (50 mL) was added into the flask and mixture was stirred at 50° C. for 10 min. Poly(styrene-co-vinylbenzylchloride-co-divinylbenzene) (4% DVB, Cl⁻ density=5.2 mmol/g dry resin, 30.06 g, 152.08 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight. The chemical functionalization of the polymer resin with triphenylphosphonium chloride groups was determined to be 2.00 mmol/g dry resin via titrimetry.

### Example A57

### Preparation of sulfonated poly(styrene-co-vinylbenzyltriphenyl phosphonium bisulfate-co-divinylbenzene)

Poly(styrene-co-vinylbenzyltriphenylphosphonium chloride-co-divinylbenzene) (40.04 g,) was charged into a 500 mL flask equipped with a magnetic stir bar and condenser. Fuming sulfuric acid (20% free SO₃, 160 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 47% g H₂O/g wet resin. The chemical functionalization of the polymer resin with sulfonic acid groups was determined to be 4.36 mmol/g dry resin.

### Example A58

### Preparation of poly(styrene-co-vinylbenzylmethylimidazolium chloride-co-divinylbenzene)

To a 250 mL flask equipped with a magnetic stir bar and condenser was charged 1-methylimidazole (18 mL, 223.5 mmol). Acetone (75 mL) was added into the flask and mixture was stirred at 50° C. for 10 min. Poly(styrene-co-vinylbenzylchloride-co-divinylbenzene) (8% DVB, Cl⁻ density=4.0 mmol/g dry resin, 40.06, 153.7 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight. The chemical functionalization of the polymer resin with methylimidazolium chloride groups was determined to be 3.54 mmol/g dry resin via titrimetry.

### Example A59

### Preparation of sulfonated poly(styrene-co-vinylbenzylmethylimidazolium bisulfate-co-divinylbenzene)

Poly(styrene-co-vinylbenzylmethylimidazolium chloride-co-divinylbenzene) (30.08 g) was charged into a 500 mL flask equipped with a magnetic stir bar and condenser. Fuming sulfuric acid (20% free SO₃, 120 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 50% g H₂O/g wet resin. The chemical functionalization of the polymer resin with sulfonic acid groups was determined to be 2.87 mmol/g dry resin.

### Example A60

### Preparation of poly(styrene-co-vinylbenzylmethylimidazolium chloride-co-divinylbenzene)

To a 250 mL flask equipped with a magnetic stir bar and condenser was charged 1-methylimidazole (20 mL, 248.4 mmol). Acetone (75 mL) was added into the flask and mixture was stirred at 50° C. for 10 min. Poly(styrene-co-vinylbenzylchloride-co-divinylbenzene) (4% DVB, Cl⁻ density=5.2 mmol/g dry resin, 40.08, 203.8 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight. The chemical functionalization of the polymer resin with methylimidazolium chloride groups was determined to be 3.39 mmol/g dry resin via titrimetry.

### Example A61

### Preparation of sulfonated poly(styrene-co-vinylbenzylmethylimidazolium bisulfate-co-divinylbenzene)

Poly(styrene-co-vinylbenzylmethylimidazolium chloride-co-divinylbenzene) (30.14 g) was charged into a 500 mL flask equipped with a magnetic stir bar and condenser. Fuming sulfuric acid (20% free SO₃, 120 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 55% g H₂O/g wet resin. The chemical functionalization of the polymer resin with sulfonic acid groups was determined to be 2.78 mmol/g dry resin.

### Example A62

### Preparation of poly(styrene-co-vinylbenzyltriphenylphosphonium chloride-co-divinylbenzene)

To a 250 mL flask equipped with a magnetic stir bar and condenser was charged triphenylphosphine (44.32 g, 163.9 mmol). Acetone (50 mL) was added into the flask and mixture was stirred at 50° C. for 10 min. Poly(styrene-co-vinylbenzylchloride-co-divinylbenzene) (13% DVB macroporous resin, Cl⁻ density=4.14 mmol/g dry resin, 30.12 g, 115.6 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight.

### Example A63

### Preparation of sulfonated poly(styrene-co-vinylbenzyltriphenyl phosphonium bisulfate-co-divinylbenzene)

Poly(styrene-co-vinylbenzyltriphenylphosphonium chloride-co-divinylbenzene) (30.22 g) was charged into a 500 mL flask equipped with a magnetic stir bar and condenser. Fuming sulfuric acid (20% free SO₃, 90 mL) was gradually added into the flask and stirred to form dark-red colored slurry of resin. The slurry was stirred at 90° C. for 1 hour. After cooling, the reaction mixture was filtered using fritted glass funnel under vacuum and then washed repeatedly with de-ionized water until the effluent was neutral, as determined by pH paper. The sulfonated beads were dried under air to a final moisture content of 46% g H₂O/g wet resin. The chemical functionalization of the polymer resin with sulfonic acid groups was determined to be 2.82 mmol/g dry resin.

### Example A64

### Preparation of poly(styrene-co-vinylbenzyltriphenylphosphonium chloride-co-divinylbenzene)

To a 250 mL flask equipped with a magnetic stir bar and condenser was charged triphenylphosphine (55.02 g, 207.7 mmol). Acetone (50 mL) was added into the flask and mixture was stirred at 50° C. for 10 min. Poly(styrene-co-vinylbenzylchloride-co-divinylbenzene) (6.5% DVB macroporous resin, Cl⁻ density=5.30 mmol/g dry resin, 30.12 g, 157.4 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight.

### Example A65

### Preparation of sulfonated poly(styrene-co-vinylbenzyltriphenyl phosphonium bisulfate-co-divinylbenzene)

Poly(styrene-co-vinylbenzyltriphenylphosphonium chloride-co-divinylbenzene) (30.12 g) was charged into a 500 mL flask equipped with a magnetic stir bar and condenser. Fuming sulfuric acid (20% free SO₃, 90 mL) was gradually added into the flask and stirred to form dark-red colored slurry of resin. The slurry was stirred at 90° C. for 1 hour. After cooling, the reaction mixture was filtered using fritted glass funnel under vacuum and then washed repeatedly with de-ionized water until the effluent was neutral, as determined by pH paper. The sulfonated beads were dried under air to a final moisture content of 49% g H₂O/g wet resin. The chemical functionalization of the polymer resin with sulfonic acid groups was determined to be 2.82 mmol/g dry resin.

### Example A66

### Preparation of poly(styrene-co-vinylbenzyltriphenylphosphonium chloride-co-divinylbenzene)

To a 250 mL flask equipped with a magnetic stir bar and condenser was charged triphenylphosphine (38.42 g, 145.0 mmol). Acetone (50 mL) was added into the flask and mixture was stirred at 50° C. for 10 min. Poly(styrene-co-vinylbenzylchloride-co-divinylbenzene) (4% DVB, Cl⁻ density=4.10 mmol/g dry resin, 30.12 g, 115.4 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight.

### Example A67

### Preparation of sulfonated poly(styrene-co-vinylbenzyltriphenylphosphonium bisulfate-co-divinylbenzene)

Poly(styrene-co-vinylbenzyltriphenylphosphonium chloride-co-divinylbenzene) (30.18 g) was charged into a 500 mL flask equipped with a magnetic stir bar and condenser. Fuming sulfuric acid (20% free SO₃, 120 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 59% g H₂O/g wet resin. The chemical functionalization of the polymer resin with sulfonic acid groups was determined to be 3.03 mmol/g dry resin.

### Example A68

### Preparation of poly(styrene-co-vinylbenzyltriphenylphosphonium chloride-co-divinylbenzene)

To a 500 mL flask equipped with a magnetic stir bar and condenser was charged triphenylphosphine (44.22 g, 166.9 mmol). Acetone (70 mL) was added into the flask and mixture was stirred at 50° C. for 10 min. Poly(styrene-co-vinylbenzylchloride-co-divinylbenzene) (4% DVB, Cl⁻ density=3.9 mmol/g dry resin, 35.08 g, 130.4 mmol) was charged into flask while stirring until a uniform suspension was obtained. The resulting reaction mixture was refluxed for 24 h. After cooling, the reaction mixture was filtered using fritted glass funnel under vacuum, washed sequentially with acetone and ethyl acetate, and dried at 70° C. overnight.

### Example A69

### Preparation of sulfonated poly(styrene-co-vinylbenzyltriphenyl phosphonium bisulfate-co-divinylbenzene)

Poly(styrene-co-vinylbenzyltriphenylphosphonium chloride-co-divinylbenzene) (30.42 g) was charged into a 500 mL flask equipped with a magnetic stir bar and condenser. Fuming sulfuric acid (20% free SO₃, 120 mL) was gradually added into the flask and stirred to form dark-red colored slurry of resin. The slurry was stirred at 90° C. overnight. After cooling, the reaction mixture was filtered using fritted glass funnel under vacuum and then washed repeatedly with de-ionized water until the effluent was neutral, as determined by pH paper. The sulfonated beads were dried under air to a final moisture content of 57% g H₂O/g wet resin. The chemical functionalization of the polymer resin with sulfonic acid groups was determined to be 3.04 mmol/g dry resin.

### Example A70

### Preparation of poly(butyl-vinylimidazolium chloride-co-butylimidazolium chloride-co-styrene)

To a 500 mL flask equipped with a mechanical stirrer and reflux condenser is added 250 mL of acetone, 10 g of imidzole, 14 g of vinylimidazole, 15 g of styrene, 30 g of dichlorobutane and 1 g of azobisisobutyronitrile (AIBN). The solution is stirred under reflux conditions for 12 hours to produce a solid mass of polymer. The solid polymer is removed from the flask, washed repeatedly with acetone, and ground to a coarse powder using a mortar and pestle to yield the product.

### Example A71

### Preparation of sulfonated poly(butyl-vinylimidazolium bisulfate-co-butylimidazolium bisulfate-co-styrene)

Poly(butyl-vinylimidazolium chloride-co-butylimidazolium chloride-co-styrene) 30.42 g) is charged into a 500 mL flask equipped with a mechanical stirrer. Fuming sulfuric acid (20% free SO₃, 120 mL) is gradually added into the flask until the polymer is fully suspended. The resulting slurry is stirred at 90° C. for 5 hours. After cooling, the reaction mixture is filtered using fritted glass funnel under vacuum and then washed repeatedly with de-ionized water until the effluent is neutral, as determined by pH paper.

### Preparation of Solid-Supported Acid Catalysts

### Example B1a

### Preparation of the Carbon Support from Populus tremuloides

A carbon-containing starting material was obtained by milling 1.0 kg of commercially-sourced hardwood chips (input moisture content of 15% g H₂O/g wood; carbon content 45% g carbon/g wood) from the Aspen species Populus tremuloides using a 1 horsepower (HP) laboratory rotating knife mill equipped with a 2.0 mm output screen. The output hardwood milling-dust was dried at 70° C. to a moisture content below 5% g H₂O/g wood. 500 g of the resulting dry wood was charged into a 5 L glass reaction vessel equipped with an electric heating jacket, a nitrogen input line, an exhaust line directed to a scrubber with a water bubbler apparatus, and top and bottom thermocouples accurate to ±0.5° C. The atmosphere of the charged reaction vessel was purged with nitrogen for 10 minutes, after which the nitrogen flow was reduced to the minimum required to drive nitrogen through the scrubber apparatus and maintained at that minimum flow rate during the reaction. The temperature was increased gradually over 30 minutes to 350° C., maintained for 4.0 hours, and then decreased to room temperature over a 30-minute period. 175 g of a brown-black material was recovered from the reactor vessel. The product was ground gently into a coarse powder to yield a carbon support material.

### Example B1b

### Preparation of the Carbon Support from Coconut Shell

A carbon-containing starting material is obtained by milling 1.0 kg of commercially-sourced coconut shell chips (input moisture content of 10% g H₂O/g wood; carbon content 50% g carbon/g shells) using a 1 horsepower (HP) laboratory rotating knife mill equipped with a 2.0 mm output screen. The output milling-dust is dried at 70° C. to a moisture content below 5% g H₂O/g material. 500 g of the resulting dry material is charged into a 5 L glass reaction vessel equipped with an electric heating jacket, a nitrogen input line, an exhaust line directed to a scrubber with a water bubbler apparatus, and top and bottom thermocouples that are accurate to 0.5° C. The atmosphere of the charged reaction vessel is purged with nitrogen for 10 minutes, after which the nitrogen flow is reduced to the minimum required to drive nitrogen through the scrubber apparatus and maintained through the reaction. The temperature is increased gradually over 30 minutes to 350° C., maintained for 4.0 hours, and then decreased to room temperature over a 30-minute period. The powder recovered from the reactor vessel is ground gently into a coarse powder to yield the carbon support material.

### Example B1c

### Preparation of the Carbon Support from Shrimp Shell

A carbon-containing starting material was obtained by milling 100 g of commercially-sourced shrimp shells (input moisture content of 10% g H₂O/g wood; carbon content 40% g carbon/g shells and 5% g nitrogen/g shells) using a 1 horsepower (HP) laboratory rotating knife mill equipped with a 2.0 mm output screen. The output milling-dust was dried at 70° C. to a moisture content below 5% g H₂O/g material. 70 g of the resulting dry material was charged into a 1 L glass reaction vessel equipped with an electric heating jacket, a nitrogen input line, an exhaust line directed to a scrubber with a water bubbler apparatus, and top and bottom thermocouples that are accurate to 0.5° C. The atmosphere of the charged reaction vessel was purged with nitrogen for 10 minutes, after which the nitrogen flow was reduced to the minimum required to drive nitrogen through the scrubber apparatus and maintained through the reaction. The temperature was increased gradually over 30 minutes to 350° C., maintained for 4.0 hours, and then decreased to room temperature over a 30-minute period. The powder recovered from the reactor vessel was ground gently into a coarse powder to yield 35.2 g of the carbon support material.

### Example B1d

### Preparation of the Carbon Support from Chitosan

A carbon-containing starting material was obtained by milling 1.0 kg of commercially-available chitosan (input moisture content of 2% g H₂O/g chitosan; carbon content 40% g carbon/g shells and 8% g nitrogen/g shells) using a 1 horsepower (HP) laboratory rotating knife mill equipped with a 2.0 mm output screen. The output milling-dust was dried at 70° C. to a moisture content below 5% g H₂O/g material. 500 g of the resulting dry material was charged into a 5 L glass reaction vessel equipped with an electric heating jacket, a nitrogen input line, an exhaust line directed to a scrubber with a water bubbler apparatus, and top and bottom thermocouples that are accurate to 0.5° C. The atmosphere of the charged reaction vessel was purged with nitrogen for 10 minutes, after which the nitrogen flow was reduced to the minimum required to drive nitrogen through the scrubber apparatus and maintained through the reaction. The temperature was increased gradually over 30 minutes to 350° C., maintained for 4.0 hours, and then decreased to room temperature over a 30-minute period. The powder recovered from the reactor vessel was ground gently into a coarse powder to yield 214 g of the carbon support material.

### Example B2a

### Chloromethylation of the Carbon Support from Example Bla

To a 100 mL three-neck round bottom flask (RBF) equipped with a stir bar, a condenser and nitrogen line is suspended the carbon support from Example B1a in anhydrous dichloroethane (DCE). The stirred suspension is cooled to 0° C. using an ice-water bath with continuous nitrogen flow. To the stirred suspension is added anhydrous zinc chloride. Chloromethyl methylether is then added dropwise into the reaction over a period of 15 minutes. The mixture is warmed to room temperature and stirred at 50° C. for 6 hours. The product is recovered by vacuum filtration and washed sequentially with water, acetone and dichloromethane. The washed solid is dried at 40° C. under vacuum. The extent of chloromethylation is determined by elemental analysis and by gravimetry.

### Example B2b

### Chloroacylation of the Carbon Support from Example B1a

The carbon support from Example B1a was suspended in anhydrous dichloroethane (DCE) in a 100 mL three-neck round bottom flask (RBF) equipped with a stir bar, a condenser, and a nitrogen input line The stirred suspension was cooled to 0° C. using an ice-water bath with continuous nitrogen flow. To the stirred suspension was added anhydrous aluminum chloride. Chloroacetyl chloride was then added dropwise into the reaction over a period of 15 minutes. The mixture is warmed to room temperature and stirred at 50° C. for 12 hours. The product was recovered by vacuum filtration and washed sequentially with water, acetone and dichloromethane. The washed solid was dried at 40° C. under vacuum to yield the final product. The extent of chloroacylation was determined to be 3.0 mmol chloride per gram of dry material by elemental analysis and by gravimetry.

### Example B2c

### Chloromethylation of Biochar

The carbon support from Example B1a is suspended in anhydrous dichloroethane (DCE) in a 100 mL three-neck round bottom flask (RBF) equipped with a stir bar, a condenser and nitrogen line. The stirred suspension is cooled to 0° C. using an ice-water bath with continuous nitrogen flow. To the stirred suspension is added anhydrous zinc chloride. Chloromethyl methylether is then added dropwise into the reaction over a period of 15 minutes. The mixture is warmed to room temperature and stirred at 50° C. for 6 hours. The product is recovered by vacuum filtration and washed sequentially with water, acetone and dichloromethane. The washed solid is dried at 40° C. under vacuum. The extent of chloromethylation is determined by elemental analysis and by gravimetry.

### Example B3a

### Quaternization of the Alkylated Carbon Support from Example B2a

To a 500 mL flask equipped with a magnetic stir bar and a condenser was charged acetone (100 ml), 1-methylimidazole (4.6 g, 56 mmol), 4-methylmorpholine (5.7 g, 56 mmol), and triphenylphosphine (14.7, 56 mmol). The resulting mixture was stirred at 50° C. for 10 minutes. In the stirred solution was suspended 40 g of the chloromethylated carbon support obtained from Example B2a. The resulting reaction mixture was heated under reflux conditions for 24 hours. After cooling, the reaction mixture was filtered using a fritted glass funnel under vacuum, washed sequentially with acetone and ethyl acetate, and dried overnight at 70° C. The extent of quaternization was determined by ion exchange titrimetry of Cl⁻ against AgNO₃.

### Example B3b

### Quaternization of the Alkylated Carbon Support from Example B2b

To a 500 mL flask equipped with a magnetic stir bar and a condenser was charged acetone (100 ml), 1-methylimidazole (4.6 g, 56 mmol), 4-methylmorpholine (5.7 g, 56 mmol), and triphenylphosphine (14.7, 56 mmol). The resulting mixture was stirred at 50° C. for 10 minutes. In the stirred solution was suspended 40 g of the chloroacylated carbon support obtained from Example B2b. The resulting reaction mixture was heated under reflux conditions for 24 hours. After cooling, the reaction mixture was filtered using a fritted glass funnel under vacuum, washed sequentially with acetone and ethyl acetate, and dried overnight at 70° C. The extent of quaternization was determined to be 1.7 mmol Cl⁻ per gram of dry solid by ion exchange titrimetry of Cl⁻ against AgNO₃.

### Example B4a

### Sulfonation of the Quaternized Carbon Support from Example B3a

To a 500 mL flask equipped with a magnetic stir bar and condenser is charged fuming sulfuric acid (20% free SO₃, 50 mL) and concentrated sulfuric acid (>95% w/w, ACS Reagent Grade, 50 mL). To the stirred acid is added 30 g of the quaternized carbon support obtained from Example B3a to form a dark black suspension. The mixture is stirred for 4 hours at 90° C. After cooling to room temperature, the reaction mixture is filtered using fritted glass funnel under vacuum, and then washed repeatedly with de-ionized water until the effluent is neutral, as determined by pH paper. The sulfonated support is air-dried to a final moisture content of 50% g H₂O/g wet polymer. The chemical functionalization of the support with sulfonic acid groups is determined by acid-base titration against sodium hydroxide.

### Example B4b

### Sulfonation of the Quaternized Carbon Support from Example B3b

To a 500 mL flask equipped with a magnetic stir bar and condenser was charged fuming sulfuric acid (20% free SO₃, 50 mL) and concentrated sulfuric acid (>95% w/w, ACS Reagent Grade, 50 mL). To the stirred acid was added 30 g of the quaternized carbon support from Example B3b to form a dark black suspension. The mixture was stirred for 4 hours at 90° C. After cooling to room temperature, the reaction mixture was filtered using fritted glass funnel under vacuum, and then washed repeatedly with de-ionized water until the effluent was neutral, as determined by pH paper. The sulfonated support was air-dried to a final moisture content of 56% g H₂O/g wet polymer. The chemical functionalization of the support resin with sulfonic acid groups was determined to be 3.65 mmol/g dry resin.

### Preparation of Solid Base Catalysts from Solid Acid Catalysts

### Example C1

### Preparation of Polymeric catalyst from Polymeric Catalyst of Example A3

A 1-inch diameter column equipped with a fritted glass plate and a 1 L solvent reservoir was charged with 250 mL of deionized water at room temperature. 20.0 g of the catalyst prepared according to the protocol set forth in Example A3 was added slowly to the column and allowed to settle. Gentle rotary mixing was performed during the settling to avoid the formation of any channels or air pockets in the packed bed formed by the settled resin. The excess water was eluted from the column by draining until the point that the column water level was 1 cm above the top of the packed resin bed. The column reservoir was then filled with 750 mL of 0.2 molar aqueous sodium hydroxide solution. The resin was washed with the sodium hydroxide solution by eluting the solution gradually from the column with a flow rate of approximately 1 mL per second. After elution with the 750 mL of sodium hydroxide solution, the resin was washed with 3 L of deionized water, until the effluent eluted with a pH below 8.0, as determined by pH paper. The resulting base catalyst was removed from the column and dried in air to a residual moisture content of below 50% g water per gram wet catalyst.

### Example C2

### Preparation of Base Catalysts with Monovalent Ions

This following experiment to prepare a base catalysts with movovalent ions from an acid catalyst is performed for each of the following acid catalysts prepared according to the procedure set forth in Examples A3, A4, A5, A7, A8, A10, A12, A14, A16, A18, A20, A22, A23, A25, A26, A28, A29, A30, A33, A34, A35, A36, A37, A38, A39, A42, A44, A46, A47, A50, A53, A55, A57, A59, A61, A63, A65, A67, A69, A71, B4a, and B4b.

A 1-inch diameter column equipped with a fritted glass plate and a 1 L solvent reservoir is charged with 250 mL of deionized water at room temperature. 20.0 g of the acid catalyst is added slowly to the column and allowed to settle. Gentle rotary mixing is performed during the settling to avoid the formation of any channels or air pockets in the packed bed formed by the settled resin. The excess water is eluted from the column by draining until the point that the column water level is one centimeter above the top of the packed resin bed. The column reservoir is then filled with 750 mL of 0.2 molar aqueous base solution. The base is selected from sodium hydroxide, potassium hydroxide, and ammonium hydroxide. The resin is washed with the base solution by eluting the solution gradually from the column with a flow rate of approximately 1 mL per second. After elution with the 750 mL of the base solution, the resin is washed with 3 L of deionized water, until the effluent elutes with a pH below 8.0, as determined by pH paper. The resulting base catalyst is removed from each column and dried in air to a residual moisture content of below 50% g water per gram wet catalyst.

### Example C3

### Preparation of Base Catalysts with Divalent Ions

This following experiment to prepare a base catalysts with divalent ions from an acid catalyst is performed for each of the following acid catalysts prepared according to the procedure set forth in Examples A3, A4, A5, A7, A8, A10, A12, A14, A16, A18, A20, A22, A23, A25, A26, A28, A29, A30, A33, A34, A35, A36, A37, A38, A39, A42, A44, A46, A47, A50, A53, A55, A57, A59, A61, A63, A65, A67, A69, A71, B4a, and B4b.

A 1-inch diameter column equipped with a fritted glass plate and a 1 L solvent reservoir is charged with 250 mL of deionized water at room temperature. 20.0 g of the acid catalyst is added slowly to the column and allowed to settle. Gentle rotary mixing is performed during the settling to avoid the formation of any channels or air pockets in the packed bed formed by the settled resin. The excess water is eluted from the column by draining until the point that the column water level is one centimeter above the top of the packed resin bed. A saturated base solution is prepared by dissolving 5 g of a base selected from calcium hydroxide, magnesium hydroxide, and lead hydroxide, in 750 mL of 0.1 molar aqueous sodium hydroxide solution. The resulting base solution is added to the column reservoir, and the resin is washed with the base solution by eluting the solution gradually from the column with a flow rate of approximately 1 mL per second. After elution with the 750 mL of the base solution, the resin is washed with 3 L of deionized water, until the effluent elutes with a pH below 8.0, as determined by pH paper. The resulting base catalyst is removed from the column and dried in air to a residual moisture content of below 50% g water per gram wet catalyst.

### Example C4

### Preparation of poly[sodium styrene-co-4-vinylbenzenesulfonate-co-3-methyl-1-(4-vinylbenzyl)-3H-imidazol-1-ium hydroxide-co-divinylbenzene]

A 1-inch diameter column equipped with a fritted glass plate and a 1 L solvent reservoir was charged with 250 mL of deionized water at room temperature. 24.1 g of poly[styrene-co-4-vinylbenzenesulfonic acid-co-3-methyl-1-(4-vinylbenzyl)-3H-imidazol-1-ium bisulfate-co-divinylbenzene], from Example A3, was added slowly to the column and allowed to settle. Gentle rotary mixing was performed during the settling to prevent the formation of any channels or air pockets in the packed bed formed by the settled resin. The excess water was eluted from the column by draining until the point that the column water level was one centimeter above the top of the packed resin bed. The column reservoir was then filled with 750 mL of 0.2 molar aqueous sodium hydroxide solution. The resin was washed with the base solution by eluting the solution gradually from the column with a flow rate of approximately 1 mL per second. After elution with the 750 mL of the base solution, the resin was washed with 3 L of deionized water. The final effluent eluted with a pH below 8.0, as determined by pH paper. The resulting base catalyst was removed from the column and dried in air for 24 hours. The residual moisture content of the resin was determined to be 32.56% g H₂O/g wet resin using a Mettler-Toledo MJ-33 moisture analyzing balance with a 0.6 g sample size.

### Digestion of Lignin Compositions Using Solid Base Catalysts

### Example D1

### Digestion of Sugarcane Bagasse Using Catalyst Described in Example A3

Sugarcane bagasse (50% g H₂O/g wet bagasse, with a dry-matter composition of: 39.0% g glucan/g dry biomass, 17.3% g xylan/g dry biomass, 5.0% g arabinan/g dry biomass, 1.1% g galactan/g dry biomass, 5.5% g acetate/g dry biomass, 5.0% g soluble extractives/g dry biomass, 24.1% g lignin/g dry biomass, and 3.1% g ash/g dry biomass) was cut such that the maximum particle size was no greater than 1 cm. The composition of the lignocellulosic biomass was determined using a method based on the procedures known in the art. See R. Ruiz and T. Ehrman, “Determination of Carbohydrates in Biomass by High Performance Liquid Chromatography,” NREL Laboratory Analytical Procedure LAP-002 (1996); D. Tempelton and T. Ehrman, “Determination of Acid-Insoluble Lignin in Biomass,” NREL Laboratory Analytical Procedure LAP-003 (1995); T. Erhman, “Determination of Acid-Soluble Lignin in Biomass,” NREL Laboratory Analytical Procedure LAP-004 (1996); and T. Ehrman, “Standard Method for Ash in Biomass,” NREL Laboratory Analytical Procedure LAP-005 (1994).

To a 15 mL cylindrical glass reaction vial was added: 0.50 g of the cane bagasse sample, 0.30 g of Catalyst as prepared in Example A3 (initial moisture content: 12% g H₂O/g dispensed catalyst), and 800 μL of deionized H₂O. The reactants were mixed thoroughly with a glass stir rod to distribute the catalyst particles evenly throughout the biomass. The resulting mixture was gently compacted to yield a solid reactant cake. The glass reactor was incubated at 120° C. for four hours.

### Example D2

### Separation of Catalyst/Product Mixture from the Hydrolysis of Sugarcane Bagasse

The cylindrical glass reactor from Example C1 was cooled to room temperature and unsealed. 10.0 mL of distilled H₂O was added to the vial reactor and the resulting mixture of liquids and solids was agitated for 2 minutes by magnetic stirring. Following agitation, the solids were allowed to sediment for 30 seconds to produce the layered mixture. The solid catalyst formed a layer at the bottom of the vial reactor, and the residual biomass formed a solid layer above the solid catalyst. The aqueous phase was recovered from the solid-liquid mixture by vacuum filtration through a 20 micron frit. 8.4 mL of liquid were recovered. The presence of solubilized phenolic species was confirmed by measuring the absorbance of the eluate at 210 nm UV-VIS spectroscopy.

### Example D3

### Steam Treatment of Lignin

To a 20 mL serum vial was added 1.0447 g of lignin (Sigma-Aldrich, Catalog Number 370959, Moisture Content 4.92% g H2O/g lignin) and 2.75 mL of de-ionized water. The vial was sealed with a PTFE septum held by an aluminum crimp top and then incubated at 130° C. for 2.5 hours in a laboratory oven. The vial was then removed from the oven, allowed to cool to room temperature, and opened by removing the aluminum seal and septum. A clean ceramic filter crucible was dried to constant mass at 105° C. and weighed to determine its empty mass, accurate to 0.1 mg. The contents of the vial were transferred to the empty filter crucible using excess water to transfer the solids quantitatively from the vial to the crucible. The solid species were vacuum-filtered and washed three times with 25 mL each of de-ionized water and 25 mL of acetone. The crucible containing the residual solids was dried to constant mass at 105° C. and weighed to determine the mass of residual lignin. By gravimetric analysis, the fraction of lignin that was solubilized was determined to be 10.6% g/g.

### Example D4

### Solubilization of Lignin Using the Catalyst from Example C4

To a 20 mL serum vial was added 1.0335 g of lignin (Sigma-Aldrich, Catalog Number 370959, Moisture Content 4.92% g H2O/g lignin), 2.5466 g of the catalyst from Example C4 (moisture content of 32.56% g H2O/g catalyst) and 2.00 mL of de-ionized water. The vial was sealed with a PTFE septum held by an aluminum crimp top and then incubated at 130° C. for 2.5 hours in a laboratory oven. The vial was then removed from the oven, allowed to cool to room temperature, and opened by removing the aluminum seal and septum. A clean ceramic filter crucible was dried to constant mass at 105° C. and weighed to determine its empty mass, accurate to 0.1 mg. The contents of the vial were transferred to the empty filter crucible using excess water to transfer the solids quantitatively from the vial to the crucible. The solid species were vacuum-filtered and washed three times with 25 mL each of de-ionized water and 25 mL of acetone. The crucible containing the residual solids was dried to constant mass at 105° C. and weighed to determine the mass of residual lignin. By gravimetric analysis, the fraction of lignin that was solubilized was determined to be 79.4% g/g.

1. A catalyst comprising basic monomers and ionic monomers connected to form a polymeric backbone,
wherein each basic monomer independently comprises at least one Bronsted-Lowry base, wherein each Bronsted-Lowry base independently comprises at least one nitrogen-containing cationic group, at least one phosphorous-containing cationic group, at least one sulfur-containing cationic group, or any combinations thereof.

2. The catalyst of claim 1, wherein each Bronsted-Lowry base is selected from the group consisting of pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.

3. The catalyst of claim 1, wherein each ionic monomer comprises at least one anionic group, wherein each anionic group is independently selected from the group consisting of sulfonate, phosphonate, acetate, isophthalate, and boronate.

4. The catalyst of claim 1, wherein the polymeric backbone is selected from the group consisting of polyethylene, polypropylene, polyvinyl alcohol, polystyrene, polyurethane, polyvinyl chloride, polyphenol-aldehyde, polytetrafluoroethylene, polybutylene terephthalate, polycaprolactam, poly(acrylonitrile butadiene styrene), polyalkyleneammonium, polyalkylenediammonium, polyalkylenepyrrolium, polyalkyleneimidazolium, polyalkylenepyrazolium, polyalkyleneoxazolium, polyalkylenethiazolium, polyalkylenepyridinium, polyalkylenepyrimidinium, polyalkylenepyrazinium, polyalkylenepyradizimium, polyalkylenethiazinium, polyalkylenemorpholinium, polyalkylenepiperidinium, polyalkylenepiperizinium, polyalkylenepyrollizinium, polyalkylenetriphenylphosphonium, polyalkylenetrimethylphosphonium, polyalkylenetriethylphosphonium, polyalkylenetripropylphosphonium, polyalkylenetributylphosphonium, polyalkylenetrichlorophosphonium, polyalkylenetrifluorophosphonium, and polyalkylenediazolium.

5. The catalyst of claim 1, wherein one or more of the basic monomers each further comprise a linker connecting the Bronsted-Lowry base to the polymeric backbone; and/or wherein one or more of the ionic monomers each further comprise a linker connecting the anionic monomers to the polymeric backbone.

6. A catalyst comprising a solid support, basic moieties attached to the solid support, and ionic moieties attached to the solid support,
wherein the solid support comprises a material, wherein the material is selected from the group consisting of carbon, silica, silica gel, alumina, magnesia, titania, zirconia, clays, magnesium silicate, silicon carbide, zeolites, ceramics, and any combinations thereof,
wherein each basic moiety independently comprises at least one Bronsted-Lowry base, wherein each Bronsted-Lowry base independently comprises at least one nitrogen-containing cationic group, at least one phosphorous-containing cationic group, at least one sulfur-containing cationic group, or any combinations thereof

7. The catalyst of claim 6, wherein the material is carbon.

8. The catalyst of claim 7, wherein the carbon is selected from the group consisting of biochar, amorphous carbon, and activated carbon.

9. The catalyst of claim 6, wherein each Bronsted-Lowry base is selected from the group consisting of pyrrolium hydroxide, imidazolium hydroxide, pyrazolium hydroxide, oxazolium hydroxide, thiazolium hydroxide, pyridinium hydroxide, pyrimidinium hydroxide, pyrazinium hydroxide, pyradizimium hydroxide, thiazinium hydroxide, morpholinium hydroxide, piperidinium hydroxide, piperizinium hydroxide, pyrollizinium hydroxide, phosphonium hydroxide, trimethyl phosphonium hydroxide, triethyl phosphonium hydroxide, tripropyl phosphonium hydroxide, tributyl phosphonium hydroxide, trichloro phosphonium hydroxide, triphenyl phosphonium hydroxide, trifluoro phosphonium hydroxide, sulfonium hydroxide, methylsulfonium hydroxide, dimethylsulfonium hydroxide, trimethylsulfonium hydroxide, tetramethylsulfonium hydroxide, ethylsulfonium hydroxide, diethylsulfonium hydroxide, triethylsulfonium hydroxide, tetraethylsulfonium hydroxide, propylsulfonium hydroxide, dipropylsulfonium hydroxide, tripropylsulfonium hydroxide, tetrapropylsulfonium hydroxide, butylsulfonium hydroxide, dibutylsulfonium hydroxide, tributylsulfonium hydroxide, tetrabutylsulfonium hydroxide, phenylsulfonium hydroxide, diphenylsulfonium hydroxide, triphenylsulfonium hydroxide, and tetraphenylsulonium hydroxide.

10. The catalyst of claim 6, wherein each ionic moiety is independently selected from the group consisting of a sulfonate salt, a phosphonate salt, an acetate salt, an isophthalate salt, and a boronate salt.

11. The catalyst of claim 1, wherein one or more of the basic moieties each further comprise a linker attaching the Bronsted-Lowry base to the solid support; and/or wherein one or more of the ionic moieties each further comprise a linker attaching the anionic moieties to the solid support.

12. A composition comprising:
lignin; and
a catalyst of claim 1.

13. A partially-depolymerized lignin composition comprising:
a catalyst of claim 1;
one or more lignin digestion products; and
residual lignin.

14. The composition of claim 13, wherein the one or more lignin digestion products are selected from the group consisting of monolignols, phenylpropenes, monolignolglucosides, and any combinations thereof

15. The composition of claim 13, wherein the one or more lignin digestion products are selected from the group consisting of p-coumaryl alcohol, coumarilin, coniferyl alchol, coniferin, sinapyl alcohol, sinaplin, eugenol, chavicol, safrole, estragol, and any combinations thereof

16. A method for at least partially depolymerizing a lignin composition,
comprising:
a) providing a lignin composition;
b) contacting the lignin composition with a catalyst of claim 1 and one or more solvents to form a reaction mixture;
c) degrading the lignin composition in the reaction mixture to produce a liquid phase and a solid phase, wherein the liquid phase comprises one or more lignin digestion products, and the solid phase comprises residual lignin;
d) isolating at least a portion of the liquid phase from the solid phase; and
e) recovering the one or more lignin digestion products from the isolated liquid phase.

17. The method of claim 16, wherein the one or more lignin digestion products are selected from the group consisting of monolignols, phenylpropenes, monolignolglucosides, and any combinations thereof

18. The method of claim 16, wherein the one or more lignin digestion products are selected from the group consisting of p-coumaryl alcohol, coumarilin, coniferyl alchol, coniferin, sinapyl alcohol, sinaplin, eugenol, chavicol, safrole, estragol, and any combinations thereof.

19. (canceled)
